SEQUENCE LISTING (32 sequences)

SEQ ID NO: 1 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial sequence [sequence omitted]

SEQ ID NO: 3 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 4 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 5 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 6 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 14 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 499 aa, Artificial sequence
  sequence is synthesized
MDFQVQIFSFLLISASVIMSRGQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKDGGGSGGGGSGGGGSSQAYLQQSGAESVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 106 aa, Artificial sequence
  sequence is synthesized
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIK

SEQ ID NO: 18 - protein, 121 aa, Artificial sequence
  sequence is synthesized
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSS

SEQ ID NO: 19 - protein, 213 aa, Artificial sequence
  sequence is synthesized
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 121 aa, Artificial sequence; identical to SEQ ID NO: 18
  sequence is synthesized

SEQ ID NO: 21 - protein, 765 aa, Artificial sequence [sequence omitted]

SEQ ID NO: 22 - protein, 115 aa, Artificial sequence
  sequence is synthesized
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 23 - protein, 119 aa, Artificial sequence
  sequence is synthesized
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 24 - protein, 106 aa, Artificial sequence [sequence omitted]

SEQ ID NO: 25 - protein, 121 aa, Artificial sequence
  sequence is synthesized
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS

SEQ ID NO: 26 - protein, 5 aa, Artificial sequence
  sequence is synthesized
QKRAA

SEQ ID NO: 27 - protein, 5 aa, Artificial sequence
  sequence is synthesized
QRRAA

SEQ ID NO: 28 - protein, 5 aa, Artificial sequence
  sequence is synthesized
RRRAA

SEQ ID NO: 29 - DNA, 20 bp, Artificial sequence
  sequence is synthesized
gttgcgcaggctagtcttga

SEQ ID NO: 30 - DNA, 40 bp, Artificial sequence
  sequence is synthesized
gctgctccggttcatagattggatagcaactgctccaagg

SEQ ID NO: 31 - DNA, 20 bp, Artificial sequence
  sequence is synthesized
gctgctccggttcatagatt

SEQ ID NO: 32 - DNA, 17 bp, Artificial sequence
  sequence is synthesized
aaatgattcaggtgtcc

(19) United States
(12) Patent Application Publication
(10) Pub. No.: US 2009/0204489 A1
(43) Pub. Date: Aug. 13, 2009

(54) BIOLOGICAL MARKERS PREDICTIVE OF RHEUMATOID ARTHRITIS RESPONSE TO B-CELL ANTAGONISTS

(75) Inventors: TIMOTHY BEHRENS, BURLINGAME, CA (US); THARAKNATH RAO, BASEL (CH)

Correspondence Address: GENENTECH, INC., 1 DNA WAY, SOUTH SAN FRANCISCO, CA, 94080, US

(73) Assignee: GENENTECH, INC., SOUTH SAN FRANCISCO, CA (US)

(21) Appl. No.: 12/060,572

(22) Filed: Apr. 1, 2008

Related U.S. Application Data
(60) Provisional application No. 60/909,921, filed on Apr. 3, 2007.
(60) Provisional application No. 60/909,693, filed on Apr. 2, 2007.

(51) Int. Cl. G06Q 30/00; A61K 39/395; A61K 38/16; A61K 39/44; A61K 31/519; A61K 31/56; C12Q 1/68; G06N 5/00

(52) U.S. Cl. 705/14; 424/130.1; 514/12; 424/133.1; 424/178.1; 514/262.1; 514/171; 435/6; 706/45

(57) ABSTRACT

Methods and assays examining expression of one or more biomarkers in a sample are provided for predicting or indicating the effectiveness of treatment of a rheumatoid arthritis (RA) patient with a B-cell antagonist. Methods are provided for identifying patients whose RA is likely to be responsive to anti-RA therapy using a B-cell-antagonist. Methods for treating such patients with B-cell antagonists that incorporate the above methodology are also provided. Further provided are kits and articles of manufacture useful for such methods.

### RELATED APPLICATIONS

This application claims priority to U.S. Provisional Application Ser. No. 60/909,693 filed on Apr. 2, 2007 and U.S. Provisional Application Ser. No. 60/909,921 filed on Apr. 3, 2007, both of which are incorporated herein by reference in their entirety.

### FIELD OF THE INVENTION

The present invention concerns methods for diagnosing and treating rheumatoid arthritis (RA) patients. In particular, the present invention is directed to methods for determining which patients will most benefit from treatment with B-cell antagonist therapies directed against B-cell surface markers or B-cell specific proliferation or survival factors, such as an antibody or immunoadhesin.

### BACKGROUND OF THE INVENTION

### Joint Destruction and Damage

Autoimmune diseases remain clinically important diseases in humans. As the name implies, autoimmune diseases act through the body's own immune system. While the pathological mechanisms differ among individual types of autoimmune diseases, one general mechanism involves the generation of antibodies (referred to herein as self-reactive antibodies or autoantibodies) directed against specific endogenous proteins. Physicians and scientists have identified more than 70 clinically distinct autoimmune diseases, including RA, multiple sclerosis (MS), vasculitis, immune-mediated diabetes, and lupus such as systemic lupus erythematosus (SLE). While many autoimmune diseases are rare—affecting fewer than 200,000 individuals—collectively, these diseases afflict millions of Americans, an estimated five percent of the population, with women disproportionately affected by most diseases. The chronic nature of these diseases leads to an immense social and financial burden.

Inflammatory arthritis is a prominent clinical manifestation in diverse autoimmune disorders including RA, psoriatic arthritis (PsA), SLE, Sjögren's syndrome, and polymyositis. Most of these patients develop joint deformities on physical examination but typically only RA and PsA patients manifest bone erosions on imaging studies.

RA is a chronic inflammatory disease that affects approximately 0.5 to 1% of the adult population in northern Europe and North America, and a slightly lower proportion in other parts of the world. Alamanos and Drosos, Autoimmun. Rev., 4: 130-136 (2005). It is a systemic inflammatory disease characterized by chronic inflammation in the synovial membrane of affected joints, which ultimately leads to loss of daily function due to chronic pain and fatigue. The majority of patients also experience progressive deterioration of cartilage and bone in the affected joints, which may eventually lead to permanent disability. The long-term prognosis of RA is poor, with approximately 50% of patients experiencing significant functional disability within 10 years from the time of diagnosis. Keystone, Rheumatology, 44 (Suppl. 2): ii8-ii12 (2005). Life expectancy is reduced by an average of 3-10 years. Alamanos and Drosos, supra. Patients with a high titer of rheumatoid factor (RF) (approximately 80% of patients) have more aggressive disease (Bukhari et al., Arthritis Rheum., 46: 906-912 (2002)), with a worse long-term outcome and increased mortality over those who are RF negative. Heliovaara et al., Ann. Rheum. Dis., 54: 811-814 (1995)).

The pathogenesis of chronic inflammatory bone diseases, such as RA, is not fully elucidated. Such diseases are accompanied by bone loss around affected joints due to increased osteoclastic resorption. This process is mediated largely by increased local production of pro-inflammatory cytokines. Teitelbaum, Science, 289:1504-1508 (2000); Goldring and Gravallese, Arthritis Res., 2(1):33-37 (2000). These cytokines can act directly on cells in the osteoclast lineage or indirectly by affecting the production of the essential osteoclast differentiation factor, receptor activator of NFκB ligand (RANKL), and/or its soluble decoy receptor, osteoprotegerin (OPG), by osteoblast/stromal cells. Hossbauer et al., J. Bone Min. Res., 15(1):2-12 (2000). Tumor necrosis factor-alpha (TNF-α) is a major mediator of inflammation. Its importance in the pathogenesis of various forms of bone loss is supported by several lines of experimental and clinical evidence. Feldmann et al., Cell, 85(3):307-310 (1996). However, TNF-α is not essential for osteoclastogenesis (Douni et al., J. Inflamm., 47:27-38 (1996)), erosive arthritis (Campbell et al., J. Clin. Invest., 107(12):1519-1527 (2001)), or osteolysis (Childs et al., J. Bon. Min. Res., 16:338-347 (2001)), as these can occur in the absence of TNF-α.

In RA specifically, an immune response is thought to be initiated/perpetuated by one or several antigens presenting in the synovial compartment, producing an influx of acute inflammatory cells and lymphocytes into the joint. Successive waves of inflammation lead to the formation of an invasive and erosive tissue called pannus. This contains proliferating fibroblast-like synoviocytes and macrophages that produce proinflammatory cytokines such as TNF-α and interleukin-1 (IL-1). Local release of proteolytic enzymes, various inflammatory mediators, and osteoclast activation contributes to much of the tissue damage. There is loss of articular cartilage and the formation of bony erosions. Surrounding tendons and bursa may become affected by the inflammatory process. Ultimately, the integrity of the joint structure is compromised, producing disability.

The precise contributions of B cells to the immunopathogenesis of RA are not completely characterized. However, there are several possible mechanisms by which B cells may participate in the disease process. Silverman and Carson, Arthritis Res. Ther., 5 Suppl. 4: S1-6 (2003).

Historically, B cells were thought to contribute to the disease process in RA predominantly by serving as the precursors of autoantibody-producing cells. A number of autoantibody specificities have been identified including antibodies to Type II collagen, and proteoglycans, as well as RFs. The generation of large quantities of antibody leads to immune complex formation and the activation of the complement cascade. This in turn amplifies the immune response and may culminate in local cell lysis. Increased RF synthesis and complement consumption has been correlated with disease activity. The presence of RF itself is associated with a more severe form of RA and the presence of extra-articular features.

Evidence exists (Janeway and Katz, J. Immunol., 138:1051 (1998); Rivera et al., Int. Immunol., 13: 1583-1593 (2001)) showing that B cells are highly efficient antigen-presenting cells (APC). RF-positive B cells may be particularly potent APCs, since their surface immunoglobulin would readily allow capture of any immune complexes regardless of the antigens present within them. Many antigens may thus be processed for presentation to T cells. In addition, it has been recently suggested that this may also allow RF-positive B cells to self-perpetuate. Edwards et al., Immunology, 97: 188-196 (1999).

For activation of T cells, two signals need to be delivered to the cell; one via the T-cell receptor (TCR), which recognizes the processed peptide in the presence of major histocompatibility complex (MHC) antigen, and a second, via co-stimulatory molecules. When activated, B cells express co-stimulatory molecules on their surface and can thus provide the second signal for T-cell activation and the generation of effector cells.

B cells may promote their own function as well as that of other cells by producing cytokines. Harris et al., Nat. Immunol., 1: 475-482 (2000). TNF-α, IL-1, lymphotoxin-α, IL-6, and IL-10 are amongst some of the cytokines that B cells may produce in the RA synovium.

Although T-cell activation is considered to be a key component in the pathogenesis of RA, recent work using human synovium explants in severe combined immunodeficiency disorders (SCID) mice has demonstrated that T-cell activation and retention within the joint is critically dependent on the presence of B cells. Takemura et al., J. Immunol., 167: 4710-4718 (2001). The precise role of B cells in this is unclear, since other APCs did not appear to have the same effect on T cells.

Structural damage to joints is an important consequence of chronic synovial inflammation. Between 60% and 95% of patients with RA develop at least one radiographic erosion within 3-8 years of disease onset. Paulus et al., J. Rheumatol., 23: 801-805 (1996); Hulsmans et al., Arthritis Rheum., 43: 1927-1940 (2000). In early RA, the correlation between radiographic damage scores and functional capacity is weak, but after 8 years of disease, correlation coefficients can reach as high as 0.68. Scott et al., Rheumatology, 39:122-132 (2000). In 1,007 patients younger than age 60 years who had RA for at least four years, Wolfe et al. (Arthritis Rheum, 43 Suppl. 9:S403 (2000)) found a significant association among the rate of progression of the Larsen radiographic damage score (Larsen et al., Acta Radiol. Diagn. 18:481-491 (1977)), increasing Social Security disability status, and decreasing family income.

Prevention or retardation of radiographic damage is one of the goals of RA treatment. Edmonds et al., Arthritis Rheum., 36:336-340 (1993). Controlled clinical trials of 6 or 12 months' duration have documented that the progression of radiographic damage scores was more rapid in the placebo group than in groups that received methotrexate (MTX) (Sharp et al., Arthritis Rheum., 43: 495-505 (2000)), leflunomide (Sharp et al., supra), sulfasalazine (SSZ) (Sharp et al., supra), prednisolone (Kirwan et al., N. Engl. J. Med., 333:142-146 (1995); Wassenburg et al., Arthritis Rheum, 42: Suppl 9:S243 (1999)), interleukin-1 receptor antagonist (Bresnihan et al., Arthritis Rheum, 41: 2196-2204 (1998)), or an infliximab/MTX combination. Lipsky et al., N. Eng. J. Med., 343: 1594-1604 (2000). Clinical trials have also documented that radiographic progression following treatment with etanercept was less rapid than that following treatment with MTX. Bathon et al., N. Engl. J. Med., 343:1586-1593 (2000). Other studies have evaluated radiographic progression in patients treated with corticosteroids (Joint Committee of the Medical Research Council and Nuffield Foundation, Ann Rheum. Dis., 19:331-337 (1960); Van Everdingen et al., Ann. Intern. Med., 136:1-12 (2002)), cyclosporin A (Pasero et al., J. Rheumatol., 24:2113-2118 (1997); Forre, Arthritis Rheum., 37:1506-1512 (1994)), MTX versus azathioprine (Jeurissen et al., Ann. Intern. Med., 114:999-1004 (1991)), MTX versus auranofin (Weinblatt et al., Arthritis Rheum., 36:613-619 (1993)), MTX (meta-analysis) (Alarcon et al., J. Rheumatol., 19:1868-1873 (1992)), hydroxychloroquine (HCQ) versus SSZ (Van der Heijde et al., Lancet, 1: 1036-1038), SSZ (Hannonen et al., Arthritis Rheum., 36:1501-1509 (1993)), the COBRA (Combinatietherapei Bij Reumatoide Artritis) combination of prednisolone, MTX, and SSZ (Boers et al., Lancet, 350:309-318 (1997); Landewe et al., Arthritis Rheum., 46: 347-356 (2002)), combinations of MTX, SSZ, and HCQ (O'Dell et al., N. Engl. J. Med., 334:1287-1291 (1996); Mottonen et al., Lancet, 353:1568-1573 (1999)), the combination of cyclophosphamide, azathioprine, and HCQ (Csuka et al., JAMA, 255:2115-2119 (1986)), and the combination of adalimumab with MTX. Keystone et al., Arthritis Rheum., 46 Suppl. 9:S205 (2002).

The FDA has now approved labeling claims that certain medications, e.g., leflunomide, etanercept, and infliximab, slow the progression of radiographic joint damage. These claims are based on the statistically significant differences in progression rates observed between randomly assigned treatment groups and control groups. However, the progression rates in individuals within the treatment and control groups overlap to a considerable extent. Therefore, despite significant differences between treatment groups, these data cannot be used to estimate the probability that a patient who is starting a treatment will have a favorable outcome with respect to progression of radiographic damage. Various methods have been suggested to categorize paired radiographs from individual patients as not progressive, e.g., damage scores of 0 at both time points, no increase in damage scores, no new joints with erosions, and a change in score not exceeding the smallest detectable difference (i.e., 95% confidence interval for the difference between repeated readings of the same radiograph). Lassere et al., J. Rheumatol., 26: 731-739 (1999).

Determining whether there has been increased structural damage in an individual patient during the interval between paired radiographs obtained at the beginning and end of a 6- or 12-month clinical trial has been difficult, for several reasons. The rate of radiographic damage is not uniform within a population of RA patients; a few patients may have rapidly progressing damage, but many may have little or no progression, especially if the tie interval is relatively short. The methods for scoring radiographic damage, e.g., Sharp (Sharp et al., Arthritis Rheum., 14: 706-720 (1971); Sharp et al., Arthritis Rheum., 28: 1326-1335 (1985)), Larsen (Larsen et al., Acta Radiol. Diagn., 18: 481-491 (1977)), and modifications of these methods (Van der Heijde, J. Rheumatol., 27: 261-263 (2000)), depend on the judgment and the interpretation of the reader as to what is real. Factors to determine are whether an apparent interruption of the subchondral cortical plate is real, or whether a decrease in the distance between the cortices on opposite sides of a joint is real, or is due to a slight change in the position of the joint relative to the film and the radiographic beam, to a change in radiographic exposure, or to some other technical factor.

Therefore, the recorded score is an approximation of the true damage, and for many subjects, the smallest detectable difference between repeat scores of the same radiographs is larger than the actual change that has occurred during the interval between the baseline and final radiographs. If the reader is blinded to the temporal sequence of the films, these unavoidable scoring errors may be in either direction, leading to apparent “healing” when the score decreases or to apparent rapid progression when reading error increases the difference between films. When the study involves a sufficiently large population of patients who have been randomly assigned to receive an effective treatment as compared with placebo, the positive and negative reading errors offset each other, and small but real differences between treatment groups can be detected.

The imprecision of the clinical measures that are used to quantitate RA disease activity has caused a similar problem. Statistically significant differences between certain outcome measures from clinical trials were not useful for estimating the probability of improvement for an individual who was starting the treatment. Paulus et al., Arthritis Rheum., 33:477-484 (1990). Attribution of individual improvement became practical with the creation of the American College of Rheumatology (ACR) 20% composite criteria for improvement (ACR20), which designated a patient as improved if there was 20% improvement in the tender and swollen joint counts and 20% improvement in at least three of five additional measures (pain, physical function, patient global health assessment, physician global health assessment, and acute-phase reactant levels). Felson et al., Arthritis Rheum., 38:727-735 (1995). All of these measures have large values for the smallest detectable difference, but by requiring simultaneous improvement in five of the seven aspects of the same process (disease activity), the randomness of the seven measurement errors is constrained, and it is easier to attribute real improvement to the individual.

In RA, joint damage is a prominent feature. Radiologic parameters of joint destruction are seen as a key outcome measure in descriptions of disease outcome. In the recent OMERACT (Outcome Measures in Rheumatology Clinical Trials) consensus meeting, radiology was chosen as part of the core set of outcome measures for longitudinal observational studies. Wolfe et al., Arthritis Rheum., 41 Supp 9: S204 (1998) abstract. Radiology is also part of the WHO/ILAR (World Health Organization/International League of Associations for Rheumatology) required core set of measures for long-term clinical trials. Tugwell and Boers, J. Rheumatol., 20:528-530 (1993).

Available data on the outcome of radiologic damage in RA have been obtained in both short-term and long-term studies. In short-term studies of RA patients with recent-onset disease, radiographs obtained every six months showed that after an initial rapid progression, there was diminution of the progression rate of radiologic damage in the hands and feet after two to three years. Van der Heijde et al., Arthritis Rheum., 35: 26-34 (1992); Fex et al., Br. J. Rheumatol., 35: 1106-1055 (1996). In long-term studies with radiographs taken less frequently, a constant rate of progression was found, with relentless deterioration of damage up to 25 years of disease duration. Wolfe and Sharp, Arthritis Rheum., 41:1571-1582 (1998); Graudal et al., Arthritis Rheum., 41:1470-1480 (1998); Plant et al., J. Rheumatol., 25:417-426 (1998); Kaarela and Kautiainen, J. Rheumatol., 24:1285-1287 (1997). Whether these differences in radiographic progression pattern are due to differences in the scoring techniques is not clear.

The scoring systems used differ in the number of joints being scored, the presence of independent scores for erosions (ERO) and joint space narrowing (JSN), the maximum score per joint, and the weighing of a radiologic abnormality. As yet, there is no consensus on the scoring method of preference. During the first three years of follow-up in a cohort study of patients with early arthritis, JSN and ERO were found to differ in their contribution to the measured progression in radiologic damage of the hands and feet. Van der Heijde et al., Arthritis Rheum., 35:26-34 (1992). Furthermore, methods that independently score ERO and JSN, such as the Sharp and Kellgren scores, were found to be more sensitive to change in early RA than methods using an overall measure, such as the Larsen score. Plant et al., J. Rheumatol., 21:1808-1813 (1994); Cuchacovich et al., Arthritis Rheum., 35:736-739 (1992). The Sharp score is a very labor-intensive method. Van der Heijde, Baillieres Clin. Rheumatol., 10:435-533 (1996). In late or destructive RA, the Sharp and the Larsen methods were found to provide similar information. However, the sensitivity to change of the various scoring methods late in the disease has not yet been investigated, and it can be argued that the scoring methods that independently measure ERO and JSN provide useful information. Pincus et al., J. Rheumatol., 24:2106-2122 (1997). See also Drossaers-Bakker et al., Arthritis Rheum., 43:1465-1472 (2000), which compared the three radiologic scoring systems for the long-term assessment of RA.

Paulus et al., Arthritis Rheum., 50: 1083-1096 (2004) categorized radiographic joint damage as progressive or non-progressive in individuals with RA participating in clinical trials, and concluded that RA joint damage in an observational cohort can be classified as progressive or non-progressive with the use of a composite definition that includes a number of imprecise and related, but distinct, measures of structural joint damage. It appears that in day-to-day clinical management of an RA patient, an interval change between a pair of radiographs of at least five Sharp radiographic damage score units should be present before one considers the structural change to be real and uses it as the basis for a treatment decision.

Over the past ten years there have been major advances in the treatment of RA. Combination use of existing disease-modifying anti-rheumatic drugs (DMARDs), together with new biologic agents, have provided higher levels of efficacy in a larger proportion of patients, while the early diagnosis and treatment of the disease has also improved outcomes.

Etanercept is a fully human fusion protein that inhibits TNF and the subsequent inflammatory cytokine cascade. Etanercept has been shown to be safe and effective in rapidly reducing disease activity in adults with RA and in sustaining that improvement. Bathon et al., N. Eng. J. Med., 343:1586-1593 (2000); Moreland et al., N. Engl. J. Med., 337:141-147 (1997); Moreland et al., Ann. Intern. Med., 130:478-486 (1999); Weinblatt et al., N. Engl. J. Med., 340:253-259 (1999); Moreland et al., J. Rheum., 28:1238-1244 (2001). It is equally effective in children with polyarticular juvenile RA. Lovell et al., N. Engl. J. Med., 342:763-769 (2000). Etanercept is approved for use as monotherapy, as well as in combination therapy with MTX, for the treatment of RA. US 2007/0071747 discloses use of a TNF-α inhibitor for treatment of erosive polyarthritis.

Loss of function and radiographic change occur early in the course of the disease. These changes can be delayed or prevented with the use of certain DMARDs. Although several DMARDs are initially clinically effective and well tolerated, many of these drugs become less effective or exhibit increased toxicity over time. Based on its efficacy and tolerability, MTX has become the standard therapy by which other treatments are measured. Bathon et al., N. Eng. J. Med., 343:1586-1593 (2000); Albert et al., J. Rheumatol., 27:644-652 (2000).

Recent studies have examined radiographic progression in patients with late-stage RA who have taken leflunomide, MTX, or placebo (Strand et al., Arch. Intern. Med., 159:2542-2550 (1999)) as well as patients who have taken infliximab plus MTX or placebo plus MTX following a partial response to MTX. Lipsky et al., N. Engl. J. Med., 343:1594-1602 (2000); Maini et al., Lancet, 354:1932-1939 (1999). In the first year of the ENBREL™ ERA (early RA) trial, etanercept was shown to be significantly more effective than MTX in improving signs and symptoms of disease and in inhibiting radiographic progression. Bathon et al., N. Eng. J. Med., 343:1586-1593 (2000). Genovese et al., Arthritis Rheum. 46:1443-1450 (2002) reports results from the second year of the study, concluding that etanercept as monotherapy was safe and superior to MTX in reducing disease activity, arresting structural damage, and decreasing disability over two years in patients with early aggressive RA. Also studied was the safety and clinical activity of ocrelizumab (a humanized antibody targeting C D20+B cells) in combination with MTX in moderate-to-severe RA patients (Ph I/II ACTION study). Genovese et al., Arthritis Rheum., 54(9):S66-S67 (September 2006).

Further, reduction in radiographic progression in the hands and feet was observed in patients with early RA after receiving infliximab in combination with MTX. Van der Heijde et al., Annals Rheumatic Diseases, 64:417 (2005). Patients with early RA achieved a clinically meaningful and sustained improvement in physical function after treatment with infliximab. Smolen et al., Annals Rheumatic Diseases, 64:418-419 (2005).

The effect of infliximab therapy on bone mineral density in patients with ankylosing spondylitis (AS) resulting from a randomized, placebo-controlled trial named ASSERT) is reported by Van der Heijde et al., Annals Rheumatic Diseases, 64:319 (2005). The ASSERT trial showed that infliximab improved fatigue and pain in patients with AS. Van der Heijde et al., Annals Rheumatic Diseases, 64:318-319 (2005). The efficacy and safety of infliximab in AS patients treated according to ASSERT are described by van der Heijde et al., Arthritis Rheum., 5:582-591 (2005). The authors conclude that infliximab was well tolerated and effective in a large cohort of patients with AS during a 24-week study period. In addition, the effect of infliximab therapy on spinal inflammation was assessed by magnetic resonance imaging in a randomized, placebo-controlled trial of 279 patients with AS. Van der Heijde et al., Annals Rheumatic Diseases, 64:317 (2005). The manner in which the treatment effect on spinal radiographic progression in patients with AS should be measured is addressed by van der Heijde et al., Arthritis Rheum. 52:1979-1985 (2005).

The results of radiographic analyses of the infliximab multinational PsA controlled trial (IMPACT) after one year are reported by Antoni et al., Annals Rheumatic Diseases 64:107 (2005). Evidence of radiographic benefit of treatment with infliximab plus MTX in RA patients who had no clinical improvement, with a detailed subanalysis of data from the anti-TNF trial in RA with concomitant therapy study, is reported by Smolen et al., Arthritis Rheum. 52:1020-1030 (2005). Radiographic progression (as measured by mean change in modified Sharp/van der Heijde score) was much greater in patients receiving MTX plus placebo than in patients receiving infliximab plus MTX. The authors conclude that even in patients without clinical improvement, treatment with infliximab plus MTX provided significant benefit with regard to the destructive process, suggesting that in such patients these two measures of disease are dissociated. The association between baseline radiographic damage and improvement in physical function after treatment of patients having RA with infliximab is described by Breedveld et al., Annals Rheumatic Diseases, 64:52-55 (2005). Structural damage was assessed using the van der Heijde modification of the Sharp score. The authors conclude that greater joint damage at baseline was associated with poorer physical function at baseline and less improvement in physical function after treatment, underlining the importance of early intervention to slow the progression of joint destruction.

### Autoimmune Disease Biomarkers

Autoantibodies are detected in a majority of patients with RA and predict more severe symptoms. The two major types of autoantibodies used clinically to create RA subsets are RF, which is an immunoglobulin specific to the Fc region of IgG, and anti-cyclic citrullinated peptide (CCP) antibodies. Anti-CCP recognizes proteins containing citrulline, which is the product of posttranslational modification of arginine residues. Masson-Bessiere et al., J Immunol., 166:4177-4184 (2001); Schellekens et al., Arthritis Rheum., 43:155-163 (2000). These autoantibodies are strongly correlated with RA, but may represent distinct clinical subsets thereof.

Szodoray et al., Scandinavian J. of Immunol., 60:209-218 (2004) discloses the apoptotic effect of rituximab on peripheral blood B cells in RA, with the data suggesting that rituximab is less effective in RF-negative RA because B cells play a less significant role in RA pathogenesis in RF-negative patients. US 2005/0271658 discloses that anti-CD20 antibodies can be used in a subject at risk for experiencing one or more symptoms of RA, and further wherein the subject has abnormal levels of IgM RF antibodies directed against the Fc portion of IgG. DiFranco et al., Rev. Rheum. Engl. Ed., 66(5):251-255 (1999) reported that quantitative RF isotype assays and magnetic resonance imaging evaluation of erosions of the hand and wrist may be useful for investigating patients with early RA. Ng et al., Ann Rheum. Dis., 66:1259 (2007) discloses that autoantibody profiling may help identity SLE patients who will have a more sustained response to B-cell depletion therapy with rituximab and cyclophosphamide, and whether baseline parameters can predict the likelihood of disease flare.

Anti-CCP antibodies are highly specific for RA, can be detected years before the first clinical manifestations of RA (Rantapaa-Dahlqvist et al, Arthritis Rheum., 48:2741-9 (2003)), and are reported to be a good predictor for the development of RA. Van Gaalen et al., Arthritis Rheum., 50:709-715 (2004). WO 2007/059188 discloses X-ray results regarding joint destruction in patients treated with anti-CD20 antibody. Tak et al. discloses the RF and anti-CCP markers in an abstract and poster entitled “Baseline autoantibody status (RF, Anti-CCP) and Clinical Response Following the First Treatment Course with Rituximab,” poster 833 at ACR 2006. This publication showed that patients who lacked both of these autoantibodies had a lower response rate to rituximab.

WO 2005/085858 discloses a method of assessing RA by measuring anti-CCP and serum amyloid A (SAA). WO 2005/064307 and US 2007/0264673 assess RA by measuring anti-CCP and IL-6. WO 2007/000169 discloses a non-human mammalian disease model to test diseases associated with anti-CCP such as arthritis, e.g., RA. US 2006/263355 discloses treatment of bone disorders using an anti-CD20 antibody, wherein the change in anti-CCP, CRP, S100, and SAA serum levels suggests that a single, short course with rituximab has a profound effect on markers. WO 2005/029091 and US 2006/094056 provide methods to diagnose, treat, or evaluate inflammatory/autoimmune diseases such as RA by sampling fluids from a human with a suspected diagnosis for certain cytokines. CN 1796997 notes a kit for early RA diagnosis by detecting anti-CCP. US 2007/0148704 and WO 2007/039280 disclose use of anti-CCP and antinuclear antibodies as biomarkers in diagnosing RA. WO 2006/008183 discloses various biomarkers for RA. U.S. Pat. No. 7,244,571 discloses a method for inducing a pro-asthma/pro-inflammatory-like state in a cell comprising contacting the cell with one or more cytokines. US 2007/0128626 discloses assessing response to anti-CD20 therapy by genotyping C1q components, e.g., the structure of the complement protein C1qA.

Scientific literature on anti-CCPs and/or RFs include Li et al., “Inferring causal relationships among intermediate phenotypes and biomarkers: a case study of rheumatoid arthritis” Bioinformatics, 22(12): 1503-1507 (2006); Russell et al., “The role of anti-cyclic citrullinated peptide antibodies in predicting progression of palindromic rheumatism to rheumatoid arthritis” J. Rheumatol., 33(7):1240-1242 (2006); Ota, “Immunologic laboratory testing in clinical practice for rheumatoid arthritis” Rinsho byori. Jap J. Clin. Pathol., 54(8):861-868 (2006); Avouac et al., “Diagnostic and predictive value of anti-cyclic citrullinated protein antibodies in rheumatoid arthritis: a systematic literature review” Ann. Rheum. Dis., 65(7):845-851 (2006); Mewar and Wilson, “Autoantibodies in rheumatoid arthritis: a review” Biomed. & Pharmacother., 60(10):648-655 (2006); Nielen et al., “Simultaneous development of acute phase response and autoantibodies in preclinical rheumatoid arthritis” Ann. Rheum. Dis., 65(4):535-537 (2006); Nielen et al. “Specific autoantibodies precede the symptoms of rheumatoid arthritis: a study of serial measurements in blood donors” Arthr. Rheum, 50:380-386 (2004)); Quinn et al., “Anti-CCP antibodies measured at disease onset help identify seronegative rheumatoid arthritis and predict radiological and functional outcome” Rheumatol., 45(4):478-480 (2006); Montano-Loza et al., “Frequency and significance of antibodies to cyclic citrullinated peptide in type 1 autoimmune hepatitis” Autoimmunity, 39(4):341-348 (2006); Griffiths, “Musculoskeletal disorders: an introduction” Medicine, 34(9):331-332 (2006); del Val del Amo et al., “Anti-cyclic citrullinated peptide antibody in rheumatoid arthritis: relation with disease aggressiveness” Clin. Exper. Rheum., 24(3):281-286 (2006); Schulze-Koops and Manger, “Diagnostic and prognostic significance of antibodies against citrullinated peptides” Deutsche Medizinische Wochenschrift (1946), 131(6):269-271 (2006); Matsui, “Antibodies to citrullinated proteins in rheumatoid arthritis” Jap. J. Clin. Immunol., 29(2):49-56 (2006); van Venrooij et al., “Autoantibodies to citrullinated antigens in (early) rheumatoid arthritis” Autoimmun. Rev., 6(1):37-41 (2006); Saleem et al., “Biomarkers: Strategies to predict outcome of rheumatoid arthritis” Drug Discovery Today: Therapeutic Strategies, 3(1): 11-16 (2006); Meyer et al., “Serial determination of cyclic citrullinated peptide autoantibodies predicted five-year radiological outcomes in a prospective cohort of patients with early rheumatoid arthritis” Arthr. Res. & Ther, 8(2):R40 (2006); Johansson et al., “PTPN22 polymorphism and anti-cyclic citrullinated peptide antibodies in combination strongly predicts future onset of rheumatoid arthritis and has a specificity of 100% for the disease” Arthr. Res. & Ther., 8(1):R19 (2006); de Seny et al., “Discovery of new rheumatoid arthritis biomarkers using the surface-enhanced laser desorption/ionization time-of-flight mass spectrometry ProteinChip approach” Arthr. & Rheum., 52(12):3801-3812 (2005); Radstake et al., “Correlation of rheumatoid arthritis severity with the genetic functional variants and circulating levels of macrophage migration inhibitory factor” Arthr. & Rheum., 52(10):3020-3029 (2005); Sihvonen et al., “The predictive value of rheumatoid factor isotypes, anti-cyclic citrullinated peptide antibodies, and antineutrophil cytoplasmic antibodies for mortality in patients with rheumatoid arthritis” J. Rheumat., 32(11):2089-2094 (2005); Nell et al., “Autoantibody profiling as early diagnostic and prognostic tool for rheumatoid arthritis” Ann. Rheum. Dis., 64(12):1731-1736 (2005); van Gaalen et al., “A comparison of the diagnostic accuracy and prognostic value of the first and second anti-cyclic citrullinated peptides (CCP1 and CCP2) autoantibody tests for rheumatoid arthritis” Ann. Rheum. Dis., 64(10): 1510-1512 (2005); Nielen et al., “Antibodies to citrullinated human fibrinogen (ACF) have diagnostic and prognostic value in early arthritis” Ann. Rheum. Dis., 64(8): 1199-1204 (2005); Mimori, “Clinical significance of anti-CCP antibodies in rheumatoid arthritis” Internal Medicine (Tokyo, Japan), 44(11): 1122-1126 (2005); Fusconi et al., “Anti-cyclic citrullinated peptide antibodies in type 1 autoimmune hepatitis” Alimentary Pharmacol. & Therapeut., 22(10):951-955 (2005); Hiura et al., “The examination of rheumatoid factor and other serum markers in rheumatoid arthritis” Yakugaku Zasshi, 125(11):881-887 (2005); Olivieri et al., “Management issues with elderly-onset rheumatoid arthritis: an update” Drugs & Aging, 22(10):809-822 (2005); Dai et al., “Significance of detecting anti-cyclic citrullinated peptide antibody in diagnosis of rheumatoid arthritis” Guangdong Yixue, 26(6):796-797 (2005); Yang et al., “Study on correlation between anti-cyclic citrullinated peptide antibody and erosion of bone in patients with rheumatoid arthritis” Huaxi Yixue 20(4): 658-660 (2005); Boire et al., “Anti-Sa antibodies and antibodies against cyclic citrullinated peptide are not equivalent as predictors of severe outcomes in patients with recent-onset polyarthritis” Arthr. Res. & Ther.,” 7(3):R592-603 (2005); Lienesch et al., “Absence of cyclic citrullinated peptide antibody in nonarthritic patients with chronic hepatitis C infection” J. Rheumatol., 32(3):489-493 (2005); Nakamura et al., “Clinical significance of anti-citrullinated peptide antibody in Japanese patients with established rheumatoid arthritis” Scandin. J. Rheumatol., 34(6): 489-490 (2005); Yang et al., “Clinical utility of EDRA/CPA in diagnosis of rheumatoid arthritis” Tianjin Yiyao 33(7):422-424 (2005); Momohara and Yamanaka, “Biomarker,” Igaku to Yakugaku, 53(4):413-425 (2005); Healy and Helliwell, “Classification of the spondyloarthropathies” Curr. Opin. Rheumatol., 17(4):395-399 (2005); Vallbracht and Helmke, “Additional diagnostic and clinical value of anti-cyclic citrullinated peptide antibodies compared with rheumatoid factor isotypes in rheumatoid arthritis” Autoimmun. Rev., 4(6):389-394 (2005); Kwok et al., “Anti-cyclic citrullinated peptide: diagnostic and prognostic values in juvenile idiopathic arthritis and rheumatoid arthritis in a Chinese population” Scandin. J. Rheumatol., 34(5):359-366 (2005); Senkpiehl et al., “HLA-DRB1 and Anti-Cyclic Citrullinated Peptide Antibody Production in Rheumatoid Arthritis” Intern. Arch. Allergy & Immunol., 137(4):315-318 (2005); Greiner et al., “Association of anti-cyclic citrullinated peptide antibodies, anti-citrulline antibodies, and IgM and IgA rheumatoid factors with serological parameters of disease activity in rheumatoid arthritis” Ann. NY Acad. Sci., 1050 (Autoim-munity):295-303 (2005); Raza et al., “Predictive value of antibodies to cyclic citrullinated peptide in patients with very early inflammatory arthritis” J. Rheum., 32(2):231-238 (2005); Tampoia et al., “Proteomic: new advances in the diagnosis of rheumatoid arthritis” Clinica Chimica Acta; Intern. J. Clin. Chem., 357 (2):219-225 (2005); van Leeuwen et al., “Prognostic significance of anti-CCP in early RA, relationship with shared epitope and rheumatoid factor” Annals of the Rheumatic Diseases, 64(Suppl. 3): 210 (2005); Annual European Congress of Rheumatology. Vienna, A T, Jun. 8-11, 2005; Chaiamnuay and Bridges, “The role of B cells and autoantibodies in rheumatoid arthritis” Pathophysiology: The Official Journal of the International Society for Pathophysiology/ISP, 12(3):203-216 (2005); Lindqvist et al., “Prognostic laboratory markers of joint damage in rheumatoid arthritis” Ann. Rheum. Dis., 64(2):196-201 (2005); Vencovsky et al., “Antibodies against citrullinated proteins in rheumatoid arthritis” Ceska Revmatologie, 13(4): 164-175 (2005); Egerer et al., “A new powerful marker for the diagnosis and prognosis of rheumatoid arthritis-Anti-CVM (Anti-Citrullinated vimentin mutated) antibodies” Arthr. & Rheum., 52(9, Suppl. S):S118 (2005); 69th Annual Scientific Meeting of the American-College-of-Rheumatology/40th Annual Scientific Meeting of the Association-of-Rheumatology-Health-Professionals, San Diego, Calif., Nov. 12-17, 2005; Kuribayashy et al., “Analysis of PADI4 gene polymorphisms in rheumatoid arthritis” J. Pharmacol. Sci., 97 (No. Suppl. 1):86P (2005); 78th Annual Meeting of the Japanese-Pharmacological-Society, Yokohama, J P, Mar. 22-24, 2005; Sedova et al., “Antibodies against cyclic citrullinated peptide (anti-CCP) in serum and synovial fluid from patients with rheumatoid arthritis and osteoarthritis” Ceska Revmatologie, 13(3):79-83 (2005); Boeckelmann et al., “Anti-cyclic citrullinated peptide antibodies occurring in psoriasis patients without arthritis” J. Invest. Derm., 125 (3, Suppl. S):A72 (2005); 35th Annual Meeting of the European-Society-for-Dermatological-Research, Tubingen, Del., Sep. 22-24, 2005; Dubrous et al., “Value of anti-cyclic citrullinated peptides antibodies in comparison with rheumatoid factor for rheumatoid arthritis diagnosis” Pathologie Biologie 53(2):63-67 (2005); Yamato et al., “Evaluation of basic properties of reagents for measuring anti-CCP (cyclic citrullinated peptide) antibody” Iryo to Kensa Kiki-Shiyaku 28(1):59-63 (2005); Vincent et al., “Autoantibodies to citrullinated proteins: ACPA” Autoimmunity, 38(1):17-24 (2005); Hitchon et al., “A distinct multicytokine profile is associated with anti-cyclical citrullinated peptide antibodies in patients with early untreated inflammatory arthritis” J. Rheumatol., 31 (12):2336-2346 (2004); Low et al., “Determination of anti-cyclic citrullinated peptide antibodies in the sera of patients with juvenile idiopathic arthritis” J. Rheumatol., 31(9):1829-1833 (2004); Forslind et al., “Prediction of radiological outcome in early rheumatoid arthritis in clinical practice: role of antibodies to citrullinated peptides (anti-CCP)” Ann. Rheum. Dis., 63(9):1090-1095 (2004); Kastbom et al., “Anti-CCP antibody test predicts the disease course during 3 years in early rheumatoid arthritis (the Swedish TIRA project),” Ann. Rheum. Dis., 63(9): 1085-1089 (2004); Vallbracht et al., “Diagnostic and clinical value of anti-cyclic citrullinated peptide antibodies compared with rheumatoid factor isotypes in rheumatoid arthritis” Ann. Rheum. Dis., 63(9): 1079-1084 (2004); Araki et al., “Usefulness of anti-cyclic citrullinated peptide antibodies (anti-CCP) for the diagnosis of rheumatoid arthritis” Rinsho Byori, 52(12):966-972 (2004); Kumagai et al., “Topics on immunological tests for rheumatoid arthritis” Rinsho byori. Jap. J. Clin. Pathol., 52(10):836-843 (2004); Eguchi, “Early diagnosis of rheumatoid arthritis by serological markers” Igaku no Ayumi, 209(10):802-808 (2004); Sawada, “New serum marker of rheumatoid arthritis” Gendai Iryo 36(3):718-722 (2004); van Gaalen et al., “Association between HLA class II genes and autoantibodies to cyclic citrullinated peptides (CCPs) influences the severity of rheumatoid arthritis.” Arthr. Rheum. 50(7):2113-2121 (2004); Sene et al., “Clinical utility of anti-cyclic citrullinated peptide antibodies in the diagnosis of hepatitis C virus associated-rheumatological manifestations” Hepatology, 40(4, Suppl. 1)-687A (2004); 55th Annual Meeting of the American-Association-for-the-Study-of-Liver-Diseases, Boston, Mass., Oct. 29-Nov. 2, 2004; Bongi et al., “Anti-cyclic citrullinated peptide antibodies are highly associated with severe bone lesions in rheumatoid arthritis anti-CCP and bone damage in RA” Autoimmunity, 37(6-7):495-501 (2004); Feng and Yin, “Detection of anti-cyclic citrullinated peptide antibodies in rheumatoid arthritis” Hebei Yike Daxue Xuebao, 25(6):371-373 (2004); Vossenaar and van Venrooij, “Anti-CCP antibodies, a highly specific marker for (early) rheumatoid arthritis” Clin. & Appl. Immunol. Rev., 4(4):239-262 (2004); Erre et al., “Diagnostic and prognostic value of antibodies to cyclic citrullinated peptide (Anti-CCP) in rheumatoid arthritis” Reumatismo, 56(2): 118-123 (2004); Bizzaro and Sebastiani “Laboratory diagnosis of rheumatoid arthritis” Progressi in Reumatologia, 5(1):82-88 (2004); Jansen et al., “The predictive value of anti-cyclic citrullinated peptide antibodies in early arthritis” J. Rheumatol., 30(8):1691-1695 (2003); Hayashi and Kumagai, “New diagnostic tests for rheumatoid arthritis” Rinsho Byori, 51(10):1030-1035 (2003); Salvador et al., “Prevalence and clinical significance of anti-cyclic citrullinated peptide and antikeratin antibodies in palindromic rheumatism. An abortive form of rheumatoid arthritis?” Rheumatology, 42(8):972-975 (2003); Okada and Kondo, “Early diagnosis and treatment of the bone and cartilage lesions in rheumatoid arthritis” Clinical Calcium, 13(6):729-733 (2003); Bas et al., “Anti-cyclic citrullinated peptide antibodies, IgM and IgA rheumatoid factors in the diagnosis and prognosis of rheumatoid arthritis” Rheumatology, 42 (Supplement 2):677-680 (2003); van Paassen et al., “Laboratory assessment in musculoskeletal disorders, Best practice & research” Clin. Rheumatol., 17(3):475-494 (2003); Vencovsky et al., “Autoantibodies can be prognostic markers of an erosive disease in early rheumatoid arthritis” Ann. Rheum. Dis., 62(5):427-430 (2003); Suzuki et al., “High diagnostic performance of ELISA detection of antibodies to citrullinated antigens in rheumatoid arthritis” Scand. J. Rheumatol., 32(4): 197-204 (2003); Vallbracht et al., “Additional diagnostic and clinical value of anti-citrullinated peptide antibodies in early rheumatoid arthritis compared to rheumatoid factor-isotypes” Ann. Rheum. Dis., 62 (No. Suppl. 1): 159 (2003); Annual European Congress of Rheumatology, Lisbon, P T, Jun. 18, 2003; Marcelletti and Nakamura, “Assessment of serological markers associated with rheumatoid arthritis. Diagnostic autoantibodies and conventional disease activity markers” Clin. Appl. Immunol. Rev., 4(2): 109-123 (2003); Hromadnikova et al., “Anti-cyclic citrullinated peptide antibodies in patients with juvenile idiopathic arthritis” Autoimmunity, 35(6):397-401 (2002); Vasishta, “Diagnosing early-onset rheumatoid arthritis: the role of anti-CCP antibodies” Amer. Clin. Lab., 21(7):34-36 (2002); Kroot et al., “The prognostic value of anti-cyclic citrullinated peptide antibody in patients with recent-onset rheumatoid arthritis” Arthr. & Rheum., 43(8):1831-1835 (2000); van Jaarsveld et al., “The prognostic value of the antiperinuclear factor, anti-citrullinated peptide antibodies and rheumatoid factor in early rheumatoid arthritis” Clin. & Exper. Rheumatol., 17(6):689-697 (1999); Kroot et al., “The prognostic value of the antiperinuclear factor, determined by a recently developed peptide-based ELISA, using anti citrulline-containing peptide antibodies (anti-CCP) in patients with recent onset Rheumatoid Arthritis” Arthr. & Rheum., 42 (9 Suppl.):S179 (1999). US 2007/0196835 discloses gene expression profiling for identification, monitoring, and treatment of RA. Ng et al., Ann Rheum. Dis., 66:1259 (2007) discloses that autoantibody profiling may help identity SLE patients who will have a more sustained response to B-cell depletion therapy with rituximab and cyclophosphamide, and whether baseline parameters can predict the likelihood of disease flare.

Anti-CCP antibodies are present in the majority of patients with RA within the first year of disease onset, further confirming the role of citrullinated proteins in the initiation of the immune dysregulation of RA. In fact, anti-CCP could be detected up to 2.6 years before the clinical onset of RA. Berglin et al., Arthr. Rheum., 48(9):S678 (2003). A study using the CCP2 assay (a second-generation assay) found progression from undifferentiated polyarthritis to RA in 93% of anti-CCP-positive patients but only in 25% of anti-CCP-negative patients after three years of follow-up. Jansen et al., J. Rheumatol., 29:2074-2076 (2002). A decrease in anti-CCP titers was also observed in RA patients treated with anti-TNF-α therapy in combination with low-dose MTX. Alessandri et al., Ann. Rheum. Dis., 63:1218-1221 (2004)). In this study, changes in anti-CCP titers and clinical responses were correlated; patients with best clinical improvement during the therapy had the lowest anti-CCP titers at baseline and showed strongest decrease in titer upon therapy. Anti-CCP, anti-keratin antibodies (AKA), and IgM RFs have been suggested as markers for RA. Bas et al., Rheumatology, 41(7):809-814 (2002). However, the value of such markers remains inconclusive. Scott, Rheumatology, 39/Suppl. 1:24-29 (2000). See also US 2006/263783. Citrulline is the essential antigenic epitope target of anti-perinuclear, anti-keratin, anti-filaggrin, anti-CCP, and anti-Sa antibodies. Van Venrooij and Pruijn, Arthritis Res., 2:249 (2000).

One important genetic risk factor for RA is the HLA-class II alleles within the MHC. Stastny and Fink, Transplant Proc., 9:1863-1866 (1977). These alleles are likely to contribute to about one-third of the genetic risk in RA. Deighton et al., Clin. Genet., 36:178-182 (1989); Rigby et al., Genet. Epidemiol., 8:153-175 (1991). Although the MHC associations with RA are complex (Jawaheer et al., Am. J. Hum. Genet., 71:585-594 (2002); Newton et al., Arthritis Rheum., 50:2122-2129 (2004)), the majority of the genetic signal from the MHC is explained by multiple alleles at the human leukocyte antigen HLA-DRB1 locus. Hall et al., QJM, 89:821-829 (1996); Jawaheer et al., supra, 2002; MacGregor et al., J. Rheumatol., 22:1032-1036 (1995). These alleles are known collectively as “shared epitope” (SE) alleles because of their sequence similarity at positions 70-74 within the third hypervariable region of the HLA-DRB1 alleles. Gregersen et al., Arthritis Rheum., 30:1205-1213 (1987)). SE haplotypes are associated with increased RA susceptibility risk. Also called rheumatoid epitope, SE can be found in approximately 80-90% of all Caucasian RA patients. However, most African-American patients with RA do not have the rheumatoid antigenic determinant (SE). McDaniel et al., Annals Int. Med., 123(3): 181-187 (1995).

It has been observed both by linkage and association analyses that the SE alleles are a risk factor for only RA characterized by the presence of anti-CCP antibodies, and not for anti-CCP-negative RA. Huizing a et al., Arthritis Rheum., 52:3433-3438 (2005). Van der Helm-van Mil et al., Arthritis and Rheum., 54:1117-1121 (2006) discloses that the SE-containing HLA-DRB1 alleles are primarily a risk factor for anti-CCP antibodies and are not an independent risk factor for development of RA.

PTPN22, also known as Lyp (see WO 1999/36548; Cohen et al., Immunobiology 93(6):2013-2024 (1999)), regulates the function of CbI and its associated protein kinases via its effect on the tyrosine protein kinase. Four proline-rich potential SH3-domain binding sites are located in the non-catalytic domain of PTNP22. PTPN22 regulates the function of Cb1 and its associated protein kinases. PTPN22 is an intracellular protein of about 105 kD with a single tyrosine phosphatase catalytic domain. Four proline-rich potential SH3 domain binding sites are located in the non-catalytic domain of PTPN22. PTNP22 is localized to chromosome lp13. PTPN22 has an alternative spliced isoform, Lyp2. Lyp2 is an 85-kD protein having a different seven-amino acid C-terminus. PTPN22 is expressed in a number of cell types involved in the immune response and inflammation. PTNP22 is highly expressed in lymphoid tissues and cells, including both mature B and T cells and thymocytes. Phytohemagglutinin induces PTPN22 expression in peripheral T lymphocytes. PTNP22 is also constitutively associated with the proto-oncogene c-Cbl in thymocytes and T cells. Cbl is a protein substrate of PTPN22, and is critical in the regulation of diverse processes in many cells and tissues. PTPN22 is expressed in myeloid cell lines as well as normal granulocytes and monocytes. PTPN22 is involved in CML. Erythroid and myeloid leukemic cell lines have distinct expression patterns of tyrosine phosphatases. In particular, the phosphorylation of multiple proteins in KCL22 chronic myeloid leukemia blast cells (e.g., CbI, Ber-Abl, Erkl/2, and CrkL PTPN221) is reduced by PTPN22 overexpression. Also, the phosphorylation of Bcr-Abl, Grb2, and Myc is reduced in Cos-7 cells co-expressing PTPN22 and Bcr-AbI. Also, anchorage-independent clonal growth of KCL22 cells is suppressed by PTPN22 overexpression. A negative regulatory role for Lyp in T-cell signaling is indicated by these interactions between Lyp and the adaptor Grb2. The ability of PTPN22 activity to reduce signaling by Bcr-Abl indicates PTPN22 is a potential tumor suppressor gene (Chien et al., J. Biol. Chem., 278:27413-27420 (2003)).

WO 2005/014622 discloses antigenic peptides binding to MHC Class II molecules with the SE referred to as HLA-DR molecules and the proteins from which they are derived as markers for erosive and/or non-erosive RA. The antigenic peptides can be used as markers in diagnosis of RA and in therapy as anti-RA vaccines. These include citrullinated antigenic peptides with an increased affinity for HLA-DR molecules and associated with RA. US 2006/062859 discloses methods to measure genetic and metabolic contributing factors affecting disease diagnosis, stratification, and prognosis, and the metabolism, efficacy, and/or toxicity associated with specific homeopathic ingredients. The DNA collected may be analyzed for polymorphisms of the Ras-Protein and HLA-DRB1 *0404 and *0101 or PTPN22 R620W and IL-10 genes, and the analysis may be used to adjust the dosage of Ganoderma Lucidum.

That PTPN22 has a functional role, with the mutation being associated with autoimmune risk and disease, is further illuminated by some of the literature discussed below.

WO 2006/010146 describes the human PTPN22 gene containing a single-nucleotide polymorphism (SNP) at nucleotide 1858 in codon 620, encoding an arginine in both alleles of the PTPN22 gene (PTPN22*R1 858) for the wild-type protein in all published human and mouse LYP sequences, but encodes a tryptophan in at least one allele of the PTPN22 gene (PTPN22*Tl 858), leading to a mutant LYP protein. The PTPN22*Tl858 allele predisposes a person to develop type 1 diabetes (T1D). The PTPN22 gene resides at chromosomal region lp13, linked to SLE and RA. The in vivo component of the screen can be the PTPN22 gene, or nucleotides 1858-1860 of the PTPN22 gene, or nucleotide 1858 of the PTPN22 gene. Or a genotyping assay can be used to determine the nucleotide present at position 1858 in the PTPN22 gene.

WO 2005/086872 describes methods for detecting polymorphisms of the PTPN22 genomic DNA; methods for associating polymorphisms of the PTPN22 gene with the occurrence of an immune disorder, inflammatory disorder, or cell proliferation disorder; methods for identifying subjects at risk of an immune disorder, inflammatory disorder, or cell-proliferation disorder by determining if they have a polymorphism of the PTPN22 gene, and treating such subjects with a tyrosine kinase inhibitor to prevent or delay the progression of such diseases; methods for identifying subjects having an immune disorder (e.g., RA), inflammatory disorder (e.g., Alzheimer's disease, arteriosclerosis), or cell-proliferation disorder (e.g., cancer, CML) who are promising candidates for therapy with a tyrosine kinase inhibitor by determining if such subjects have a polymorphism of the PTPN22 gene; and methods of treating subjects having such disorder mediated by a polymorphism of the PTPN22 gene by administering to such subjects a tyrosine kinase inhibitor. A SNP of the PTPN22 gene is determined in a nucleic acid sample obtained from the subject and the presence of the nucleotide occurrence is associated with reduced PTPN22 tyrosine phosphatase activity and altered phosphorylation of regulatory proteins and an increased incidence of the disorders above. A sample of tissue from the subject can be assayed for PTPN22 tyrosine phosphatase activity and the amount of such activity can determine if the subject would have increased risk for developing such disorder.

Feitsma et al., Rheumatology, 46:1092-1095 (2007) links anti-CCP titers with PTPN22 to predict progression of undifferentiated arthritis to RA.

U.S. Pat. No. 6,953,665 provides methods to classify an RA condition and to determine if a person suffering from an RA condition will develop severe disease. The method includes determining the level of a cytokine (e.g., IL-4, IL-10, and IFN-γ) within a patient sample, comparing the level of the cytokine to a reference level to obtain information about the RA condition, and classifying the RA condition as diffuse, follicular, or granulomatous. US 2005/266410 and WO 2005/123951 disclose approaches to mapping the MHC region and provide methods to genotype the HLA loci A haplotype map of the region and methods of using it. US 2003/232055 describes vaccines combining both signals needed to activate native T-cells—a specific antigen and the co-stimulatory signal—leading to a robust and specific T-cell immune response.

WO 2001/018240 notes a diagnostic method involving identifying a patient at risk of arthritis. The patient is tested to characterize a polymorphism in a first intron of the interferon-gamma gene. The polymorphisms may be distinguished based on a difference in the number of CA repeats in a portion of the first intron of the IFN-gamma gene. A patient may be tested for a polymorphism in an HLA protein (or gene), such as the HLA-DRB1 protein. WO 2001/012848 notes a method to determine the tendency of a person to develop RA and/or severity thereof, by detecting or measuring the presence of an FcγR gene, gene fragment, or gene product. U.S. Pat. No. 5,965,787 and WO 98/08943 disclose HLA-DRBI peptides with specific binding affinity for HLA-DQ molecules. Transgenic mice carrying a human HLA-DQ gene deficient in mouse H-2 class II molecules are models to identify peptides to prevent or treat RA. US 2003/099943 reports a method for detecting non-responders to anti-TNF therapy comprising testing a person for homozygosity for a SNP in the gene encoding the TNF receptor II. Anti-TNF-α (infliximab) represents a treatment for steroid-refractory Crohn's disease resulting in a remission rate of 30-50% after four weeks. Known SNPs within TNF Receptor I and TNF Receptor II were tested for association with response to therapy.

As for joint diseases, HLA-DRB1<SUP>0</SUP>0401, which is the allele of MHC, is reported to be associated with the development of chronic RA. Weyand et al., J. Clin. Invest., 89:2033-2039 (1992). See also the following on HLA, Fc receptor-like 3, MHC, and PTP mutations: Dieude and Cornelis, “Genetic basis of rheumatoid arthritis” Joint, Bone, Spine:Revue Du Rhumatisme, 72(6):520-526 (2005); Batliwalla et al., “Peripheral blood gene expression profiling in rheumatoid arthritis” Genes & Immunity, 6(5):388-397 (2005); Harrison et al., “Effects of PTPN22 C1858T polymorphism on susceptibility and clinical characteristics of British Caucasian rheumatoid arthritis patients” Rheumatology, 45(8): 1009-1011 (2006); Newman et al., Rheumatoid arthritis association with the FCRL3-169C polymorphism is restricted to PTPN22 1858T-homozygous individuals in a Canadian population” Arthr & Rheum., 54(12):3820-3827 (2006); Barcellos et al., “Clustering of autoimmune diseases in families with a high-risk for multiple sclerosis: a descriptive study” Lancet Neurology, 5(11):924-931 (2006); Ikari et al., “Haplotype analysis revealed no association between the PTPN22 gene and RA in a Japanese population” Rheumatology, 45(11): 1345-1348 (2006); Wipff et al., “Lack of association between the protein tyrosine phosphatase non-receptor 22 (PTPN22)*620W allele and systemic sclerosis in the French Caucasian population” Ann. Rheum. Dis., 65(9):1230-1232 (2006); Ray et al., “Protein tyrosine phosphatase non-receptor type 22 (PTPN22) gene R620W variant and sporadic idiopathic hypoparathyroidism in Asian Indians” Intern. J. Immunogen., 33(4):237-240 (2006); Harrison et al., “Effects of PTPN22 C1858T polymorphism on susceptibility and clinical characteristics of British Caucasian rheumatoid arthritis patients” Rheumatology, 45(8): 1009-1011 (2006); Pierer et al., “Association of PTPN22 1858 single-nucleotide polymorphism with rheumatoid arthritis in a German cohort: higher frequency of the risk allele in male compared to female patients” Arthr. Res. & Ther., 8(3):R75 (2006); Butt et al., “Association of functional variants of PTPN22 and tp53 in psoriatic arthritis: a case-control study” Arthr. Res. & Ther, 8(1):R27 (2006); Bottini et al., “Role of PTPN22 in type 1 diabetes and other autoimmune diseases” Seminars in Immunology, 18(4):207-213 (2006); Smyth et al., “Analysis of polymorphisms in 16 genes in type 1 diabetes that have been associated with other immune-mediated diseases” BMC Medical Genetics, 7:20 (2006); De Jager et al., Evaluating the role of the 620W allele of protein tyrosine phosphatase PTPN22 in Crohn's disease and multiple sclerosis” European J. Hum. Gen., 14(3):317-321 (2006); Oliver et al., “Genetic epidemiology of rheumatoid arthritis” Curr. Opin. Rheumatol., 18(2): 141-146 (2006); Burkhardt et al., “Association between protein tyrosine phosphatase 22 variant R620W in conjunction with the HLA-DRB1 shared epitope and humoral autoimmunity to an immunodominant epitope of cartilage-specific type II collagen in early rheumatoid arthritis” Arthr. & Rheum., 54(1):82-89 (2006); Jagiello et al., “The PTPN22 620W allele is a risk factor for Wegener's granulomatosis” Arthr. & Rheum., 52(12):4039-4043 (2005); Vang et al., “Autoimmune-associated lymphoid tyrosine phosphatase is a gain-of-function variant” Nature Genetics, 37(12):1317-1319 (2005); Worthington, “Investigating the genetic basis of susceptibility to rheumatoid arthritis” J. Autoimmunity, 25 Suppl: 16-20 (2005); Dieude et al., “Rheumatoid arthritis seropositive for the rheumatoid factor is linked to the protein tyrosine phosphatase nonreceptor 22-620W allele” Arthr. Res. & Ther., 7(6):R1200-1207 (2005); Gomez et al., “PTPN22 C1858T polymorphism in Colombian patients with autoimmune diseases” Genes & Immun., 6(7):628-631 (2005); Wesoly et al., Association of the PTPN22 C1858T single-nucleotide polymorphism with rheumatoid arthritis phenotypes in an inception cohort” Arthritis & Rheum., 52(9):2948-2950 (2005); Prescott et al., “A general autoimmunity gene (PTPN22) is not associated with inflammatory bowel disease in a British population” Tissue Antigens, 66(4):318-320 (2005); Carlton et al., “PTPN22 genetic variation: evidence for multiple variants associated with rheumatoid arthritis” Amer. J. Hum. Gen., 77(4):567-581 (2005); Zhernakova et al., “Differential association of the PTPN22 coding variant with autoimmune diseases in a Dutch population” Genes & Immunity, 6(6):459-461 (2005); Hinks et al., “Association between the PTPN22 gene and rheumatoid arthritis and juvenile idiopathic arthritis in a UK population: further support that PTPN22 is an autoimmunity gene” Arthr. & Rheum., 52(6): 1694-1699 (2005); Simkins et al., “Association of the PTPN22 locus with rheumatoid arthritis in a New Zealand Caucasian cohort” Arthr. &Rheum., 52(7):2222-2225 (2005); Gregersen and Batliwalla, PTPN22 and rheumatoid arthritis: gratifying replication” Arthr. & Rheum., 52(7): 1952-1955 (2005); van Oene et al., “Association of the lymphoid tyrosine phosphatase R620W variant with rheumatoid arthritis, but not Crohn's disease, in Canadian populations” Arthr. & Rheumat., 52(7): 1993-1998 (2005); Mori et al., “Ethnic differences in allele frequency of autoimmune-disease-associated SNPs” J. Human Gen., 50(5):264-266 (2005); Viken et al., “Association analysis of the 1858C>T polymorphism in the PTPN22 gene in juvenile idiopathic arthritis and other autoimmune diseases” Genes & Immunity, 6(3):271-273 (2005); van der Helm-van Mil et al., “Understanding the genetic contribution to rheumatoid arthritis” Curr. Opinion Rheumatol., 17(3):299-304 (2005); Gregersen, “Pathways to gene identification in rheumatoid arthritis: PTPN22 and beyond” Immunological Rev., 204:74-86 (2005); Criswell et al., “Analysis of families in the multiple autoimmune disease genetics consortium (MADGC) collection: the PTPN22 620W allele associates with multiple autoimmune phenotypes” Amer. J. Hum. Gen., 76(4):561-571 (2005); Zheng and She, “Genetic association between a lymphoid tyrosine phosphatase (PTPN22) and type 1 diabetes” Diabetes, 54(3):906-908 (2005); Ladner et al., “Association of the single nucleotide polymorphism C1858T of the PTPN22 gene with type 1 diabetes” Human Immunol., 66(1):60-64 (2005); Orozco et al., “Association of a functional single-nucleotide polymorphism of PTPN22, encoding lymphoid protein phosphatase, with rheumatoid arthritis and systemic lupus erythematosus” Arthr. & Rheum., 52(1):219-224 (2005); Brenner et al., “The non-major histocompatibility complex quantitative trait locus Cia10 contains a major arthritis gene and regulates disease severity, pannus formation, and joint damage” Arthr. & Rheum., 52(1):322-332 (2005); Begovich et al., “A missense single-nucleotide polymorphism in a gene encoding a protein tyrosine phosphatase (PTPN22) is associated with rheumatoid arthritis” Amer. J. Hum. Gen., 75(2):330-337 (2004); Reveille, “The genetic basis of autoantibody production” Autoimmun. Rev., 5(6):389-398 (2006); Mustelin “Allelic variation in signaling elements and autoimmunity” Seminars in Immunology, 18(4): 197-198 (2006); Brand et al., “HLA, CTLA-4 and PTPN22: The shared genetic master-key to autoimmunity?” Expert Rev. in Molec. Med., 7(23):1-15 (2005); Vandiedonck et al., “Association of the PTPN22*R620W polymorphism with autoimmune myasthenia gravis” Ann. Neurol., 59(2):404-407 (2006); Pearce and Merriman, “Genetic progress towards the molecular basis of autoimmunity” Trends in Molec. Med., 12(2): 90-98 (2006); Gregersen, “Gaining insight into PTPN22 and autoimmunity” Nat. Gen., 37(12): 1300-1302 (2005); Gregersen and Batliwalla, “PTPN22 and rheumatoid arthritis: Gratifying replication” Arthr. & Rheum., 52(7): 1952-1955 (2005); Alarcon-Riquelme, “The genetics of shared autoimmunity” Autoimmunity, 38(3):205-208 (2005); Steer et al., “Association of R602W in a protein tyrosine phosphatase gene with a high risk of rheumatoid arthritis in a British population: Evidence for an early onset/disease severity effect” Arthr. & Rheum., 52(1):358-360 (2005); Hueffmeier et al., “Male restricted genetic association of variant R620W in PTPN22 with psoriatic arthritis” J. Invest. Derm., 126(4):936-938 (2006); Anonymous, “1st Mexican-Canadian Congress of Rheumatology, Acapulco, MEXICO, Feb. 17-21, 2006” J. Rheumatol., 33(2):405-428 (2006); Orozco et al., “Association of a functional single nucleotide polymorphism of PTPN22 with rheumatoid arthritis and systemic lupus erythematosus” Genes and Immunity, 6(Suppl. 1): S32 (April 2005); Butt et al., “Association of functional variants of Ptpn22 and Tp53 PsA in Caucasian population” Arth. & Rheum., 52(9, Suppl. S):S642 (September 2005); Wyeth et al., Association analysis of rheumatoid arthritis candidate susceptibility genes in New Zealand Maori” Arthr. & Rheum., 52(9, Suppl. S):S582 (September 2005); Gomez et al., “Polymorphism in gene coding for LYP is a risk factor for primary Sjögren's syndrome and systemic lupus erythematosus” Arthr. & Rheum., 52(9, Suppl. S):S376 (September 2005); Burkhardt et al., “Protein tyrosine phosphatase 22 variant R620W in conjunction with HLA-DRB1 shared epitope is associated with humoral autoimmunity to an immunodominant epitope of cartilage-specific type II collagen in early rheumatoid arthritis” Arthr. & Rheum., 52(9, Suppl. S):S146 (September 2005); Harrison et al., “The PTPN22 R620W polymorphism-Effects on susceptibility and clinical features on British Caucasian rheumatoid arthritis patients” Arthr. & Rheum., 52(9, Suppl. S):S145-S146 (September 2005); Costenbader et al., “The PTPN22 polymorphism and the risk of rheumatoid arthritis: Results from the Nurses' Health Study” Arthr. & Rheum., 52(9, Suppl. S):S145 (September 2005); Wesoly et al., “PTPN22 1858T allele as rheumatoid arthritis susceptibility but not severity gene variant” Annals Rheumatic Diseases, 64:(No. Suppl. 3):78 (July 2005); Dieude et al., “The protein tyrosine phosphatase R620W polymorphism is linked and associated with rheumatoid arthritis seropositive for the rheumatoid factor in a caucasian population” Ann. Rheum. Dis., 64(No. Suppl. 3):78 (July 2005); Anonymous, Joint Meeting of the British-Society-for-Rheumatology/Deutsche-Gesellschaft-fur-Rheumatologie and Spring Meeting of the British-Health-Professionals-in-Rheumatology, Birmingham, ENGLAND, Apr. 19-22, 2005, Rheumatology, 44(Suppl. 1):I2-I164 (March 2005); Matesanz et al., “Protein tyrosine phosphatase gene (PTPN22) polymorphism in multiple sclerosis” J. Neurol., 252(8): 994-995 (2005); Lee et al., “The PTPN22 C1858T functional polymorphism and autoimmune diseases-a meta-analysis” Rheumatology, 46(1):49-56 (2007); Gregersen and Plenge, “Emerging relationships: rheumatoid arthritis and the PTPN22 associated autoimmune disorders” in Hereditary Basis of Rheumatic Diseases, Ed.: Holmdahl, (Birkhaeuser Verlag, Basel, C H, 2006), pp. 61-78; van der Helm-van Mil and Huizing a, “Genetics and clinical characteristics to predict rheumatoid arthritis: where are we now and what are the future prospects?” Future Rheumatology 1(1):79-89 (2006); Hueffmeier et al., “Male restricted genetic association of variant R620W in PTPN22 with psoriatic arthritis” J. Invest. Dermatol., 126(4):932-935 (2006); Yamada, “Large scale SNP LD mapping of rheumatoid arthritis-associated genes” Rinsho Men'eki 44(4):406-410 (2005); Kochi, “Recent findings on rheumatoid arthritis genetics” Igaku no Ayumi 215(4):259-260 (2005); Yamamoto et al., “Rheumatoid arthritis as multifactorial genetic diseases” Saishin Igaku 60(9, Zokango, Rinsho Idenshigaku '05):2111-2119 (2005); Yamada, “Rheumatoid arthritis-associated genes,” Saishin Igaku 60(9): 1935-1939 (2005); Velazquez-Cruz et al., “A Functional SNP of PTPN22 is Associated with Childhood-Onset Systemic Lupus Erythematosus, but not with Juvenile Rheumatoid Arthritis in Mexican Population” 11th Intern. Cong. of Human Genetics (ICHG 2006), Brisbane Convention and Exhibition Centre, Brisbane, Queensland (Australia), 6-10 Aug. 2006, Prof. Lyn Griffiths, Griffith University, Brisbane.

The non-synonymous SNP (R620W) in the PTPN22 gene is associated with increased susceptibility risk to RA, juvenile idiopathic arthritis, SLE, Addison's disease, systemic sclerosis, Grave's disease, and type 1 diabetes. See, for example, Plenge et al., Am. J. Hum. Genet. 77:1044-1060 (2005) reporting that the R620W variant of PTPN22 is associated with the development of RF-positive and anti-CCP-positive RA and stating that the results provide support for an association of RA with variants in PAD14 and CTLA4. See also Plenge and Rioux, Immunol. Rev., 210:40-51 (2006) on identifying susceptibility genes for immunological disorders. Further, Lee et al., Genes and Immunity, 6:129-133 (2004) discloses that the PTPN22 R620W polymorphism associates with RF-positive RA in a dose-dependent manner, but not with HLA-SE status. Seldin et al., Genes and Immunity, 6:720-722 (2005) discloses evidence that PTPN22 R620W polymorphism is a risk factor in RA, but suggests only minimal or no effect in juvenile idiopathic arthritis. Hinks et al., Rheumatology 45(4):365-368 (2006) discloses the association of PTPN22 with RA and juvenile idiopathic arthritis. See also the editorial in Rheumatology, 45:365-368 (2006) on the association of PTPN22 with RA and juvenile idiopathic arthritis. Hinks, Future Rheumatology, 1:153-158 (2006) explores whether PTPN22 is a confirmed RA susceptibility gene.

Kyogoku et al., The American Journal of Human Genetics, 75:504-507 (2004) discloses the genetic association of the R620W polymorphism of PTPN22 with human SLE. Kaufman et al., Arthritis Rheum., 54:2533-40 (2006) reports that the 1858T allele of PTPN22 is associated with familial SLE but not with sporadic SLE in European Americans, thereby potentially explaining previous contradictory reports. Wu et al., Arthritis and Rheumatism, 52:2396-2402 (2005) reports on the association analysis of the R620W polymorphism of PTPN22 in SLE families, specifically the increased t allele frequency in SLE patients with autoimmune thyroid disease.

Gourh et al., Arthritis Rheum., 54(12):3945-3953 (December 2006) discloses an association of the PTPN22 R620W polymorphism with anti-topoisomerase I- and anti-centromere antibody-positive systemic sclerosis. However, Begovich et al., Am J Hum Genet., 76(1):184-187 (2005) discloses that the R620W polymorphism of PTPN22 is not associated with MS. Gomez et al., Human Immunology, 66:1242-1247 (2005) discloses the genetic influence of PTPN22 R620W polymorphism in tuberculosis. Qu et al., J. Medical Genetics 42:266-270 (2005) reports the confirmation of the association of the R620W polymorphism in PTPN22 with type 1 diabetes in a family-based study. Nistor et al., J. Invest. Dermatol., pp. 395-396 (Letter to the Editor) (2005) discloses lack of evidence for association of the PTPN22 polymorphism with psoriasis. See also Nistor et al., “Protein tyrosine phosphatase gene PTPN22 polymorphism is not associated with psoriasis” J. Invest. Derm., 124(4, Suppl. S):A80 (April 2005).

Wagenleiter et al., Inter. J. Immunogen., 32 (5):323-324 (2005) discloses a case-control study of PTPN22 confirming the lack of association with Crohn's disease. Martín et al., Tissue Antigens 66 (4):314-317 (2005) discloses that the functional genetic variation in the PTPN22 gene has a negligible effect on the susceptibility to develop inflammatory bowel disease.

TNF-α, IL-1β, and IL-1Ra gene polymorphisms are associated with increased RA susceptibility risk and disease severity. Paradowska and Lacki, Centr Eur J Immunol., 31(3-4): 117-122 (2006). IL-1 and TNF-α gene polymorphisms are associated with levels of anti-cytokine, including anti-TNF, clinical responses. WO 2001/000880 and EP 1172444.

The FcγRIIa (Val/Phe 158) and FcγRIIa (His/Arg 131) polymorphisms predicted rituximab clinical response in follicular lymphoma. The FcγRIIa (His/Arg 131) polymorphism predicted B-cell depletion efficacy in SLE. The FcγRIIb (−343 G/C) polymorphism is associated with increased SLE susceptibility. A review summarizes how FcγRIIb expression may influence the anti-tumor immune reaction and how beneficial or deleterious this expression could be for the efficiency of therapeutics based on monoclonal anti-tumor antibodies, including rituximab. Cassard et al., Springer Seminars in Immunopathology, 28(4):321-328 (2006).

See also “Clinical Response Following the First Treatment Course with Rituximab: Effect of Baseline Autoantibody Status (RF, Anti-CCP)” Ann. Rheumatic Diseases, 66(Suppl. 2):338 (July 2007).

A method of assessing RA by analyzing biochemical markers is disclosed in US 2007/0072237 involving measuring in a sample the concentration of RF and IL-6 and correlating the concentrations determined to the absence or presence of RA. The level of one or more additional markers may be determined together with RF and IL-6 and may be correlated to the absence or presence of RA.

### B-Cell Related Disclosure

Lymphocytes are one of many types of white blood cells produced in the bone marrow during the process of hematopoiesis. There are two major populations of lymphocytes: B lymphocytes (B cells) and T lymphocytes (T cells). The lymphocytes of particular interest herein are B cells.

B cells mature within the bone marrow and leave the marrow expressing an antigen-binding antibody on their cell surface. When a naïve B cell first encounters the antigen for which its membrane-bound antibody is specific, the cell begins to divide rapidly and its progeny differentiate into memory B cells and effector cells called “plasma cells.” Memory B cells have a longer life span and continue to express membrane-bound antibody with the same specificity as the original parent cell. Plasma cells do not produce membrane-bound antibody, but instead produce the antibody in a form that can be secreted. Secreted antibodies are the major effector molecules of humoral immunity.

B-cell-related disorders include autoimmune diseases. Cytotoxic agents that target B-cell surface antigens are an important focus of B-cell-related cancer therapies. One such B-cell surface antigen is CD20, disclosed more in detail below. Other B-cell antigens, such as CD19, CD22, and CD52, represent targets of therapeutic potential for treatment of lymphoma. Grillo-Lopez et al., Curr. Pharm. Biotechnol., 2:301-311 (2001). CD22 is a 135-kDa B-cell-restricted sialoglycoprotein expressed on the B-cell surface only at the mature stages of differentiation. Dorken et al., J. Immunol., 136:4470-4479 (1986). The predominant form of CD22 in humans is CD22beta, which contains seven immunoglobulin superfamily domains in the extracellular domain. Wilson et al., J. Exp. Med., 173:137-146 (1991). A variant form, CD22alpha, lacks immunoglobulin superfamily domains 3 and 4. Stamenkovic and Seed, Nature, 345:74-77 (1990). Ligand-binding to human CD22 has been shown to be associated with immunoglobulin superfamily domains 1 and 2 (also referred to as epitopes 1 and 2). Engel et al., J. Exp. Med., 181:1581-1586 (1995).

In B-cell NHL, CD22 expression ranges from 91% to 99% in the aggressive and indolent populations, respectively. Cesano et al., Blood, 100:350a (2002). CD22 may function both as a component of the B-cell activation complex (Sato et al., Semin. Immunol., 10:287-296 (1998)) and as an adhesion molecule. Engel et al., J. Immunol., 150:4719-4732 (1993). The B cells of CD22-deficient mice have a shorter life span and enhanced apoptosis, which suggests a key role of this antigen in B-cell survival. Otipoby et al., Nature, 384:634-637 (1996). After binding with its natural ligand(s) or antibodies, CD22 is rapidly internalized, providing a potent costimulatory signal in primary B cells and proapoptotic signals in neoplastic B cells. Sato et al., Immunity, 5:551-562 (1996).

Anti-CD22 antibodies have been studied as potential therapies for B-cell cancers and other B-cell proliferative diseases. Such anti-CD22 antibodies include RFB4 (Mansfield et al., Blood, 90:2020-2026 (1997)), CMC-544 (DiJoseph, Blood, 103:1807-1814 (2004)), and LL2 (Pawlak-Byczkowska et al., Cancer Res., 49:4568-4577 (1989)). The LL2 antibody (formerly called HPB-2) is an IgG2a mouse monoclonal antibody directed against the CD22 antigen. Pawlak-Byczkowska et al., 1989, supra. In vitro immunohistological evaluations demonstrated reactivity of the LL2 antibody with 50 of 51 B-cell NHL specimens tested, but not with other malignancies or normal non-lymphoid tissues. Pawlak-Byczkowska et al., 1989, supra; Stein et al., Cancer Immunol. Immunother., 37:293-298 (1993).

The CD20 antigen (also called human B-lymphocyte-restricted differentiation antigen, Bp35, or B1) is a four-pass, glycosylated integral membrane protein with a molecular weight of approximately 35 kD located on pre-B and mature B lymphocytes. Valentine et al., J. Biol. Chem., 264(19):11282-11287 (1989); Einfeld et al., EMBO J., 7(3):711-717 (1988). The antigen is also expressed on greater than 90% of B-cell non-Hodgkin's lymphomas (NHL) (Anderson et al., Blood, 63(6): 1424-1433 (1984)), but is not found on hematopoietic stem cells, pro-B cells, normal plasma cells, or other normal tissues (Tedder et al., J. Immunol., 135(2):973-979 (1985)). CD20 regulates an early step(s) in the activation process for cell-cycle initiation and differentiation (Tedder et al., supra), and possibly functions as a calcium-ion channel. Tedder et al., J. Cell. Biochem., 14D: 195 (1990). CD20 undergoes phosphorylation in activated B cells. Riley and Sliwkowski, Semin. Oncol., 27(12): 17-24 (2000). CD20 appears on the surface of B-lymphocytes at the pre-B-cell stage and is found on mature and memory B cells, but not plasma cells. Stashenko et al., J. Immunol., 125:1678-1685 (1980); Clark and Ledbetter, Adv. Cancer Res., 52:81-149 (1989). CD20 has calcium-channel activity and may play a role in the development of B cells. Rituximab displays antibody-dependent cellular cytotoxicity (ADCC) in vitro. Reff et al., Blood, 83:435-445 (1994). Potent complement-dependent cytotoxic (CDC) activity has also been observed for rituximab in lymphoma cells and cell lines (Reff et al., supra, 1994) and in certain mouse xenograft models. DiGaetano et al., J. Immunol., 171:1581-1587 (2003). Several anti-CD20 antibodies, including rituximab, have been shown to induce apoptosis in vitro when crosslinked by a secondary antibody or by other means. Ghetie et al., Proc. Natl. Acad. Sci. USA, 94:7509-7514 (1997).

Given the expression of CD20 in B-cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD 20 antibody such that the agent is specifically “delivered” to the neoplastic B cells. Irrespective of the approach, a primary goal is to destroy the tumor; the specific approach can be determined by the particular anti-CD20 antibody that is utilized, and thus, the available approaches to targeting the CD20 antigen can vary considerably.

The rituximab (RITUXAN®) antibody is a genetically engineered chimeric murine/human monoclonal antibody directed against the CD20 antigen. Rituximab is the antibody called “C2B8” in U.S. Pat. No. 5,736,137 (Anderson et al.). Rituximab is indicated for the treatment of patients with relapsed or refractory low-grade or follicular, CD20-positive, B-cell non-Hodgkin's lymphoma. In vitro mechanism-of-action studies have demonstrated that rituximab binds human complement and lyses lymphoid B-cell lines through CDC. Reff et al., Blood, 83(2):435-445 (1994). Additionally, it has significant activity in assays for ADCC. Rituximab has been shown to have anti-proliferative effects in tritiated thymidine-incorporation assays and to induce apoptosis directly, while other anti-CD19 and anti-CD20 antibodies do not. Maloney et al., Blood, 88(10):637a (1996). Synergy between rituximab and chemotherapies and toxins has also been observed experimentally. In particular, rituximab sensitizes drug-resistant human B-cell lymphoma cell lines to the cytotoxic effects of doxorubicin, CDDP, VP-16, diphtheria toxin, and ricin. Demidem et al., Cancer Chemotherapy & Radiopharmaceuticals, 12(3):177-186 (1997). In vivo preclinical studies have shown that rituximab depletes B cells from the peripheral blood, lymph nodes, and bone marrow of cynomolgus monkeys, presumably through complement- and cell-mediated processes. Reff et al., Blood, 83:435-445 (1994).

Rituximab was approved in the United States for the treatment of patients with relapsed or refractory low-grade or follicular CD20⁺B-cell NHL at a dose of 375 mg/m² weekly for four doses. In April 2001, the Food and Drug Administration (FDA) approved additional claims for the treatment of low-grade NHL: re-treatment (weekly for four doses) and an additional dosing regimen (weekly for eight doses). Many patients have been exposed to rituximab either as monotherapy or in combination with immunosuppressant or chemotherapeutic drugs. Patients have also been treated with rituximab as maintenance therapy for up to two years. Hainsworth et al., J. Clin. Oncol., 21:1746-1751 (2003); Hainsworth et al., J. Clin. Oncol., 20:4261-4267 (2002). Also, rituximab has been used in the treatment of malignant and nonmalignant plasma cell disorders. Treon and Anderson, Semin. Oncol., 27: 79-85 (2000).

Rituximab has also been approved in the United States in combination with MTX to reduce signs and symptoms in adult patients with moderately- to severely-active RA who have had an inadequate response to at least one TNF antagonist. Many studies address the use of rituximab in a variety of non-malignant autoimmune disorders, including RA, in which B cells and autoantibodies appear to play a role in disease pathophysiology. Edwards et al., Biochem Soc. Trans. 30:824-828 (2002). Rituximab has been reported to potentially relieve signs and symptoms of, for example, RA (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): S439 (2003)), lupus (Eisenberg, Arthritis. Res. Ther. 5:157-159 (2003); Leandro et al. Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaja et al., Haematologica, 87:189-195 (2002); Ratanatharathorn et al., Ann. Int. Med., 133:275-279 (2000)), pure red cell aplasia (Auner et al., Br. J. Haematol., 116:725-728 (2002)); autoimmune anemia (Zaja et al., supra (erratum appears in Haematologica 87:336 (2002)), cold agglutinin disease (Layios et al., Leukemia, 15:187-8 (2001); Berentsen et al., Blood, 103: 2925-2928 (2004); Berentsen et al., Br. J. Haematol., 115:79-83 (2001); Bauduer, Br. J. Haematol., 112:1083-1090 (2001); Zaja et al., Br. J. Haematol., 115:232-233 (2001)), type B syndrome of severe insulin resistance (Coll et al., N. Engl. J. Med., 350:310-311 (2004), mixed cryoglobulinermia (DeVita et al., Arthritis Rheum. 46 Suppl. 9:S206/S469 (2002)), myasthenia gravis (Zaja et al., Neurology, 55:1062-1063 (2000); Wylam et al., J. Pediatr., 143:674-677 (2003)), Wegener's granulomatosis (Specks et al., Arthritis & Rheumatism 44:2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjogren's syndrome (Somer et al., Arthritis & Rheumatism, 49:394-398 (2003)), active type-II mixed cryoglobulinemia (Zaja et al., Blood, 101:3827-3834 (2003)), pemphigus vulgaris (Dupay et al., Arch. Dermatol., 140:91-95 (2004)), autoimmune neuropathy (Pestronk et al., J. Neurol. Neurosurg. Psychiatry 74:485-489 (2003)), paraneoplastic opsoclonus-myoclonus syndrome (Pranzatelli et al. Neurology 60(Suppl. 1) PO5.128:A395 (2003)), and relapsing-remitting multiple sclerosis (RRMS). Cross et al. (abstract) “Preliminary Results from a Phase II Trial of Rituximab in MS” Eighth Annual Meeting of the Americas Committees for Research and Treatment in Multiple Sclerosis, 20-21 (2003).

A Phase II study (WA16291) has been conducted in patients with RA, providing 48-week follow-up data on safety and efficacy of rituximab. Emery et al. Arthritis Rheum 48(9):S439 (2003); Szczepanski et al. Arthritis Rheum 48(9):S121 (2003). A total of 161 patients were evenly randomized to four treatment arms: methotrexate, rituximab alone, rituximab plus methotrexate, and rituximab plus cyclophosphamide (CTX). The treatment regimen of rituximab was one gram administered intravenously on days 1 and 15. Infusions of rituximab in most patients with RA were well tolerated by most patients, with 36% of patients experiencing at least one adverse event during their first infusion (compared with 30% of patients receiving placebo). Overall, the majority of adverse events was considered to be mild to moderate in severity and was well balanced across all treatment groups. There were a total of 19 serious adverse events across the four arms over the 48 weeks, which were slightly more frequent in the rituximab/CTX group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study. Doran et al., Arthritis Rheum. 46:2287-2293 (2002).

The reported safety profile of rituximab in a small number of patients with neurologic disorders, including autoimmune neuropathy (Pestronk et al., supra), opsoclonus-myoclonus syndrome (Pranzatelli et al., supra), and RRMS (Cross et al., supra), was similar to that reported in oncology or RA. In an investigator-sponsored trial (IST) of rituximab combined with interferon-beta (IFN-β) or glatiramer acetate in patients with RRMS (Cross et al., supra), one of ten treated patients was admitted to the hospital for overnight observation after experiencing moderate fever and rigors following the first infusion of rituximab, while the other nine patients completed the four-infusion regimen without any reported adverse events.

Patents and patent publications concerning CD20 antibodies, CD20-binding molecules, and self-antigen vaccines include U.S. Pat. Nos. 5,776,456, 5,736,137, 5,843,439, 6,399,061, and 6,682,734, as well as US 2002/0197255, US 2003/0021781, US 2003/0082172, US 2003/0095963, US 2003/0147885, US 2005/0186205, and WO 1994/11026 (Anderson et al.); U.S. Pat. No. 6,455,043, US 2003/0026804, US 2003/0206903, and WO 2000/09160 (Grillo-Lopez, A.); WO 2000/27428 (Grillo-Lopez and White); US 2004/0213784 and WO 2000/27433 (Grillo-Lopez and Leonard); WO 2000/44788 (Braslawsky et al.); WO 2001/10462 (Rastetter, W.); WO 2001/10461 (Rastetter and White); WO 2001/10460 (White and Grillo-Lopez); US 2001/0018041, US 2003/0180292, US 2002/0028178, WO 2001/34194, and WO 2002/22212 (Hanna and Hariharan); US 2002/0006404 and WO 2002/04021 (Hanna and Hariharan); US 2002/0012665, US 2005/0180975, WO 2001/74388, and U.S. Pat. No. 6,896,885B5 (Hanna, N.); US 2002/0058029 (Hanna, N.); US 2003/0103971 (Hariharan and Hanna); US 2005/0123540 (Hanna et al.); US 2002/0009444 and WO 2001/80884 (Grillo-Lopez, A.); WO 2001/97858; US 2005/0112060, US 2002/0039557, and U.S. Pat. No. 6,846,476 (White, C.); US 2002/0128448 and WO 2002/34790 (Reff, M.); WO 2002/060955 (Braslawsky et al.); WO 2002/096948 (Braslawsky et al.); WO 2002/079255 (Reff and Davies); U.S. Pat. Nos. 6,171,586 and 6,991,790, and WO 1998/56418 (Lam et al.); US 2004/0191256 and WO 1998/58964 (Raju, S.); WO 1999/22764 (Raju, S.); WO 1999/51642, U.S. Pat. No. 6,194,551, U.S. Pat. No. 6,242,195, U.S. Pat. No. 6,528,624 and U.S. Pat. No. 6,538,124 (Idusogie et al.); U.S. Pat. No. 7,122,637, US 2005/0118174, US 2005/0233382, US 2006/0194291, US 2006/0194290, US 2006/0194957, and WO 2000/42072 (Presta, L.); WO 2000/67796 (Curd et al.); WO 2001/03734 (Grillo-Lopez et al.); US 2002/0004587, US 2006/0025576, and WO 2001/77342 (Miller and Presta); US 2002/0197256 and WO 2002/078766 (Grewal, I.); US 2003/0157108 and WO 2003/035835 (Presta, L.); U.S. Pat. Nos. 5,648,267, 5,733,779, 6,017,733, and 6,159,730, and WO 1994/11523 (Reff et al. on expression technology); U.S. Pat. Nos. 6,565,827, 6,090,365, 6,287,537, 6,015,542, 5,843,398, and 5,595,721 (Kaminski et al.); U.S. Pat. Nos. 5,500,362, 5,677,180, 5,721,108, 6,120,767, 6,652,852, and 6,893,625 as well as WO 1988/04936 (Robinson et al.); U.S. Pat. No. 6,410,391 (Zelsacher); U.S. Pat. No. 6,224,866 and WO00/20864 (Barbera-Guillem, E.); WO 2001/13945 (Barbera-Guillem, E.); WO 2000/67795 (Goldenberg); U.S. Pat. No. 7,074,403 (Goldenberg and Hansen); U.S. Pat. No. 7,151,164 (Hansen et al.); US 2003/0133930; WO 2000/74718 and US 2005/0191300A1 (Goldenberg and Hansen); US 2003/0219433 and WO 2003/68821 (Hansen et al.); WO 2004/058298 (Goldenberg and Hansen); WO 2000/76542 (Golay et al.); WO 2001/72333 (Wolin and Rosenblatt); U.S. Pat. No. 6,368,596 (Ghetie et al.); U.S. Pat. No. 6,306,393 and US 2002/0041847 (Goldenberg, D.); US 2003/0026801 (Weiner and Hartmann); WO 2002/102312 (Engleman, E.); US 2003/0068664 (Albitar et al.); WO 2003/002607 (Leung, S.); WO 2003/049694, US 2002/0009427, and US 2003/0185796 (Wolin et al.); WO 2003/061694 (Sing and Siegall); US 2003/0219818 (Bohen et al.); US 2003/0219433 and WO 2003/068821 (Hansen et al.); US 2003/0219818 (Bohen et al.); US 2002/0136719 (Shenoy et al.); WO 2004/032828 and US 2005/0180972 (Wahl et al.); and WO 2002/56910 (Hayden-Ledbetter). See also U.S. Pat. No. 5,849,898 and EP 330,191 (Seed et al.); EP332,865A2 (Meyer and Weiss); U.S. Pat. No. 4,861,579 (Meyer et al.); US 2001/0056066 (Bugelski et al.); WO 1995/03770 (Bhat et al.); US 2003/0219433 A1 (Hansen et al.); WO 2004/035607 and US 2004/167319 (Teeling et al.); WO 2005/103081 (Teeling et al.); US 2006/0034835, US 2006/0024300, and WO 2004/056312 (Lowman et al.); US 2004/0093621 (Shitara et al.); WO 2004/103404 (Watkins et al.); WO 2005/000901 (Tedder et al.); US 2005/0025764 (Watkins et al.); US 2006/0251652 (Watkins et al.); WO 2005/016969 (Carr et al.); US 2005/0069545 (Carr et al.); WO 2005/014618 (Chang et al.); US 2005/0079174 (Barbera-Guillem and Nelson); US 2005/0106108 (Leung and Hansen); US 2005/0123546 (Umana et al.); US 2004/0072290 (Umana et al.); US 2003/0175884 (Umana et al.); and WO 2005/044859 (Umana et al.); WO 2005/070963 (Allan et al.); US 2005/0186216 (Ledbetter and Hayden-Ledbetter); US 2005/0202534 (Hayden-Ledbetter and Ledbetter); US 2005/136049 (Ledbetter et al.); US 2003/118592 (Ledbetter et al.); US 2003/133939 (Ledbetter and Hayden-Ledbetter); US 2005/0202012 (Ledbetter and Hayden-Ledbetter); US 2005/0175614 (Ledbetter and Hayden-Ledbetter); US 2005/0180970 (Ledbetter and Hayden-Ledbetter); US 2005/0202028 (Hayden-Ledbetter and Ledbetter); US 2005/0202023 (Hayden-Ledbetter and Ledbetter); WO 2005/017148 (Ledbetter et al.); WO 2005/037989 (Ledbetter et al.); U.S. Pat. No. 6,183,744 (Goldenberg); U.S. Pat. No. 6,897,044 (Braslawski et al.); WO 2006/005477 (Krause et al.); US 2006/0029543 (Krause et al.); US 2006/0018900 (McCormick et al.); US 2006/0051349 (Goldenberg and Hansen); WO 2006/042240 (Iyer and Dunussi-Joannopoulos); US 2006/0121032 (Dahiyat et al.); WO 2006/064121 (Teillaud et al.); US 2006/0153838 (Watkins), CN 1718587 (Chen et al.); WO 2006/084264 (Adams et al.); US 2006/0188495 (Barron et al.); US 2004/0202658 and WO 2004/091657 (Benynes, K.); US 2005/0095243, US 2005/0163775, WO 2005/00351, and WO 2006/068867 (Chan, A.); US 2006/0135430 and WO 2005/005462 (Chan et al.); US 2005/0032130 and WO 2005/017529 (Beresini et al.); US 2005/0053602 and WO 2005/023302 (Brunetta, P.); US 2006/0179501 and WO 2004/060052 (Chan et al.); WO 2004/060053 (Chan et al.); US 2005/0186206 and WO 2005/060999 (Brunetta, P.); US 2005/0191297 and WO 2005/061542 (Brunetta, P.); US 2006/0002930 and WO 2005/115453 (Brunetta et al.); US 2006/0099662 and WO 2005/108989 (Chuntharapai et al.); CN 1420129A (Zhongxin Guojian Pharmaceutical); US 2005/0276803 and WO 2005/113003 (Chan et al.); US 2005/0271658 and WO 2005/117972 (Brunetta et al.); US 2005/0255527 and WO 2005/11428 (Yang, J.); US 2006/0024295 and WO 2005/120437 (Brunetta, P.); US 2006/0051345 and WO 2005/117978 (Frohna, P.); US 2006/0062787 and WO 2006/012508 (Hitraya, E.); US 2006/0067930 and WO 2006/31370 (Lowman et al.); WO 2006/29224 (Ashkenazi, A.); US 2006/0110387 and WO 2006/41680 (Brunetta, P.); US 2006/0134111 and WO 2006/066086 (Agarwal, S.); WO 2006/069403 (Ernst and Yansura); US 2006/0188495 and WO 2006/076651 (Dummer, W.); WO 2006/084264 (Lowman, H.); WO 2006/093923 (Quan and Sewell); WO 2006/106959 (Numazaki et al.); WO 2006/126069 (Morawala); WO 2006/130458 (Gazit-Bornstein et al.); US 2006/0275284 (Hanna, G.); US 2007/0014785 (Golay et al.); US 2007/0014720 (Gazit-Bornstein et al.); and US 2007/0020259 (Hansen et al.); US 2007/0020265 (Goldenberg and Hansen); US 2007/0014797 (Hitraya); US 2007/0224189 (Lazar et al.); and WO 2008/003319 (Parren and Baadsgaard).

Scientific publications concerning treatment with rituximab include: Perotta and Abuel, “Response of chronic relapsing ITP of 10 years duration to rituximab” Abstract #3360 Blood, 10(1)(part 1-2):88B (1998); Perotta et al., “Rituxan in the treatment of chronic idiopathic thrombocytopaenic purpura (ITP)”, Blood, 94:49 (abstract) (1999); Matthews, R., “Medical Heretics” New Scientist, (7 Apr., 2001); Leandro et al., “Clinical outcome in 22 patients with rheumatoid arthritis treated with B lymphocyte depletion” Ann Rheum Dis., supra; Leandro et al., “Lymphocyte depletion in rheumatoid arthritis: early evidence for safety, efficacy and dose response” Arthritis and Rheumatism, 44(9):S370 (2001); Leandro et al., “An open study of B lymphocyte depletion in systemic lupus erythematosus” Arthritis and Rheumatism, 46:2673-2677 (2002), wherein during a two-week period, each patient received two 500-mg infusions of rituximab, two 750-mg infusions of cyclophosphamide, and high-dose oral corticosteroids, and wherein two of the patients treated relapsed at seven and eight months, respectively, and have been retreated, although with different protocols; “Successful long-term treatment of systemic lupus erythematosus with rituximab maintenance therapy” Weide et al., Lupus, 12:779-782 (2003), wherein a patient was treated with rituximab (375 mg/m²×4, repeated at weekly intervals) and further rituximab applications were delivered every five to six months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes” Rheumatology, 40:205-211 (2001); Cambridge et al., “B lymphocyte depletion in patients with rheumatoid arthritis: serial studies of immunological parameters” Arthritis Rheum., 46 (Suppl. 9): S1350 (2002); Cambridge et al., “Serologic changes following B lymphocyte depletion therapy for rheumatoid arthritis” Arthritis Rheum., 48:2146-2154 (2003); Edwards et al., “B-lymphocyte depletion therapy in rheumatoid arthritis and other autoimmune disorders” Biochem Soc. Trans., supra; Edwards et al., “Efficacy and safety of rituximab, a B-cell targeted chimeric monoclonal antibody: A randomized, placebo controlled trial in patients with rheumatoid arthritis. Arthritis and Rheumatism, 46(9):S197 (2002); Edwards et al., “Efficacy of B-cell-targeted therapy with rituximab in patients with rheumatoid arthritis” N Engl. J. Med., 350:2572-2582 (2004); Pavelka et al., Ann. Rheum. Dis., 63:(S1):289-290 (2004); Emery et al., Arthritis Rheum. 50 (S9):S659 (2004); Levine and Pestronk, “IgM antibody-related polyneuropathies: B-cell depletion chemotherapy using Rituximab” Neurology, 52:1701-1704 (1999); Uchida et al., “The innate mononuclear phagocyte network depletes B lymphocytes through Fc receptor-dependent mechanisms during anti-CD20 antibody immunotherapy” J. Exp. Med., 199:1659-1669 (2004); Gong et al., “Importance of cellular microenvironment and circulatory dynamics in B cell immunotherapy” J. Immunol., 174:817-826 (2005); Hamaguchi et al., “The peritoneal cavity provides a protective niche for B1 and conventional B lymphocytes during anti-CD20 immunotherapy in mice” J. Immunol., 174:4389-4399 (2005); Cragg et al. “The biology of CD20 and its potential as a target for mAb therapy” Curr. Dir. Autoimmun., 8:140-174 (2005); Eisenberg, “Mechanisms of autoimmunity” Immunol. Res., 27:203-218 (2003); DeVita et al., “Efficacy of selective B cell blockade in the treatment of rheumatoid arthritis” Arthritis & Rheum, 46:2029-2033 (2002); Higashida et al. “Treatment of DMARD-refractory rheumatoid arthritis with rituximab” Annual Scientific Meeting of the American College of Rheumatology (Abstract #LB11), New Orleans, La. (October, 2002); Tuscano, “Successful treatment of infliximab-refractory rheumatoid arthritis with rituximab” Annual Scientific Meeting of the American College of Rheumatology, New Orleans, La. (October, 2002), published as Tuscano, Arthritis Rheum. 46:3420 (2002); “Pathogenic roles of B cells in human autoimmunity; insights from the clinic” Martin and Chan, Immunity, 20:517-527 (2004); Silverman and Weisman, “Rituximab therapy and autoimmune disorders, prospects for anti-B cell therapy”, Arthritis and Rheumatism, 48:1484-1492 (2003); Kazkaz and Isenberg, “Anti B cell therapy (rituximab) in the treatment of autoimmune diseases” Current opinion in pharmacology, 4:398-402 (2004); Virgolini and Vanda, “Rituximab in autoimmune diseases” Biomedicine & pharmacotherapy, 58: 299-309 (2004); Klemmer et al., “Treatment of antibody mediated autoimmune disorders with a AntiCD20 monoclonal antibody Rituximab” Arthritis And Rheumatism, 48(9) (SEP):S624-S624 (2003); Kneitz et al., “Effective B cell depletion with rituximab in the treatment of autoimmune diseases” Immunobiology, 206:519-527 (2002); Arzoo et al., “Treatment of refractory antibody mediated autoimmune disorders with an anti-CD 20 monoclonal antibody (rituximab)” Annals of the Rheumatic Diseases, 61(10):922-924 (2002) Comment in Ann Rheum Dis. 61:863-866 (2002); “Future strategies in immunotherapy” by Lake and Dionne, in Burger's Medicinal Chemistry and Drug Discovery (John Wiley & Sons, Inc., 2003) (Chapter 2 “Antibody-Directed Immunotherapy”); Liang and Tedder, Wiley Encyclopedia of Molecular Medicine, Section: CD20 as an Immunotherapy Target (2002); Appendix 4A entitled “Monoclonal Antibodies to Human Cell Surface Antigens” by Stockinger et al., eds: Coligan et al., in Current Protocols in Immunology (John Wiley & Sons, Inc., 2003); Penichet and Morrison, “CD Antibodies/molecules: Definition; Antibody Engineering” in Wiley Encyclopedia of Molecular Medicine Section: Chimeric, Humanized and Human Antibodies (2002).

Further, see Looney “B cells as a therapeutic target in autoimmune diseases other than rheumatoid arthritis” Rheumatology, 44 Suppl 2:ii13-ii17 (2005); Chambers and Isenberg, “Anti-B cell therapy (rituximab) in the treatment of autoimmune diseases” Lupus, 14(3):210-214 (2005); Looney et al., “B-cell depletion as a novel treatment for systemic lupus erythematosus: a phase I/II dose-escalating trial of rituximab” Arthritis Rheum., 50:2580-2589 (2004); Looney, “Treating human autoimmune disease by depleting B cells” Ann Rheum. Dis., 61:863-866 (2002); Edelbauer et al., “Rituximab in childhood systemic lupus erythematosus refractory to conventional immunosuppression Case report” Pediatr. Nephrol., 20(6): 811-813 (2005); D'Cruz and Hughes, “The treatment of lupus nephritis” BMJ, 330(7488):377-378 (2005); Looney, “B cell-targeted therapy in diseases other than rheumatoid arthritis” J. Rheumatol. Suppl., 73: 25-28-discussion 29-30 (2005); Sfikakis et al., “Remission of proliferative lupus nephritis following B cell depletion therapy is preceded by down-regulation of the T cell costimulatory molecule CD40 ligand: an open-label trial” Arthritis Rheum., 52(2):501-513 (2005); Rastetter et al., “Rituximab: expanding role in therapy for lymphomas and autoimmune diseases” Annu. Rev. Med., 55:477-503 (2004); Silverman, “Anti-CD20 therapy in systemic lupus erythematosus: a step closer to the clinic” Arthritis Rheum., 52(2):371-377 (2005), Erratum in: Arthritis Rheum. 52(4):1342 (2005); Ahn et al., “Long-term remission from life-threatening hypercoagulable state associated with lupus anticoagulant (LA) following rituximab therapy” Am. J. Hematol., 78(2): 127-129 (2005); Tahir et al., “Humanized anti-CD20 monoclonal antibody in the treatment of severe resistant systemic lupus erythematosus in a patient with antibodies against rituximab” Rheumatology, 44(4):561-562 (2005), Epub 2005, Jan. 11; Looney et al., “Treatment of SLE with anti CD20 monoclonal antibody” Curr. Dir. Autoimmun., 8:193-205 (2005); Cragg et al., “The biology of CD20 and its potential as a target for mAb therapy” Curr. Dir. Autoimmun., 8:140-174 (2005); Gottenberg et al., “Tolerance and short term efficacy of rituximab in 43 patients with systemic autoimmune diseases” Ann. Rheum. Dis., 64(6):913-920 (2005) Epub 2004 Nov. 18; Tokunaga et al., “Down-regulation of CD40 and CD80 on B cells in patients with life-threatening systemic lupus erythematosus after successful treatment with rituximab” Rheumatology 44(2): 176-182 (2005), Epub 2004 Oct. 19. See also Leandro et al., “B cell repopulation occurs mainly from naïve B cells in patient with rheumatoid arthritis and systemic lupus erythematosus” Arthritis Rheum., 48 (Suppl 9): S1160 (2003).

Specks et al. “Response of Wegener's granulomatosis to anti-CD20 chimeric monoclonal antibody therapy” Arthritis & Rheumatism, 44(12):2836-2840 (2001) disclosed successful use of four infusions of 375 mg/m² of rituximab and high-dose glucocorticoids to treat Wegener's granulomatosis. The therapy was repeated after 11 months when the cANCA recurred, but therapy was without glucocorticoids. At eight months after the second course of rituximab, the patients' disease remained in complete remission. In another study rituximab was found to be a well-tolerated, effective remission induction agent for severe ANCA-associated vasculitis, when used in a dose of 375 mg/m²×four along with oral prednisone at 1 mg/kg/day, which was reduced to 40 mg/day by week four, and to total discontinuation over the following 16 weeks. Four patients were re-treated with rituximab alone for recurring/rising ANCA titers. Other than glucocorticoids, no additional immunosuppressive agents seem necessary for remission induction and maintenance of sustained remission (six months or longer). Keogh et al., Kidney Blood Press. Res., 26:293 (2003) reported that eleven patients with refractory ANCA-associated vasculitis went into remission upon treatment with four weekly 375 mg/m² doses of rituximab and high-dose glucocorticoids.

Patients with refractory ANCA-associated vasculitis were administered rituximab along with immunosuppressive medicaments such as intravenous cyclophosphamide, mycophenolate mofetil, azathioprine, or leflunomide, with apparent efficacy. Eriksson, “Short-term outcome and safety in 5 patients with ANCA-positive vasculitis treated with rituximab” Kidney and Blood Pressure Research, 26:294 (2003) (five patients with ANCA-associated vasculitis treated with rituximab 375 mg/m² once a week for four weeks responded to the treatment); Jayne et al., “B-cell depletion with rituximab for refractory vasculitis” Kidney and Blood Pressure Research, 26:294-295 (2003) (six patients with refractory vasculitis receiving four weekly infusions of rituximab at 375 mg/m² with cyclophosphamide along with background immunosuppression and prednisolone experienced major falls in vasculitic activity). A further report of using rituximab along with intravenous cyclophosphamide at 375 mg/m² per dose in four doses for administering to patients with refractory systemic vasculitis is provided in Smith and Jayne, “A prospective, open label trial of B-cell depletion with rituximab in refractory systemic vasculitis” poster 998 (11^{th} International Vasculitis and ANCA workshop), American Society of Nephrology, J. Am. Soc. Nephrol., 14:755A (2003). See also Eriksson, J. Internal Med., 257:540-548 (2005) regarding nine patients with ANCA-positive vasculitis who were successfully treated with two or four weekly doses of 500 mg of rituximab; and Keogh et al., Arthritis and Rheumatism, 52:262-268 (2005), who reported that in 11 patients with refractory ANCA-associated vasculitis, treatment or re-treatment with four weekly 375 mg/m² doses of rituximab induced remission by B-lymphocyte depletion (study conducted from January 2000 to September 2002).

As to the activity of a humanized anti-CD20 antibody, see, for example, Vugmeyster et al., “Depletion of B cells by a humanized anti-CD20 antibody PRO70769 in Macaca fascicularis,” J. Immunother., 28:212-219 (2005). For discussion of a human monoclonal antibody, see Baker et al., “Generation and characterization of LymphoStat-B, a human monoclonal antibody that antagonizes the bioactivities of B lymphocyte stimulator,” Arthritis Rheum., 48:3253-3265 (2003). The MINT trial with rituximab was successful in treating aggressive non-Hodgkin's lymphoma in younger patients. Pfreundschuh et al., Lancet Oncology, 7(5):379-391 (2006).

BLyS™ (also known as BAFF, TALL-1, THANK, TNFSF13B, or zTNF4) is a member of the TNF1 ligand superfamily that is essential for B-cell survival and maturation. BAFF overexpression in transgenic mice leads to B-cell hyperplasia and development of severe autoimmune disease. Mackay et al., J. Exp. Med., 190:1697-1710 (1999); Gross et al., Nature, 404:995-999 (2000); Khare et al., Proc. Natl. Acad. Sci. U.S.A, 97:3370-3375 (2000). BAFF levels are elevated in human patients with a variety of autoimmune disorders, such as SLE, RA, and Sjögren's syndrome. Cheema et al., Arthritis Rheum., 44:1313-1319 (2001); Groom et al, J. Clin. Invest., 109:59-68 (2002); Zhang et al., J. Immunol., 166:6-10 (2001). Furthermore, BAFF levels correlate with disease severity, suggesting that BAFF can play a direct role in the pathogenesis of these illnesses. BAFF acts on B cells by binding to three members of the TNF receptor superfamily, TACI, BCMA, and BR3 (also known as BAFF-R). Gross et al., supra; Thompson et al., Science, 293:2108-2111 (2001); Yan et al., Curr. Biol. 11:1547-1552 (2001); Yan et al., Nat. Immunol., 1:37-41 (2000); Schiemann et al., Science, 293:2111-2114 (2001).

Of the three, only BR3 is specific for BAFF; the other two also bind the related TNF family member, A proliferation-inducing ligand (APRIL). Comparison of the phenotypes of BAFF and receptor knockout or mutant mice indicates that signaling through BR3 mediates the B-cell survival functions of BAFF. Thompson et al., supra; Yan et al., supra, 2001; Schiemann et al., supra. In contrast, TACI appears to act as an inhibitory receptor (Yan, Nat. Immunol., 2:638-643 (2001)), while the role of BCMA is unclear. Schiemann et al., supra. US 2007/0071760 discloses treating B-cell malignancies using a TACI-Ig fusion molecule in an amount sufficient to suppress proliferation-inducing functions of BlyS and APRIL.

BR3 is a 184-residue type III transmembrane protein expressed on the surface of B cells. Thompson et al., supra; Yan, Nat. Immun., supra. The intracellular region bears no sequence similarity to known structural domains or protein-protein interaction motifs. Nevertheless, BAFF-induced signaling through BR3 results in processing of the transcription factor NF-B2/p100 to p52. Claudio et al., Nat. Immunol., 3:958-965 (2002); Kayagaki et al., Immunity, 10:515-524 (2002). The extracellular domain (ECD) of BR3 is also divergent. TNFR family members are usually characterized by the presence of multiple cysteine-rich domains (CRDs) in their extracellular region; each CRD is typically composed of about 40 residues stabilized by six cysteines in three disulfide bonds. Conventional members of this family make contacts with ligand through two CRDs interacting with two distinct patches on the ligand surface. Bodmer et al., Trends Biochem. Sci., 27:19-26 (2002). However, the BR3ECD contains only four cysteine residues, capable of forming a partial CRD at most, raising the question of how such a small receptor imparts high-affinity ligand binding.

It has been shown that the BAFF-binding domain of BR3 resides within a 26-residue core region. Kayagaki et al., supra. Six BR3 residues, when structured within a β-hairpin peptide (bhpBR3), were sufficient to confer BAFF binding and block BR3-mediated signaling. Others have reported polypeptides purported to interact with BAFF (e.g., WO 2002/24909, WO 2003/035846, WO 2002/16312, and WO 2002/02641).

For any given RA patient one frequently cannot predict if he/she is likely to respond to a particular treatment, even with newer B-cell antagonist therapies. This necessitates considerable trial and error, often at significant risk and discomfort to the patient, to find the most effective therapy.

Thus, there is a need for more effective means to determine which patients will respond to which treatment and for incorporating such determinations into more effective treatment regimens for RA patients with B-cell antagonist therapies, whether used as single agents or combined with other agents to treat RA.

### SUMMARY OF THE INVENTION

The present invention concerns the recognition that patients with RA can be selected for B-cell antagonist therapy based on the presence of certain diagnostic indicators in a sample taken from the patient. The present invention provides diagnostic methods for predicting the effectiveness of treatment of a RA patient with a B-cell antagonist directed against B-cell surface markers or B-cell specific proliferation or survival factors. In particular, the invention concerns prediction of the efficacy response to RA therapy with a B-cell antagonist based on the incidence of specified genetic markers (shared epitope (SE) and/or PTPN22 R620W polymorphism) alone or in combination with expression of other biomarkers, particularly RF and/or anti-CCP autoantibody reactivity. Biomarker sets can be built from any combination of suitable biomarkers that includes the PTPN22 R620W polymorphism or SE or both. The invention is as claimed.

Accordingly, in one particular aspect, the invention provides a method of treating RA in a patient comprising administering an effective amount of a B-cell antagonist to the patient to treat the RA, provided that a PTPN22 R620W SNP or SE or both SNP and SE is/are present in a genetic sample from the patient (e.g., a nucleic acid sample).

In another embodiment, the invention provides use of a B-cell antagonist in the manufacture of a pharmaceutical composition (or a medicament) for treating RA, provided that a PTPN22 R620W SNP or SE or both SNP and SE is/are present in a genetic sample from a patient being treated for RA.

Also, the invention provides a method of treating RA in a patient comprising administering to the patient an effective amount of a B-cell antagonist, wherein before the administration, expression of PTNP22 R620W SNP, or SE, or both the SNP and SE was detected in a genetic sample (such as a biological sample) from the patient.

Further provided is a method of treating RA in a patient comprising administering to the patient an effective amount of a B-cell antagonist, wherein before the administration a genetic sample from the patient was determined to exhibit expression of PTNP22 R620W SNP, or SE, or both the SNP and SE, whereby the expression indicates that the patient will respond to treatment with the antagonist.

Also provided is a method of treating RA in a patient comprising administering to the patient an effective amount of a B-cell antagonist, wherein before the administration a genetic sample from the patient was determined to exhibit expression of PTNP22 R620W SNP, or SE, or both the SNP and SE, whereby the expression indicates that the patient is likely to respond favorably to treatment with the antagonist.

In a preferred embodiment, expression of the SNP, but not the SE, is assessed. In another embodiment, expression of the SE, but not the SNP, is assessed. In another preferred aspect, expression of both the SNP and SE is assessed.

In one aspect of these methods, samples from the patient do not reveal any biomarker indicating responsiveness of the patient to B-cell antagonist treatment other than the SNP or shared epitope or both. Thus, the expression of the SNP and/or SE is assessed not in combination with another biomarker.

In another aspect of these methods, samples from the patient do reveal one or more biomarkers indicating responsiveness of the patient to B-cell antagonist treatment other than the SNP or shared epitope or both. Thus, the expression of the SNP and/or SE is assessed in combination with other biomarkers. In a preferred aspect of this embodiment a sample from the patient is seropositive for one or both of the additional biomarkers anti-CCP antibody and RF.

Thus, the invention resides in the assessment of PTPN22 R620W SNP and/or SE expression alone or, optionally, in one embodiment, in combination with seropositivity for an autoantibody such as RF and/or anti-CCP antibody.

In one preferred aspect, the additional biomarker is anti-CCP antibody, preferably of the IgG or IgM isotype. In another preferred aspect, the additional biomarker is a RF, more preferably with an IgA, IgG, or IgM isotype. In another preferred aspect, the additional biomarkers are both anti-CCP antibody and RF. In a particularly preferred aspect, expression of SE is assessed along with seropositivity for RF, without assessment of the SNP or anti-CCP antibody, i.e., the SE is present along with seropositivity for RF, without the presence of the SNP or anti-CCP antibody. In another especially preferred aspect, the SNP is present along with seropositivity for anti-CCP antibody, without presence of the SE or RF.

In another aspect, preferably, the antagonist is an antibody or immunoadhesin. In another preferred aspect the antagonist is directed against a specific B-cell proliferative or survival factor, such as BAFF or APRIL. Examples of preferred BAFF antagonists include anti-BR3 antibodies and BR3-Fc. Examples of preferred APRIL antagonists include atacicept (same as TACI-Ig immunoadhesin) and a BAFF/APRIL antagonist (soluble BCMA-Fc). In another aspect, the antagonist is an antibody, more preferably a chimeric, humanized, or human antibody. Most preferably, the antagonist is anti-CD20 antibody, anti-CD 22 antibody, anti-BR3 antibody, BR3-Fc, or TACI-Ig. In a still further aspect, the antagonist is to CD20, CD22, BAFF, or APRIL.

In one particularly preferred embodiment, the antagonist is anti-CD20 or anti-CD 22 antibody, more preferably anti-CD20 antibody, still more preferably rituximab or a 2H7 antibody. More preferably, the 2H7 antibody comprises the L-chain variable region sequence of SEQ ID NO:1 and the H-chain variable region sequence of SEQ ID NO:2, or comprises the L-chain variable region sequence of SEQ ID NO:3 and the H-chain variable region sequence of SEQ ID NO:4, or comprises the L-chain variable region sequence of SEQ ID NO:3 and the H-chain variable region sequence of SEQ ID NO:5, or comprises the full-length L chain of SEQ ID NO:6 and the full-length H chain of SEQ ID NO:7, or comprises the full-length L chain of SEQ ID NO:6 and the full-length H chain of SEQ ID NO:8, or comprises the full-length L chain of SEQ ID NO:9 and the full-length H chain of SEQ ID NO:10, or comprises the full-length L chain of SEQ ID NO:9 and the full-length H chain of SEQ ID NO:11, or comprises the full-length L chain of SEQ ID NO:9 and the full-length H chain of SEQ ID NO:12, or comprises the full-length L chain of SEQ ID NO:9 and the full-length H chain of SEQ ID NO:13, or comprises the full-length L chain of SEQ ID NO:9 and the full-length H chain of SEQ ID NO:14, or comprises the full-length L chain of SEQ ID NO:6 and the full-length H chain of SEQ ID NO:15.

In another embodiment, the antagonist is not conjugated with a cytotoxic agent.

In an alternative embodiment, it is conjugated with a cytotoxic agent.

In another preferred aspect of these methods, the patient has never been previously administered a medicament for the RA, or for any autoimmune disease.

In another aspect, the patient has been previously administered at least one medicament for the RA or for any autoimmune disorder. In a further embodiment, the patient was not responsive to at least one medicament that was previously administered, with exemplary such previously administered medicament or medicaments selected from the group consisting of an immunosuppressive agent, cytokine antagonist, integrin antagonist, corticosteroid, analgesic, disease-modifying anti-rheumatic drug (DMARD), and non-steroidal anti-inflammatory drug (NSAID). More preferably, the patient was not responsive to at least one immunosuppressive agent, cytokine antagonist, integrin antagonist, corticosteroid, DMARD, or NSAID, especially not responsive to MTX or a TNF-α inhibitor. In an alternative preferable embodiment, the patient was not responsive to at least one B-cell antagonist, such as anti-CD20 antibody, preferably an antagonist that is not rituximab or a 2H7 antibody. In another aspect, the patient was not responsive to rituximab or a 2H7 antibody.

In other preferred aspects, the antagonist is administered intravenously or subcutaneously, most preferably intravenously.

In other aspects, at least about three months after the administration, an imaging test (radiographic and/or MRI) is given that measures a reduction in bone and soft tissue joint damage as compared to baseline prior to the administration, and the amount of antagonist administered is effective in achieving a reduction in the joint damage. Preferably, the test measures a total modified Sharp score. Preferably, the antagonist is administered in a dose of about 0.2 to 4 grams, more preferably about 0.2 to 3.5 grams, more preferably about 0.4 to 2.5 grams, more preferably about 0.5 to 1.5 grams, and even more preferably about 0.7 to 1.1 gram. More preferably, such doses apply to antagonists that are antibodies or immunoadhesins.

Alternatively, the antagonist is anti-CD20 antibody administered at a dose of about 1000 mg×2 on days 1 and 15 intravenously at the start of the treatment. In another alternative preferred embodiment, the anti-CD20 antibody is administered as a single dose or as two infusions, with each dose at about 200 mg to 1.2 g, more preferably about 200 mg to 1.1 g, and still more preferably about 200 mg to 900 mg.

In a preferred aspect, the antagonist is administered at a frequency of one to four doses within a period of about one month. The antagonist is preferably administered in two to three doses. In addition, the antagonist is preferably administered within a period of about 2 to 3 weeks.

In another aspect, the B-cell antagonist is administered with no other medicament.

In an alternative aspect, the method further comprises administering an effective amount of one or more second medicaments with the B-cell antagonist. Preferably, the second medicament is more than one medicament. In another preferred aspect, the second medicament is an immunosuppressive agent, a DMARD, an integrin antagonist, a NSAID, a cytokine antagonist, a bisphosphonate, or a combination thereof. In one aspect, the second medicament is a DMARD, more preferably one selected from the group consisting of auranofin, chloroquine, D-penicillamine, injectable gold, oral gold, hydroxychloroquine, sulfasalazine, myocrisin, and MTX. In another aspect, the second medicament is a NSAID, more preferably one selected from the group consisting of: fenbufen, naprosyn, diclofenac, etodolac, indomethacin, aspirin, and ibuprofen. If the second medicament is an immunosuppressive agent, preferably it is selected from the group consisting of etanercept, infliximab, adalimumab, leflunomide, anakinra, azathioprine, and cyclophosphamide.

In another preferred aspect, the second medicament is selected from the group consisting of anti-alpha4, etanercept, infliximab, adalimumab, kinaret, efalizumab, osteoprotegerin (OPG), anti-receptor activator of NFκB ligand (anti-RANKL), anti-receptor activator of NFκB-Fc (RANK-Fc), pamidronate, alendronate, actonel, zolendronate, clodronate, MTX, azulfidine, hydroxychloroquine, doxycycline, leflunomide, sulfasalazine (SSZ), prednisolone, interleukin-1 receptor antagonist, prednisone, and methylprednisolone.

In still another embodiment, the second medicament is selected from the group consisting of infliximab, an infliximab/MTX combination, MTX, etanercept, a corticosteroid, cyclosporin A, azathioprine, auranofin, hydroxychloroquine (HCQ), a combination of prednisolone, MTX, and SSZ, a combination of MTX, SSZ, and HCQ, a combination of cyclophosphamide, azathioprine, and HCQ, and a combination of adalimumab with MTX, more preferably wherein the corticosteroid is prednisone, prednisolone, methylprednisolone, hydrocortisone, or dexamethasone. In another preferred aspect the second medicament is MTX, which is preferably administered perorally or parenterally.

In a still further embodiment, the arthritis is early or incipient RA.

In a preferred aspect, the treatment method further comprises re-treating the patient by administering an effective amount of the B-cell antagonist to the patient, wherein the re-treatment is commenced at least about 24 weeks (more preferably at about 24 weeks) after the first administration of the antagonist. In another preferred embodiment, a further re-treatment is commenced with an effective amount of the B-cell antagonist, more preferably at a time at least about 24 weeks (more preferably at about 24 weeks) after the second administration of the antagonist.

In a preferred embodiment the amount of the B-cell antagonist administered upon each administration thereof is effective to achieve a continued or maintained reduction in joint damage.

Another aspect of the invention involves a method of treating RA in a patient comprising first administering a B-cell antagonist to the patient to treat the RA, provided that a PTPN22 R620W SNP or SE or both SNP and SE are present in a genetic sample from the patient, and at least about 24 weeks after the first administration of the antagonist, re-treating the patient by administering an effective amount of the B-cell antagonist to the patient, wherein no clinical improvement is observed in the patient at the time of the testing after the first administration of the B-cell antagonist.

Preferably, the clinical improvement is determined by assessing the number of tender or swollen joints, conducting a global clinical assessment of the patient, assessing erythrocyte sedimentation rate, assessing the amount of C-reactive protein level, or using composite measures of disease activity (disease response), such as the DAS-28, ACR20, ACR50, or ACR70 scores.

In another embodiment the amount of the B-cell antagonist administered upon re-treatment in the above method is effective to achieve a continued or maintained reduction in joint damage as compared to the effect of a prior administration of the B-cell antagonist.

In another aspect, the invention provides a method for advertising a B-cell antagonist or a pharmaceutically acceptable composition thereof comprising promoting, to a target audience, the use of the antagonist or pharmaceutical composition thereof for treating a patient or patient population with RA from whom a genetic sample has been obtained showing the presence of a PTPN22 R620W SNP or SE, or both SNP and SE. Optionally, this method may include assessing seropositivity for at least one of the additional biomarkers anti-CCP and RF.

In another embodiment the invention provides an article of manufacture comprising, packaged together, a pharmaceutical composition comprising a B-cell antagonist and a pharmaceutically acceptable carrier and a label stating that the antagonist or pharmaceutical composition is indicated for treating patients with RA from whom a genetic sample has been obtained showing the presence of a PTPN22 R620W SNP or SE, or both SNP and SE. Optionally, this may include assessing seropositivity for one or both of the additional biomarkers anti-CCP and RF. In a preferred aspect, the article further comprises a container comprising a second medicament, wherein the antagonist is a first medicament, and also comprises instructions on the package insert for treating the patient with an effective amount of the second medicament, which is most preferably MTX.

In a still preferred aspect, the invention provides a method for manufacturing a B-cell antagonist or a pharmaceutical composition thereof comprising combining in a package the antagonist or pharmaceutical composition and a label stating that the antagonist or pharmaceutical composition is indicated for treating patients with RA from whom a genetic sample has been obtained showing the presence of a PTPN22 R620W SNP or SE, or both SNP and SE. Optionally, this method may include assessing seropositivity for one or both of the additional biomarkers anti-CCP and RF.

In yet another aspect, the invention supplies a method of providing a treatment option for patients with RA comprising packaging a B-cell antagonist in a vial with a package insert containing instructions to treat patients with RA from whom a genetic sample has been obtained showing the presence of a PTPN22 R620W SNP or SE, or both SNP and SE.

In a preferred embodiment, the samples from the patient, including genetic samples, are blood serum, blood plasma, or synovial tissue or fluid, most preferably blood. If anti-CCP and/or RF are also measured in a patient sample, the amount of such biomarkers may be determined by using, e.g., a reagent that specifically binds with the biomarker protein or a fragment thereof, such as, e.g., an antibody, a fragment of an antibody, or an antibody derivative.

The level of expression may be determined, for example, using a method selected from the group consisting of proteomics, flow cytometry, immunocytochemistry, immunohistochemistry, enzyme-linked immunosorbent assay (ELISA), multi-channel ELISA, and variations thereof. The expression level of a biomarker in the biological sample may also be determined by detecting the level of expression of a transcribed biomarker polynucleotide or fragment thereof encoded by a biomarker gene, which may be cDNA, mRNA or heterogeneous nuclear RNA (hnRNA). Detecting may include amplifying the transcribed biomarker polynucleotide, and may use the quantitative reverse transcriptase polymerase chain reaction (PCR). The expression level of a biomarker may be assessed by detecting the presence of the transcribed biomarker polynucleotide or a fragment thereof in a sample with a probe that anneals with the transcribed biomarker polynucleotide or fragment thereof under stringent hybridization conditions.

In another embodiment, the invention provides a method for predicting whether a subject with RA will respond to a B-cell antagonist, the method comprising determining whether a genetic sample from the subject shows the presence of a PTPN22 R620W SNP or shared epitope, or both SNP and shared epitope, wherein said presence indicates that the subject will respond to the antagonist.

In a still further embodiment, the invention provides a method of specifying a B-cell antagonist for use in a RA patient subpopulation, the method comprising providing instruction to administer the B-cell antagonist to a patient subpopulation characterized by the presence of a PTPN22 R620W SNP or shared epitope, or both SNP and shared epitope.

In a further embodiment, the invention provides a method for marketing a B-cell antagonist for use in a RA patient subpopulation, the method comprising informing a target audience about the use of the antagonist for treating the patient subpopulation characterized by the presence, in patients of such subpopulation, of a PTPN22 R620W SNP or shared epitope, or both SNP and shared epitope.

In a still further aspect, the invention supplies a method of assessing whether a sample from a patient with RA indicates responsiveness of the patient to treatment with a B-cell antagonist comprising:
a. detecting in the sample whether at least one biomarker that is PTPN22 R620W SNP or shared epitope is present;
b. implementing an algorithm to determine that the patient is responsive to said treatment; and
c. recording a result specific to the sample being tested.

Preferably, a computer or machine is used to record the result specific to the sample being tested.

In a still additional aspect, the invention supplies a system for analyzing susceptibility or responsiveness of a patient with RA to treatment with a B-cell antagonist comprising:
a. reagents to detect in a sample from the patient the biomarker PTPN22 R620W SNP or shared epitope, or both biomarkers SNP and shared epitope;
b. hardware to perform detection of the biomarkers; and
c. computational means to perform an algorithm to determine if the patient is susceptible or responsive to said treatment.

The reagents to detect the biomarker(s) may be, for example, antibodies, polynucleotides, and other molecules that bind to the SNP and/or shared epitope. The hardware is preferably a machine or computer to perform the detection step, and the computational means may be by, for example, computer or machine. An “algorithm” as used in the methods and systems herein is a specific set of instructions or a definite list of well-defined instructions for carrying out a procedure, typically proceeding through a well-defined series of successive states, and eventually terminating in an end-state, in this case, a binary answer of yes or no to the presence of the SNP and/or shared epitope.

### DETAILED DESCRIPTION OF THE PREFERRED EMBODIMENTS

### A. Definitions

A “B cell” is a lymphocyte that matures within the bone marrow, and includes a naïve B cell, memory B cell, or effector B cell (plasma cells). The B cell herein is a normal or non-malignant B cell.

A “B-cell malignancy” is a malignancy involving B cells. Examples include Hodgkin's disease, including lymphocyte predominant Hodgkin's disease (LPHD); non-Hodgkin's lymphoma (NHL); follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLD); Waldenstrom's macroglobulinemia (lymphoplasmacytic lymphoma); mucosa-associated lymphoid tissue (MALT) lymphoma; and marginal zone lymphoma/leukemia.

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition, ed. Barclay et al. (Academic Press, Harcourt Brace & Co., New York: 1997). Other B-cell surface markers include RP105, FcRH2, B-cell CR2, CCR6, P2×5, HLA-DOB, CXCR5, FCER2, BR3, BAFF, BLyS, Btig, NAG14, SLGC16270, FcRH1, IRTA2, ATWD578, FcRH3, IRTA1, FcRH6, BCMA, and 239287. The B-cell surface marker of particular interest is preferentially expressed on B cells compared to other non-B-cell tissues of a mammal and may be expressed on both precursor B cells and mature B cells. The preferred B-cell surface markers herein are CD20, CD22, CD23, CD40, BR3, BLyS, and BAFF.

The “CD20” antigen, or “CD20,” is an about 35-kDa, non-glycosylated phosphoprotein found on the surface of greater than 90% of B cells from peripheral blood or lymphoid organs. CD20 is present on both normal B cells and malignant B cells, but is not expressed on stem cells. Other names for CD20 in the literature include “B-lymphocyte-restricted antigen” and “Bp35.” The CD20 antigen is described in Clark et al., Proc. Natl. Acad. Sci. USA, 82:1766 (1985), for example. The preferred CD20 is human CD20.

The “CD22” antigen, or “CD22,” also known as BL-CAM or Lyb8, is a type 1 integral membrane glycoprotein with molecular weight of about 130 (reduced) to 140 kD (unreduced). It is expressed in both the cytoplasm and the cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells (CD22−). The CD22 antigen is described, e.g., in Wilson et al., J. Exp. Med., 173:137 (1991) and Wilson et al., J. Immunol., 150:5013 (1993). The preferred CD22 is human CD22.

A “B-cell antagonist” is a molecule that, upon binding to a B-cell surface marker or B-cell specific survival or proliferation factor, destroys or depletes B cells in a mammal and/or interferes with B-cell survival and/or one or more B-cell functions, e.g. by reducing or preventing a humoral response elicited by the B cell. The antagonist preferably is able to deplete B cells (i.e., reduce circulating B-cell levels) in a mammal treated therewith. Such depletion may be achieved via various mechanisms such as ADCC and/or CDC, inhibition of B-cell proliferation, and/or induction of B-cell death (e.g., via apoptosis). Antagonists can be screened by various methods known in the art for apoptosis and other measurements for the depletion, and retardation or stopping of proliferation and growth of B cells or survival of B cells.

For example, a method of screening can be employed as described in Sundberg et al., Cancer Research, 66, 1775-1782 (2006) wherein a compound was screened for inhibition of B-cell proliferation by targeting c-myc protein for rapid and specific degradation. See also Mackay et al., Annual Review of Immunology, 21: 231-264 (2003) regarding BAFF, APRIL, and a tutorial on B-cell survival and screening, and Thangarajh et al., Scandinavian J. Immunol., 65(1):92 (2007) on B-cell proliferation and APRIL. In addition, Sakurai et al., European J. Immunol., 37(1): 110 (2007) discloses that TACI attenuates antibody production co-stimulated by BAFF-R and CD40. Further, Acosta-Rodriguez et al., European J. Immunol., 37(4):990 (2007) discloses that BAFF and LPS cooperate to induce B cells to become susceptible to CD95/Fas-mediated cell death. Further screening methods can be found in Martin and Chan, “B Cell Immunobiology in Disease: Evolving Concepts from the Clinic,” Annual Review of Immunology, 24:467-496 (2006), Pillai et al., “Marginal Zone B Cells,” Annual Review of Immunology, 23:161-196 (2005), and Hardy and Hayakawa, “B Cell Development Pathways,” Annual Review of Immunology, 19:595-621 (2001). From these and other references the skilled artisan can screen for the appropriate antagonists. Microarrays can be used for this purpose (Hagmann, Science, 290:82-83 (2000)), as well as RNA interference (RNAi) (Ngo et al., Nature, 441:106-110 (2006)).

Antagonists included within the scope of the present invention include antibodies, synthetic or native-sequence peptides, immunoadhesins, and small-molecule antagonists that bind to a B-cell surface marker or a B-cell specific survival or proliferation factor, optionally conjugated with or fused to another molecule. The preferred antagonist comprises an antibody or immunoadhesin. It includes BLyS antagonists such as immunoadhesins, and is preferably anti-CD23 (e.g., lumiliximab), anti-CD20, anti-CD22, or anti-BR3 antibodies, APRIL antagonists, and/or BlyS immunoadhesins. The BlyS immunoadhesin preferably is selected from the group consisting of BR3 immunoadhesin comprising the extracellular domain of BR3, TACI immunoadhesin comprising the extracellular domain of TACI, and BCMA immunoadhesin comprising the extracellular domain of BCMA. The most preferred BR3 immunoadhesin is hBR3-Fc of SEQ ID NO:2 of WO 2005/00351 and US 2005/0095243. See also US 2005/0163775 and WO 2006/068867. Another preferred BLyS antagonist is an anti-BLyS antibody, more preferably wherein the anti-BLyS antibody binds BLyS within a region of BLyS comprising residues 162-275, or an anti-BR3 antibody, more preferably wherein the anti-BR3 antibody binds BR3 in a region comprising residues 23-38 of human BR3. Especially preferred immunoadhesins herein are TACI-Ig, or atacicept, and BR3-Ig. A preferred set of antagonists are to CD20, CD22, BAFF, or APRIL. The antagonist may be, in one aspect, an antibody or TACI-Ig.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

An “isolated” antibody is one that has been identified and separated and/or recovered from a component of its natural environment. Contaminant components of its natural environment are materials that would interfere with research, diagnostic, or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

An “antibody antagonist” herein is an antibody that, upon binding to a B-cell surface marker on B cells, destroys or depletes B cells in a mammal and/or interferes with one or more B-cell functions, e.g., by reducing or preventing a humoral response elicited by the B cell. The antibody antagonist preferably is able to deplete B cells (i.e., reduce circulating B-cell levels) in a mammal treated therewith. Such depletion may be achieved via various mechanisms such as ADCC and/or CDC, inhibition of B-cell proliferation, and/or induction of B-cell death (e.g., via apoptosis).

An “antibody that binds to a B-cell surface marker” or “antibody to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody preferably is able to deplete B cells (i.e. reduce circulating B-cell levels) in a mammal treated therewith. Such depletion may be achieved via various mechanisms such as ADCC and/or CDC, inhibition of B-cell proliferation and/or induction of B-cell death (e.g. via apoptosis). The antibody that binds to a B-cell surface marker may be designated as follows: an antibody that binds to CD20 or CD22 is an “anti-CD20 antibody” or “anti-CD22 antibody,” respectively. In a preferred embodiment, the antibody is an anti-CD20, anti-CD22, anti-CD 23, anti-CD40, or anti-BR3 antibody. A more preferred antibody is an anti-CD20, anti-CD22, or anti-BR3 antibody. A particularly preferred embodiment is an anti-CD20 or anti-CD22 antibody, and most preferably the antibody is an anti-CD20 antibody.

Examples of anti-CD20 antibodies include: “C2B8,” which is now called “rituximab” (“RITUXAN®/MABTHERA®”) (U.S. Pat. No. 5,736,137); the yttrium-[90]-labelled 2B8 murine antibody designated “Y2B8” or “Ibritumomab Tiuxetan” (ZEVALIN®) commercially available from Biogen Idec, Inc. (e.g., U.S. Pat. No. 5,736,137; 2B8 deposited with the American Type Culture Collection (ATCC) as No. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, e.g., U.S. Pat. No. 5,595,721); murine monoclonal antibody “1F5” (e.g., Press et al., Blood, 69(2):584-591 (1987) and variants thereof including “framework patched” or humanized 1F5 (e.g., WO 2003/002607, Leung; ATCC deposit HB-96450); murine 2H7 and chimeric 2H7 antibody (e.g., U.S. Pat. No. 5,677,180); a 2H7 antibody (e.g., WO 2004/056312 (Lowman et al.) and as set forth below); HUMAX-CD20™ (ofatumumab) fully human, high-affinity antibody targeted at the CD20 molecule in the cell membrane of B-cells (Genmab, Denmark; see, for example, Glennie and van de Winkel, Drug Discovery Today, 8:503-510 (2003) and Cragg et al., Blood, 101: 1045-1052 (2003)); the human monoclonal antibodies set forth in WO 2004/035607 and WO 2005/103081 (Teeling et al., GenMab/Medarex); the antibodies having complex N-glycoside-linked sugar chains bound to the Fc region described in US 2004/0093621 (Shitara et al.); a chimerized or humanized monoclonal antibody having a high binding affinity to an extracellular epitope of a CD20 antigen described in WO 2006/106959 (Numazaki et al., Biomedics Inc.); monoclonal antibodies and antigen-binding fragments binding to CD20 (e.g., WO 2005/000901, Tedder et al.) such as HB20-3, HB20-4, HB20-25, and MB20-11; single-chain proteins binding to CD20 including, but not limited to, TRU-015™ (e.g., US 2005/0186216 (Ledbetter and Hayden-Ledbetter); US 2005/0202534 (Hayden-Ledbetter and Ledbetter); US 2005/0202028 (Hayden-Ledbetter and Ledbetter); US 2005/136049 (Ledbetter et al.); and US 2005/0202023 (Hayden-Ledbetter and Ledbetter)-Trubion Pharm Inc.); CD20-binding molecules such as the AME series of antibodies, e.g., AME-33™ and AME-133™ antibodies as set forth, for example, in WO 2004/103404; US 2005/0025764; and US 2006/0251652 (Watkins et al., Applied Molecular Evolution, Inc.) and the anti-CD20 antibodies with Fc mutations as set forth, for example, in WO 2005/070963 (Allan et al., Applied Molecular Evolution, Inc.); CD20-binding molecules such as those described in WO 2005/016969 and US 2005/0069545 (Carr et al.); bispecific antibodies as set forth, for example, in WO 2005/014618 (Chang et al.); humanized LL2 monoclonal antibodies and other anti-CD20 antibodies as described, for example, in U.S. Pat. No. 7,151,164 (Hansen et al., US 2005/0106108 (Leung and Hansen; Immunomedics)); fully human antibodies against CD20 as described, e.g., in WO 2006/130458; Gazit et al., Amgen/AstraZeneca); antibodies against CD20 as described, for example, in WO 2006/126069 (Morawala, Avestha Gengraine Technologies Pvt Ltd.); chimeric or humanized B-Ly1 antibodies to CD20 (e.g., GA-101) as described, for example, in WO 2005/044859; US 2005/0123546; US 2004/0072290; and US 2003/0175884 (Umana et al.; GlycArt Biotechnology AG); A20 antibody or variants thereof such as chimeric or humanized A20 antibody (cA20, hA20, respectively) and IMMUN-106 (e.g., US 2003/0219433, Immunomedics); and monoclonal antibodies L27, G28-2, 93-1B3, B-C1 or NU-B2 available from the International Leukocyte Typing Workshop (e.g., Valentine et al., In: Leukocyte Typing III (McMichael, Ed., p. 440, Oxford University Press (1987)). The preferred anti-CD20 antibodies herein are chimeric, humanized, or human anti-CD20 antibodies, more preferably rituximab, a 2H7 antibody, chimeric or humanized A20 antibody (Immunomedics), and HUMAX-CD20 human anti-CD 20 antibody (Genmab).

Examples of anti-CD22 antibodies include the ones described in EP 1,476,120 (Tedder and Tuscano), EP 1,485,130 (Tedder), and EP 1,504,035 (Popplewell et al.), as well as those described in US 2004/0258682 (Leung et al.), U.S. Pat. No. 5,484,892 (Dana-Farber), U.S. Pat. No. 6,183,744 (Immunomedics, epratuzumab), and U.S. Pat. No. 7,074,403 (Goldenberg and Hansen).

Preferred specific examples of antibodies to B-cell surface markers include rituximab, a 2H7 antibody and variants thereof as defined herein, 2F2 (HUMAX-CD20™) (ofatumumab) human anti-CD20 antibody (an IgG1κ human MAb that binds to a different CD20 epitope than rituximab), humanized A20 antibody veltuzumab (IMMUN-106™ or hA20), a humanized engineered antibody with complementarity-determining regions (CDRs) of murine origin and with 90% of the human framework regions identical to epratuzumab (a humanized anti-CD22 IgG1 antibody); a small, modular immunopharmaceutical (SMIP) (herein called immunopharmaceutical) having SEQ ID NO:16 (also known as TRU-015), a CD20-binding molecule that is an antibody comprising SEQ ID NOS:17 and 18 (Lilly AME 33) or SEQ ID NOS:19 and 20 (Lilly AME 133) or SEQ ID NO:21 (Lilly AME 133v, otherwise known as LY2469298, which binds with an increased affinity to the FcγRIIIa (CD16)), a humanized type II anti-CD20 antibody of the isotype IgG1 with a glycoengineered Fc portion (bisected afucosylated carbohydrates in the Fc region) and a modified elbow hinge, known as GA101 (see SEQ ID NOS:22-23 below), anti-BAFF antibody, anti-APRIL antibodies, anti-BR3 antibody, anti-BAFF receptor antibody, anti-BLyS antibody, anti-CD23 antibody such as lumiliximab, anti-CD37 antibody and antagonists including the small modular immunopharmaceutical drug TRU016™, anti-CD 40 antibody, and anti-CD22 antibody such as epratuzumab, ABIOGEN™ anti-CD22 antibody, and IMPHERON™ anti-B cell antibody. Preferred examples of immunoadhesins herein include BR3-Ig, BR3-Fc, and APRIL immunoadhesins such as TACI-Ig, anti-BAFF peptibody, BCMA-Ig, and BAFF-R-Ig (US 2006/0263349).

The TRU-015 polypeptide sequence is:

(SEQ ID NO: 16)
Met Asp Phe Gln Val Gln Ile Phe Ser Phe Leu Leu Ile Ser Ala Ser Val Ile Met Ser Arg Gly Gln
Ile Val Leu Ser Gln Ser Pro Ala Ile Leu Ser Ala Ser Pro Gly Glu Lys Val Thr Met Thr Cys Arg
Ala Ser Ser Ser Val Ser Tyr Met His Trp Tyr Gln Gln Lys Pro Gly Ser Ser Pro Lys Pro Trp Ile
Tyr Ala Pro Ser Asn Leu Ala Ser Gly Val Pro Ala Arg Phe Ser Gly Ser Gly Ser Gly Thr Ser Tyr
Ser Leu Thr Ile Ser Arg Val Glu Ala Glu Asp Ala Ala Thr Tyr Tyr Cys Gln Gln Trp Ser Phe
Asn Pro Pro Thr Phe Gly Ala Gly Thr Lys Leu Glu Leu Lys Asp Gly Gly Gly Ser Gly Gly Gly
Gly Ser Gly Gly Gly Gly Ser Ser Gln Ala Tyr Leu Gln Gln Ser Gly Ala Glu Ser Val Arg Pro
Gly Ala Ser Val Lys Met Ser Cys Lys Ala Ser Gly Tyr Thr Phe Thr Ser Tyr Asn Met His Trp
Val Lys Gln Thr Pro Arg Gln Gly Leu Glu Trp Ile Gly Ala Ile Tyr Pro Gly Asn Gly Asp Thr
Ser Tyr Asn Gln Lys Phe Lys Gly Lys Ala Thr Leu Thr Val Asp Lys Ser Ser Ser Thr Ala Tyr
Met Gln Leu Ser Ser Leu Thr Ser Glu Asp Ser Ala Val Tyr Phe Cys Ala Arg Val Val Tyr Tyr
Ser Asn Ser Tyr Trp Tyr Phe Asp Val Trp Gly Thr Gly Thr Thr Val Thr Val Ser Asp Gln Glu
Pro Lys Ser Cys Asp Lys Thr His Thr Ser Pro Pro Cys Ser Ala Pro Glu Leu Leu Gly Gly Pro Ser
Val Phe Leu Phe Pro Pro Lys Pro Lys Asp Thr Leu Met Ile Ser Arg Thr Pro Glu Val Thr Cys Val
Val Val Asp Val Ser His Glu Asp Pro Glu Val Lys Phe Asn Trp Tyr Val Asp Gly Val Glu Val
His Asn Ala Lys Thr Lys Pro Arg Glu Glu Gln Tyr Asn Ser Thr Tyr Arg Val Val Ser Val Leu
Thr Val Leu His Gln Asp Trp Leu Asn Gly Lys Glu Tyr Lys Cys Lys Val Ser Asn Lys Ala Leu
Pro Ala Pro Ile Glu Lys Thr Ile Ser Lys Ala Lys Gly Gln Pro Arg Glu Pro Gln Val Tyr Thr Leu
Pro Pro Ser Arg Asp Glu Leu Thr Lys Asn Gln Val Ser Leu Thr Cys Leu Val Lys Gly Phe Tyr
Pro Ser Asp Ile Ala Val Glu Trp Glu Ser Asn Gly Gln Pro Glu Asn Asn Tyr Lys Thr Thr Pro Pro
Val Leu Asp Ser Asp Gly Ser Phe Phe Leu Tyr Ser Lys Leu Thr Val Asp Lys Ser Arg Trp Gln
Gln Gly Asn Val Phe Ser Cys Ser Val Met His Glu Ala Leu His Asn His Tyr Thr Gln Lys Ser
Leu Ser Leu Ser Pro Gly Lys

See also US 2007/0059306.

The polypeptide representing the light-chain variable region of the AME 33 antibody has the following sequence:

(SEQ ID NO: 17)
Glu Ile Val Leu Thr Gln Ser Pro Gly Thr Leu Ser Leu Ser Pro Gly Glu Arg Ala
Thr Leu Ser Cys Arg Ala Ser Ser Ser Val Pro Tyr Ile His Trp Tyr Gln Gln Lys
Pro Gly Gln Ala Pro Arg Leu Leu Ile Tyr Ala Thr Ser Ala Leu Ala Ser Gly Ile
Pro Asp Arg Phe Ser Gly Ser Gly Ser Gly Thr Asp Phe Thr Leu Thr Ile Ser Arg
Leu Glu Pro Glu Asp Phe Ala Val Tyr Tyr Cys Gln Gln Trp Leu Ser Asn Pro Pro
Thr Phe Gly Gln Gly Thr Lys Leu Glu Ile Lys

The polypeptide representing the heavy-chain variable region of the AME 33 antibody has the following sequence:

(SEQ ID NO: 18)
Glu Val Gln Leu Val Gln Ser Gly Ala Glu Val Lys Lys Pro Gly Glu Ser Leu Lys
Ile Ser Cys Lys Gly Ser Gly Arg Thr Phe Thr Ser Tyr Asn Met His Trp Val Arg
Gln Met Pro Gly Lys Gly Leu Glu Trp Met Gly Ala Ile Tyr Pro Leu Thr Gly Asp
Thr Ser Tyr Asn Gln Lys Ser Lys Leu Gln Val Thr Ile Ser Ala Asp Lys Ser Ile Ser
Thr Ala Tyr Leu Gln Trp Ser Ser Leu Lys Ala Ser Asp Thr Ala Met Tyr Tyr Cys
Ala Arg Ser Thr Tyr Val Gly Gly Asp Trp Gln Phe Asp Val Trp Gly Lys Gly Thr
Thr Val Thr Val Ser Ser

See also FIGS. 2-3 as well as SEQ ID NOS:59-62 of US 2005/0025764 and US 2006/0251652, for light- and heavy-chain variable region nucleotide and amino acid AME 33 sequences.

The polypeptide representing the light-chain variable region of the AME 133 antibody has the following sequence:

(SEQ ID NO:19)
Glu Ile Val Leu Thr Gln Ser Pro Gly Thr Leu Ser Leu Ser Pro Gly Glu Arg Ala
Thr Leu Ser Cys Arg Ala Ser Ser Ser Val Pro Tyr Ile His Trp Tyr Gln Gln Lys
Pro Gly Gln Ala Pro Arg Leu Leu Ile Tyr Ala Thr Ser Ala Leu Ala Ser Gly Ile
Pro Asp Arg Phe Ser Gly Ser Gly Ser Gly Thr Asp Phe Thr Leu Thr Ile Ser Arg
Leu Glu Pro Glu Asp Phe Ala Val Tyr Tyr Cys Gln Gln Trp Leu Ser Asn Pro Pro
Thr Phe Gly Gln Gly Thr Lys Leu Glu Ile Lys Arg Thr Val Ala Ala Pro Ser Val
Phe Ile Phe Pro Pro Ser Asp Glu Gln Leu Lys Ser Gly Thr Ala Ser Val Val Cys
Leu Leu Asn Asn Phe Tyr Pro Arg Glu Ala Lys Val Gln Trp Lys Val Asp Asn
Ala Leu Gln Ser Gly Asn Ser Gln Glu Ser Val Thr Glu Gln Asp Ser Lys Asp Ser
Thr Tyr Ser Leu Ser Ser Thr Leu Thr Leu Ser Lys Ala Asp Tyr Glu Lys His Lys
Val Tyr Ala Cys Glu Val Thr His Gln Gly Leu Ser Ser Pro Val Thr Lys Ser Phe
Asn Arg Gly Glu Cys

The polypeptide representing the heavy-chain variable region of the AME 133 antibody has the following sequence:

(SEQ ID NO: 20)
Glu Val Gln Leu Val Gln Ser Gly Ala Glu Val Lys Lys Pro Gly Glu Ser Leu Lys
Ile Ser Cys Lys Gly Ser Gly Arg Thr Phe Thr Ser Tyr Asn Met His Trp Val Arg
Gln Met Pro Gly Lys Gly Leu Glu Trp Met Gly Ala Ile Tyr Pro Leu Thr Gly Asp
Thr Ser Tyr Asn Gln Lys Ser Lys Leu Gln Val Thr Ile Ser Ala Asp Lys Ser Ile Ser
Thr Ala Tyr Leu Gln Trp Ser Ser Leu Lys Ala Ser Asp Thr Ala Met Tyr Tyr Cys
Ala Arg Ser Thr Tyr Val Gly Gly Asp Trp Gln Phe Asp Val Trp Gly Lys Gly Thr
Thr Val Thr Val Ser Ser

See also US 2005/0136044.

The polypeptide representing AME 133v, a fusion protein prepared from the AME 133 Fab region fused to modified BChE variant L530, has the following sequence:

(SEQ ID NO: 21)
Glu Val Gln Leu Val Gln Ser Gly Ala Glu Val Lys Lys Pro Gly Gln Ser Leu Lys
Ile Ser Cys Lys Gly Ser Gly Arg Thr Phe Thr Ser Tyr Asn Met His Trp Val Arg
Gln Met Pro Gly Lys Gly Leu Glu Trp Met Gly Ala Ile Tyr Pro Leu Thr Gly Asp
Thr Ser Tyr Asn Gln Lys Ser Lys Leu Gln Val Thr Ile Ser Ala Asp Lys Ser Ile Ser
Thr Ala Tyr Leu Gln Trp Ser Ser Leu Lys Ala Ser Asp Thr Ala Met Tyr Tyr Cys
Ala Arg Ser Thr Tyr Val Gly Gly Asp Trp Gln Phe Asp Val Trp Gly Lys Gly Thr
Thr Val Thr Val Ser Ser Ala Ser Thr Lys Gly Pro Ser Val Phe Pro Leu Ala Pro
Ser Ser Lys Ser Thr Ser Gly Gly Thr Ala Ala Leu Gly Cys Leu Val Lys Asp Tyr
Phe Pro Glu Pro Val Thr Val Ser Trp Asn Ser Gly Ala Leu Thr Ser Gly Val His
Thr Phe Pro Ala Val Leu Gln Ser Ser Gly Leu Tyr Ser Leu Ser Ser Val Val Thr
Val Pro Ser Ser Ser Leu Gly Thr Gln Thr Tyr Ile Cys Asn Val Asn His Lys Pro
Ser Asn Thr Lys Val Asp Lys Lys Ala Gln Pro Lys Ser Cys Asp Lys Thr His Thr
Cys Pro Pro Cys Pro Lys Leu Glu Asp Asp Ile Ile Ile Ala Thr Lys Asn Gly Lys
Val Arg Gly Met Asn Leu Thr Val Phe Gly Gly Thr Val Thr Ala Phe Leu Gly Ile
Pro Tyr Ala Gln Pro Pro Leu Gly Arg Leu Arg Phe Lys Lys Pro Gln Ser Leu Thr
Lys Trp Ser Asp Ile Trp Asn Ala Thr Lys Tyr Ala Asn Ser Cys Cys Gln Asn Ile
Asp Gln Ser Phe Pro Gly Phe Phe Gly Ser Glu Met Trp Asn Pro Asn Thr Asp Leu
Ser Glu Asp Cys Leu Tyr Leu Asn Val Trp Ile Pro Ala Pro Lys Pro Lys Asn Ala
Thr Val Leu Ile Trp Ile Tyr Gly Gly Gly Phe Gln Thr Gly Thr Ser Ser Leu His
Val Tyr Asp Gly Lys Phe Leu Ala Arg Val Glu Arg Val Ile Val Val Ser Met Asn
Tyr Arg Val Gly Ala Leu Gly Phe Leu Ala Leu Pro Gly Asn Pro Glu Ala Pro Gly
Asn Met Gly Leu Phe Asp Gln Gln Leu Ala Leu Gln Trp Val Gln Lys Asn Ile Ala
Ala Phe Gly Gly Asn Pro Lys Ser Val Thr Leu Phe Gly Glu Ser Ala Gly Ala Ala
Ser Val Ser Leu His Leu Leu Ser Pro Gly Ser His Ser Leu Phe Thr Arg Ala Ile
Leu Gln Ser Gly Ser Ala Asn Ala Pro Trp Ala Val Thr Ser Leu Tyr Glu Ala Arg
Asn Arg Thr Leu Asn Leu Ala Lys Leu Thr Gly Cys Ser Arg Glu Asn Glu Thr
Glu Ile Ile Lys Cys Leu Arg Asn Lys Asp Pro Gln Glu Ile Leu Leu Asn Glu Ala
Phe Val Val Pro Tyr Gly Thr Asn Leu Ser Val Asn Phe Gly Pro Thr Val Asp Gly
Asp Phe Leu Thr Asp Met Pro Asp Ile Leu Leu Glu Leu Gly Gln Phe Lys Lys Thr
Gln Ile Leu Val Gly Val Asn Lys Asp Glu Gly Thr Ala Phe Leu Ala Tyr Gly Ala
Pro Gly Phe Ser Lys Asp Asn Asn Ser Ile Ile Thr Arg Lys Glu Phe Gln Glu Gly
Leu Lys Ile Phe Phe Pro Gly Val Ser Glu Phe Gly Lys Glu Ser Ile Leu Phe His
Tyr Thr Asp Trp Val Asp Asp Gln Arg Pro Glu Asn Tyr Arg Glu Ala Leu Gly
Asp Val Val Gly Asp Tyr Asn Phe Ile Cys Pro Ala Leu Glu Phe Thr Lys Lys Phe
Ser Glu Trp Gly Asn Asn Ala Phe Phe Tyr Tyr Phe Glu His Arg Ser Ser Lys Leu
Pro Trp Pro Glu Trp Met Gly Val Met His Gly Tyr Glu Ile Glu Phe Val Phe Gly
Leu Pro Leu Glu Arg Arg Asp Asn Tyr Thr Lys Ala Glu Glu Ile Leu Ser Arg Ser
Ile Val Lys Arg Trp Ala Asn Phe Ala Lys Tyr Gly Asn Pro Asn Glu Thr Gln Asn
Asn Ser Thr Ser Trp Pro Val Phe Lys Ser Thr Glu Gln Lys Tyr Leu Thr Leu Asn
Thr Glu Ser Thr Arg Ile Met Thr Lys Leu Arg Ala Gln Gln Cys Arg Phe Trp Thr
Ser Phe Phe Pro Lys Val

See also SEQ ID NO:202 and FIG. 19 from US 2005/0136044.

The polypeptide representing the light-chain variable region of the humanized type II anti-CD20 IgG1 antibody (GA101) has the following sequence:

(SEQ ID NO: 22)
Asp Ile Val Met Thr Gln Thr Pro Leu Ser Leu Pro Val Thr Pro Gly Glu Pro Ala Ser Ile Ser
Cys Arg Ser Ser Lys Ser Leu Leu His Ser Asn Gly Ile Thr Tyr Leu Tyr Trp Tyr Leu Gln Lys
Pro Gly Gln Ser Pro Gln Leu Leu Ile Tyr Gln Met Ser Asn Leu Val Ser Gly Val Pro Asp Arg
Phe Ser Gly Ser Gly Ser Gly Thr Asp Phe Thr Leu Lys Ile Ser Arg Val Glu Ala Glu Asp Val
Gly Val Tyr Tyr Cys Ala Gln Asn Leu Glu Leu Pro Tyr Thr Phe Gly Gly Gly Thr Lys Val
Gln Ile Lys Arg Thr Val

The polypeptide representing the heavy-chain variable region of the humanized type II anti-CD20 IgG1 antibody (GA101) has the following sequence:

(SEQ ID NO: 23)
Gln Val Gln Leu Val Gln Ser Gly Ala Glu Val Lys Lys Pro Gly Ser Ser Val Lys Val Ser Cys
Lys Ala Ser Gly Tyr Ala Phe Ser Tyr Ser Trp Met Asn Trp Val Arg Gln Ala Pro Gly Gln
Gly Leu Glu Trp Met Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr Met Glu Leu Ser Ser Leu
Arg Ser Glu Asp Thr Ala Val Tyr Tyr Cys Ala Arg Asn Val Phe Asp Gly Tyr Trp Leu Val
Tyr Trp Gly Gln Gly Thr Leu Val Thr Val Ser Ser

See also US 2005/0123546 regarding BHH2-KV1-GE (GA101), which was humanized by grafting CDR sequences from murine B-ly1 on framework regions with fully human IgG1-kappa germline sequences. FIG. 7 of US 2005/0123546 lists a selection of predicted CDR regions of B-ly1. The sequence for the BHH2 component of GA101 (the heavy-chain variable region) is presented in Tables 2 and 3 as SEQ ID NOS:31 (nucleotide) and 32 (amino acid). The KV1 component (the light-chain variable region) is presented in Tables 2 and 3 as SEQ ID NOS:75 (nucleotide) and 76 (amino acid). The apparent variable heavy-chain and light-chain signal sequences are also set forth in these Tables as SEQ ID NOS:73 (variable heavy-chain, nucleotide), 74 (variable heavy-chain, amino acid), 77 (variable light-chain, nucleotide), and 76 (variable light-chain, amino acid).

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in ADCC.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W. B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields a F(ab′)₂ fragment that has two antigen-combining sites and is still capable of cross-linking antigen.

“Fv” is the minimum antibody fragment that contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody-hinge region. Fab′-SH is the designation herein for Fab′ in which the cysteine residue(s) of the constant domains bear(s) a free thiol group. F(ab′)₂ antibody fragments originally were produced as pairs of Fab′ fragments that have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, see, e.g., Pluckthün, in The Pharmacology of Mono-clonal Antibodies, vol. 113, Rosenburg and Moore eds. (Springer-Verlag, New York: 1994), pp 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med., 9:129-134 (2003); and Hollinger et al., Proc. Natl. Acad. Sci. USA, 90:6444-6448 (1993). Triabodies and tetrabodies are also described in Hudson et al., Nat. Med., 9:129-134 (2003).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-497 (1975); Hongo et al., Hybridoma, 14 (3):253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol., 222:581-597 (1992); Sidhu et al., J. Mol. Biol., 338(2):299-310 (2004); Lee et al., J. Mol. Biol., 340(5):1073-1093 (2004); Fellouse, Proc. Natl. Acad. Sci. USA, 101(34):12467-12472 (2004); and Lee et al., J. Immunol. Methods, 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immunol., 7:33 (1993); U.S. Pat. No. 5,545,807; U.S. Pat. No. 5,545,806; U.S. Pat. No. 5,569,825; U.S. Pat. No. 5,625,126; U.S. Pat. No. 5,633,425; and U.S. Pat. No. 5,661,016; Marks et al., Bio/Technology, 10:779-783 (1992); Lonberg et al., Nature, 368:856-859 (1994); Morrison, Nature, 368:812-813 (1994); Fishwild et al., Nature Biotechnol., 14:845-851 (1996); Neuberger, Nature Biotechnol., 14:826 (1996); and Lonberg and Huszar, Intern. Rev. Immunol., 13:65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., Proc. Natl. Acad. Sci. USA, 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all, or substantially all, of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature, 321:522-525 (1986); Riechmann et al., Nature, 332:323-329 (1988); and Presta, Curr. Op. Struct. Biol., 2:593-596 (1992). See also, for example, Vaswani and Hamilton, Ann. Allergy, Asthma & Immunol., 1: 105-115 (1998); Harris, Biochem. Soc. Transactions, 23:1035-1038 (1995); Hurle and Gross, Curr. Op. Biotech., 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985) and Boerner et al., J. Immunol., 147(1):86-95 (1991). See also van Dijk and van de Winkel, Curr. Opin. Pharmacol., 5:368-374 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. No. 6,075,181 and U.S. Pat. No. 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein, refers to the regions of an antibody-variable domain that are hypervariable in sequence and/or form structurally defined loops. Generally, antibodies comprise six HVRs; three in the VH(H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity, 13:37-45 (2000); Johnson and Wu in Methods in Molecular Biology, 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature, 363:446-448 (1993) and Sheriff et al., Nature Struct. Biol., 3:733-736 (1996).

A number of HVR delineations are in use and encompassed herein. The HVRs that are Kabat CDRs are based on sequence variability and are the most commonly used (Kabat et al., supra). Chothia refers instead to the location of the structural loops. Chothia and Lesk, J. Mol. Biol., 196:901-917 (1987). The AbM HVRs represent a compromise between the Kabat CDRs and Chothia structural loops, and are used by Oxford Molecular's AbM antibody-modeling software. The “contact” HVRs are based on an an-alysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat-numbered sequence.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 10:779-783 (1992) describes affinity maturation by VH- and VL-domain shuffling. Random mutagenesis of HVR and/or framework residues is described by, for example, Barbas et al., Proc Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7):3310-9 (1995); and Hawkins et al., J. Mol. Biol., 226:889-896 (1992).

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g., of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

Antibody “effector functions” refer to those biological activities attributable to the Fc region (a native-sequence Fc region or amino-acid-sequence-variant Fc region) of an antibody, and vary with the antibody isotype. Examples of antibody effector functions include: C1q binding and CDC; Fc-receptor binding; ADCC; phagocytosis; down-regulation of cell-surface receptors (e.g., B-cell receptor); and B-cell activation.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

A “functional Fc region” possesses an “effector function” of a native-sequence Fc region. Exemplary “effector functions” include C1q binding; CDC; Fc-receptor binding; ADCC; phagocytosis; down-regulation of cell-surface receptors (e.g., B-cell receptor; BCR), etc. Such effector functions generally require the Fc region to be combined with a binding domain (e.g., an antibody-variable domain) and can be assessed using various assays as disclosed, for example, in definitions herein.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, a composition comprising an antibody having an Fc region according to this invention can comprise an antibody with K447, with all K447 removed, or a mixture of antibodies with and without the K447 residue.

“Fc receptor” or “FcR” describes a receptor that binds to the Fc region of an antibody. In some embodiments, an FcR is a native-human FcR. In some embodiments, an FcR is one that binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see, e.g., Daëiron, Annu. Rev. Immunol., 15:203-234 (1997)). FcRs are reviewed, for example, in Ravetch and Kinet, Annu. Rev. Immunol, 9:457-492 (1991); Capel et al., Immunomethods, 4:25-34 (1994); and de Haas et al., J. Lab. Clin. Med., 126:330-341 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol., 117:587 (1976) and Kim et al., Eur. J. Immunol., 24:2429-2434 (1994)) and regulation of homeostasis of immunoglobulins. Methods of measuring binding to FcRn are known (see, e.g., Ghetie and Ward, Immunology Today, 18 (12):592-598 (1997); Ghetie et al., Nature Biotechnology, 15 (7):637-640 (1997); Hinton et al., J. Biol. Chem., 279(8):6213-6216 (2004); WO 2004/92219 (Hinton et al.)).

Binding to human FcRn in vivo and serum half-life of human FcRn high-affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See, also, for example, Shields et al., J. Biol. Chem., 9(2):6591-6604 (2001).

“Human effector cells” are leukocytes that express one or more FcRs and perform effector functions. In certain embodiments, the cells express at least FcγRIII and perform ADCC effector function(s). Examples of human leukocytes that mediate ADCC include peripheral blood mononuclear cells (PBMC), natural-killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enables these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol., 9:457-492 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA), 95:652-656 (1998).

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods, 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 and WO 1999/51642. See, also, e.g., Idusogie et al., J. Immunol., 164:4178-4184 (2000).

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol., 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc#269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res., 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd or Kd value is measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl per minute, to achieve approximately ten response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol., 293:865-881 (1999). If the on-rate exceeds 10⁶ M⁻¹s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N.J.).

The term “substantially similar” or “substantially the same,” as used herein, denotes a sufficiently high degree of similarity between two numeric values (e.g., one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “rituximab” or “RITUXAN®” herein refers to the genetically engineered chimeric murine/human monoclonal antibody directed against the CD20 antigen and designated “C2B8” in U.S. Pat. No. 5,736,137, including fragments thereof that retain the ability to bind CD20.

Purely for the purposes herein and unless indicated otherwise, “2H7” or “2H7 antibody” refers to a humanized anti-CD20 antibody with the sequences provided immediately below and/or described in US 2006/0034835 and WO 2004/056312 (both Lowman et al.); US 2006/0188495 (Barron et al.); and US 2006/0246004 (Adams et al.). Briefly, humanization of the murine anti-human CD20 antibody, 2H7 (also referred to herein as m2H7, m for murine), was carried out in a series of site-directed mutagenesis steps. The murine 2H7 antibody variable region sequences and the chimeric 2H7 with the mouse V and human C have been described, e.g., in U.S. Pat. No. 5,846,818 and U.S. Pat. No. 6,204,023. The CDR residues of 2H7 were identified by comparing the amino acid sequence of the murine 2H7 variable domains (disclosed in U.S. Pat. No. 5,846,818) with the sequences of known antibodies (Kabat et al., supra). The CDR regions for the light and heavy chains were defined based on sequence hypervariability (Kabat et al., supra). Using synthetic oligonucleotides, site-directed mutagenesis (Kunkel, Proc. Natl. Acad. Sci. USA, 82:488-492 (1985)) was used to introduce all six of the murine 2H₇CDR regions into a complete human Fab framework corresponding to a consensus sequence V_{κ}I, V_{H}III (V_{L} kappa subgroup I, V_{H} subgroup III) contained on plasmid pVX4 (see FIG. 2 in WO 2004/056312). Further modifications of the V regions (CDR and/or FR) were made in the phagemid pVX4 by site-directed mutagenesis. Plasmids for expression of full-length IgG's were constructed by subcloning the V_{L} and V_{H} domains of chimeric 2H7Fab as well as humanized Fab versions 2 to 6 into previously described pRK vectors for mammalian cell expression (Gorman et al., DNA Prot. Eng. Tech., 2:3-10 (1990)).

The following 2H7 antibodies are included within the definition herein:

(1) A humanized antibody comprising the VL sequence:

(SEQ ID NO: 1)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKR;

and the VH sequence:

(SEQ ID NO: 2)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSS.

(2) A humanized antibody comprising the VL sequence:

(SEQ ID NO: 3)

DIQMTQSPSSLSASVGDRVTITCRASSSVSYLHWYQQKPGKAPKPLIYAP
SNLASGVPSRFSGSGSGTDFTLTISSLQPEDFATYYCQQWAENPPTFGQG
TKVEIKR;

and the VH sequence:

(SEQ ID NO: 4)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSS.

(3) A humanized antibody comprising the VL sequence:

(SEQ ID NO: 3)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR;

and the VH sequence:

(SEQ ID NO: 5)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSS.

(4) A humanized antibody comprising a full-length light (L) chain having the sequence of SEQ ID NO:6, and a full-length heavy (H) chain having the sequence of one of SEQ ID NO:7, SEQ ID NO:8, or SEQ ID NO:15, wherein the sequences are indicated below.

(5) A humanized antibody comprising a full-length light (L) chain having the sequence of SEQ ID NO:9, and a full-length heavy (H) chain having the sequence of one of SEQ ID NO:10, SEQ ID NO:11, SEQ ID NO:12, SEQ ID NO:13, or SEQ ID NO:14, wherein the sequences are indicated below.

SEQ ID NO: 6:
DIGMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 7:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 8:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 9:
DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 10:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 11:
EVQLVESGGGLVQPGGSLRLSCAASGYTFTSYNMHWVRQAPGKGLEWVGA
IYPGNGATSYNQKEKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCAVSNKALPAPIEATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 12:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYEPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 13:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHWHYTQKSLSLSP
GK
SEQ ID NO: 14:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 15:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

The murine anti-human CD20 antibody, m2H7, has the sequences:

VL sequence:

(SEQ ID NO: 24)

QIVLSQSPAI LSASPGEKVT MTCRASSSVS YMHWYQQKPG
SSPKPWIYAP SNLASGVPARFSGSGSGTSY SLTISRVEAE
DAATYYCQQW SFNPPTFGAG TKLELK

VH sequence:

(SEQ ID NO: 25)

QAYLQQSGAE LVRPGASVKM SCKASGYTFT SYNMHWVKQT
PRQGLEWIGA IYPGNGDTSY NQKFKGKATL TVDKSSSTAY
MQLSSLTSED SAVYFCARVV YYSNSYWYFD VWGTGTTVTV S

In the B-cell-surface marker-binding antibodies that comprise an Fc region, the C-terminal lysine (residue 447 according to the EU numbering system) of the Fc region may be removed, for example, during purification of the antibody or by recombinantly engineering the nucleic acid encoding the antibody polypeptide. For example, 2H7 or another humanized antibody herein can comprise an Fc region including the K447 residue, or with all the K447 residues removed, or a mixture of antibodies having Fc regions with and without the K447 residue.

In certain embodiments, the humanized antibody useful herein further comprises amino acid alterations in the IgG Fc and exhibits increased binding affinity for human FcRn over an antibody having wild-type IgG Fc, by at least about 60 fold, at least about 70 fold, at least about 80 fold, and more preferably at least about 100 fold, still more preferably at least about 125 fold, and even more preferably at least about 150 fold to about 170 fold.

The N-glycosylation site in IgG is at Asn297 in the CH2 domain. Included for use in therapy herein are compositions of any humanized antibodies having an Fc region, wherein about 80-100% (and preferably about 90-99%) of the antibody in the composition comprises a mature core carbohydrate structure that lacks fucose, attached to the Fc region of the glycoprotein, or has reduced fucose content.

A “bispecific humanized antibody” encompasses an antibody wherein one arm of the antibody has at least the antigen binding region of the H and/or L chain of a humanized antibody of the invention, and the other arm has V-region binding specificity for a second antigen. In specific exemplary embodiments, the second antigen is selected from the group consisting of CD3, CD64, CD32A, CD16, NKG2D, or other NK-activating ligands.

The terms “BAFF,” “BAFF polypeptide,” “TALL-1” or “TALL-1 polypeptide,” “BLyS,” and “THANK” when used herein encompass “native-sequence BAFF polypeptides” and “BAFF variants.” “BAFF” is a designation given to those polypeptides that have the human BAFF sequence as set forth in, for example, US 2006/0110387, and homologs and fragments and variants thereof, which have the biological activity of the native-sequence BAFF. A biological activity of BAFF can be selected from the group consisting of promoting B-cell survival, promoting B-cell maturation, and binding to BR3. The term “BAFF” includes those polypeptides described in Shu et al., J. Leukocyte Biol., 65:680 (1999); GenBank Accession No. AF136293; WO 1998/18921; EP 869,180; WO 1998/27114; WO 1999/12964; WO 1999/33980; Moore et al., Science, 285:260-263 (1999); Schneider et al., J. Exp. Med., 189:1747-1756 (1999); and Mukhopadhyay et al., J. Biol. Chem., 274:15978-15981 (1999).

The term “BAFF antagonist” as used herein is used in the broadest sense, and includes any molecule that (1) binds a native-sequence BAFF polypeptide or binds a native-sequence BR3 polypeptide to block, partially or fully, BR3 interaction with a BAFF polypeptide, and (2) partially or fully blocks, inhibits, or neutralizes native-sequence BAFF signaling. Native-sequence BAFF polypeptide signaling promotes, among other things, B-cell survival and B-cell maturation. The inhibition, blockage, or neutralization of BAFF signaling results in, inter alia, a reduction in the number of B cells. A BAFF antagonist as defined herein will partially or fully block, inhibit, or neutralize one or more biological activities of a BAFF polypeptide, in vitro or in vivo. In one embodiment, a biologically active BAFF potentiates any one or a combination of the following events in vitro or in vivo: an increased survival of B cells, an increased level of IgG and/or IgM, an increased number of plasma cells, and processing of NF-κb2/100 to p52 NF-κb in splenic B cells (see, e.g., Batten et al., J. Exp. Med., 192:1453-1465 (2000); Moore et al., Science, 285:260-263 (1999); and Kayagaki et al., Immunity, 10:515-524 (2002)).

In some embodiments, a BAFF antagonist as defined herein includes anti-BAFF antibodies, BAFF-binding polypeptides (including immunoadhesins and peptides), and BAFF-binding small molecules. BAFF antagonists include, for example, the BAFF-binding antibodies described in WO 2002/02641 (e.g., antibodies comprising the amino acid sequence of any of SEQ ID NOS:1-46, 321-329, 834-872, 1563-1595, 1881-1905 of Table 1 thereof). In a further embodiment, the immunoadhesin comprises a BAFF-binding region of a BAFF receptor (e.g., an extracellular domain of BR3, BCMA, or TACI). In a still further embodiment, the immunoadhesin is BR3-Fc. Other examples of BAFF-binding Fc proteins can be found in WO 2002/66516, WO 2000/40716, WO 2001/87979, WO 2003/024991, WO 2002/16412, WO 2002/38766, WO 2002/092620, and WO 2001/12812. Methods of making BAFF antagonists are described, for example, in US 2005/0095243 and US 2005/0163775.

The terms “BR3” and “BR3 polypeptide” when used herein encompass native-sequence BR3 polypeptides and BR3 variants, as defined hereinbelow. “BR3” is a designation given to those polypeptides comprising, for example, the human BR3 sequence set forth in WO 2003/14294 and US 2005/0070689.

The BR3 polypeptides of the invention can be isolated from a variety of sources, such as from human tissue types or from another source, or prepared by recombinant and/or synthetic methods. The term BR3 includes the BR3 polypeptides described in WO 2002/24909, WO 2003/14294, and US 2005/0070689. Anti-BR3 antibodies can be prepared in accordance with methods set forth in, for example, WO 2003/14294 and US 2005/0070689.

A “native-sequence” BR3 polypeptide or “native BR3” comprises a polypeptide having the same amino acid sequence as the corresponding BR3 polypeptide derived from nature. Such native-sequence BR3 polypeptides can be isolated from nature or can be produced by recombinant and/or synthetic means. The term “native-sequence BR3 polypeptide” specifically encompasses naturally occurring truncated, soluble, or secreted forms (e.g., an extracellular domain sequence), naturally occurring variant forms (e.g., alternatively spliced forms), and naturally occurring allelic variants of the polypeptide. The BR3 polypeptides of the invention include the BR3 polypeptide comprising or consisting of the contiguous sequence of amino acid residues 1 to 184 of a human BR3 (see WO 2003/14294 and US 2005/0070689).

A BR3 “extracellular domain” or “ECD” refers to a form of the BR3 polypeptide that is essentially free of the transmembrane and cytoplasmic domains. ECD forms of BR3 include a polypeptide comprising any one of the amino acid sequences selected from the group consisting of amino acids 1-77, 2-62, 2-71, 1-61, 7-71, 23-38, and 2-63 of human BR3. The invention contemplates BAFF antagonists that are polypeptides comprising any one of the above-mentioned ECD forms of human BR3 and variants and fragments thereof that bind a native BAFF.

“BR3 variant” means a BR3 polypeptide having at least about 80% amino acid sequence identity with the amino acid sequence of a native-sequence, full-length BR3 or BR3 ECD and binds a native-sequence BAFF polypeptide. Optionally, the BR3 variant includes a single cysteine-rich domain. Such BR3 variant polypeptides include, for instance, BR3 polypeptides wherein one or more amino acid residues are added, or deleted, at the N- and/or C-terminus, as well as within one or more internal domains, of the full-length amino acid sequence. Fragments of the BR3 ECD that bind a native-sequence BAFF polypeptide are also contemplated. According to one embodiment, a BR3 variant polypeptide will have at least about 80% amino acid sequence identity, at least about 81% amino acid sequence identity, at least about 82% amino acid sequence identity, at least about 83% amino acid sequence identity, at least about 84% amino acid sequence identity, at least about 85% amino acid sequence identity, at least about 86% amino acid sequence identity, at least about 87% amino acid sequence identity, at least about 88% amino acid sequence identity, at least about 89% amino acid sequence identity, at least about 90% amino acid sequence identity, at least about 91% amino acid sequence identity, at least about 92% amino acid sequence identity, at least about 93% amino acid sequence identity, at least about 94% amino acid sequence identity, at least about 95% amino acid sequence identity, at least about 96% amino acid sequence identity, at least about 97% amino acid sequence identity, at least about 98% amino acid sequence identity, or at least about 99% amino acid sequence identity with a human BR3 polypeptide or a specified fragment thereof (e.g., ECD). BR3 variant polypeptides do not encompass the native BR3 polypeptide sequence. According to another embodiment, BR3 variant polypeptides are at least about 10 amino acids in length, at least about 20 amino acids in length, at least about 30 amino acids in length, at least about 40 amino acids in length, at least about 50 amino acids in length, at least about 60 amino acids in length, or at least about 70 amino acids in length.

The term “APRIL antagonist” as used herein is used in the broadest sense, and includes any molecule that (1) binds a native-sequence APRIL polypeptide or binds a native-sequence ligand to APRIL to block, partially or fully, the ligand's interaction with APRIL polypeptide, and (2) partially or fully blocks, inhibits, or neutralizes native-sequence APRIL signaling. Native-sequence APRIL polypeptide signaling promotes, among other things, B-cell survival and B-cell maturation. APRIL (a proliferation-inducing ligand) is a TNF family member with a shared receptor to BAFF. Examples of preferred APRIL antagonists include atacicept (same as TACI-Ig immunoadhesin) and a BAFF/APRIL antagonist (soluble BCMA-Fc).

As used herein, “rheumatoid arthritis” or “RA” refers to a recognized disease state that may be diagnosed according to the 2000 revised American Rheumatoid Association criteria for the classification of RA, or any similar criteria. The term includes not only active and early RA, but also incipient RA, as defined below. Physiological indicators of RA include symmetric joint swelling, which is characteristic though not invariable in RA. Fusiform swelling of the proximal interphalangeal (PIP) joints of the hands as well as metacarpophalangeal (MCP), wrists, elbows, knees, ankles, and metatarsophalangeal (MTP) joints are commonly affected and swelling is easily detected. Pain on passive motion is the most sensitive test for joint inflammation, and inflammation and structural deformity often limits the range of motion for the affected joint. Typical visible changes include ulnar deviation of the fingers at the MCP joints, hyperextension, or hyperflexion of the MCP and PIP joints, flexion contractures of the elbows, and subluxation of the carpal bones and toes. The subject with RA may be resistant to DMARDs, in that the DMARDs are not effective or fully effective in treating symptoms. Further candidates for therapy according to this invention include those who have experienced an inadequate response to previous or current treatment with TNF inhibitors such as etanercept, infliximab, and/or adalimumab because of toxicity or inadequate efficacy (for example, etanercept for 3 months at 25 mg twice a week or at least 4 infusions of infliximab at 3 mg/kg). RA includes, for example, juvenile-onset RA, juvenile idiopathic arthritis (JIA), or juvenile RA (JRA).

A patient with “active rheumatoid arthritis” means a patient with active and not latent symptoms of RA. Subjects with “early active rheumatoid arthritis” are those with active RA diagnosed for at least eight weeks but no longer than four years, according to the revised 1987 ACR criteria for the classification of RA. Subjects with “early rheumatoid arthritis” are those subjects with RA diagnosed for at least eight weeks but no longer than four years, according to the revised 1987 ACR criteria for classification of RA.

Patients with “incipient RA” have early polyarthritis that does not fully meet ACR criteria for a diagnosis of RA, in association with the presence of RA-specific prognostic biomarkers such as anti-CCP and SE. They include patients with positive anti-CCP who present with polyarthritis, but do not yet have a diagnosis of RA, and are at high risk for going on to develop bona fide ACR criteria RA (95% probability).

“Joint damage” is used in the broadest sense and refers to damage or partial or complete destruction to any part of one or more joints, including the connective tissue and cartilage, where damage includes structural and/or functional damage of any cause, and may or may not cause joint pain/arthalgia. It includes, without limitation, joint damage associated with or resulting from inflammatory joint disease as well as non-inflammatory joint disease. This damage may be caused by any condition, such as an autoimmune disease, especially arthritis, and most especially RA. Exemplary such conditions include acute and chronic arthritis, RA including juvenile-onset RA, juvenile idiopathic arthritis (JIA), or juvenile RA (JRA), and stages such as rheumatoid synovitis, gout or gouty arthritis, acute immunological arthritis, chronic inflammatory arthritis, degenerative arthritis, type II collagen-induced arthritis, infectious arthritis, septic arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), rheumatic autoimmune disease other than RA, and significant systemic involvement secondary to RA (including but not limited to vasculitis, pulmonary fibrosis, or Felty's syndrome). For purposes herein, joints are points of contact between elements of a skeleton (of a vertebrate such as an animal) with the parts that surround and support it and include, but are not limited to, for example, hips, joints between the vertebrae of the spine, joints between the spine and pelvis (sacroiliac joints), joints where the tendons and ligaments attach to bones, joints between the ribs and spine, shoulders, knees, feet, elbows, hands, fingers, ankles and toes, but especially joints in the hands and feet.

“Treatment” of a subject herein refers to both therapeutic treatment and prophylactic or preventative measures. Those in need of treatment include those already with RA or joint damage as well as those in which the RA or joint damage or the progress of RA or joint damage is to be prevented. Hence, the subject may have been diagnosed as having the RA or joint damage or may be predisposed or susceptible to the RA or joint damage, or may have RA or joint damage that is likely to progress in the absence of treatment. Treatment is successful herein if the RA or joint damage is alleviated or healed, or progression of RA or joint damage, including its signs and symptoms and structural damage, is halted or slowed down as compared to the condition of the subject prior to administration. Successful treatment further includes complete or partial prevention of RA or of the development of joint or structural damage. For purposes herein, slowing down or reducing RA or joint damage or the progression of joint damage is the same as arrest, decrease, or reversal of the RA or joint damage.

As used herein, the term “patient” refers to any single animal, more preferably a mammal (including humans and such non-human animals as, e.g., dogs, cats, horses, rabbits, zoo animals, cows, pigs, sheep, and non-human primates), for which treatment is desired. Most preferably, the patient herein is a human.

A “subject” herein is any single human subject, including a patient, eligible for treatment who is experiencing or has experienced one or more signs, symptoms, or other indicators of RA or joint damage, whether, for example, newly diagnosed or previously diagnosed and now experiencing a recurrence or relapse, or is at risk for RA or joint damage, no matter the cause. Intended to be included as a subject are any subjects involved in clinical research trials not showing any clinical sign of disease, or subjects involved in epidemiological studies, or subjects once used as controls. The subject may have been previously treated with a medicament for RA or joint damage, including a B-cell antagonist, or not so treated. The subject may be naïve to a second medicament being used when the treatment herein is started, i.e., the subject may not have been previously treated with, for example, an immunosuppressive agent such as MTX at “baseline” (i.e., at a set point in time before the administration of a first dose of antagonist in the treatment method herein, such as the day of screening the subject before treatment is commenced). Such “naïve” subjects are generally considered to be candidates for treatment with such second medicament.

“Clinical improvement” refers to prevention of further progress of RA or joint damage or any improvement in RA or joint damage as a result of treatment, as determined by various testing, including radiographic testing. Thus, clinical improvement may, for example, be determined by assessing the number of tender or swollen joints, performing the Psoriasis Assessment Severity Index, performing a global clinical assessment of the subject, assessing erythrocyte sedimentation rate, or assessing the amount of C-reactive protein level.

For purposes herein, a subject is in “remission” if he/she has no symptoms of RA or active joint damage, such as those detectable by the methods disclosed herein, and has had no progression of RA or joint damage as assessed at baseline or at a certain point of time during treatment. Those who are not in remission include, for example, those experiencing a worsening or progression of RA or joint damage. Such subjects experiencing a return of symptoms, including active RA or joint damage, are those who have “relapsed” or had a “recurrence.”

A “symptom” of RA or joint damage is any morbid phenomenon or departure from the normal in structure, function, or sensation, experienced by the subject and indicative of RA or joint damage, such as those noted above, including tender or swollen joints.

The expression “effective amount” refers to an amount of a medicament that is effective for treating RA or joint damage. This would include an amount that is effective in achieving a reduction in RA or joint damage as compared to baseline prior to administration of such amount as determined, e.g., by radiographic or other testing. An effective amount of a second medicament may serve not only to treat the RA or joint damage in conjunction with the antagonist herein, but also serve to treat undesirable effects, including side-effects or symptoms or other conditions accompanying RA or joint damage, including a concomitant or underlying disease or disorder.

“Total modified Sharp score” means a score obtained for assessment of radiographs using the method according to Sharp, as modified by Genant, Am. J. Med., 30:35-47 (1983). The primary assessment will be the change in the total Sharp-Genant score from screening. The Sharp-Genant score combines an erosion score and a joint space narrowing score of both hands and feet. Joint damage is measured in this test scoring by a mean change of less than the score at baseline (when patient is screened or tested before first administration of the antagonist herein).

A “chemotherapeutic agent” is a chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide, and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as MTX and 5-fluorouracil (5-FU); folic acid analogues such as denopterin, MTX, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; Xeloda; ibandronate; CPT-11; topoisomerase inhibitor RFS 2000; difluoromethylornithine (DFMO); retinoic acid; esperamicins; capecitabine; and pharmaceutically acceptable salts, acids or derivatives of any of the above.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as MTX (oral or subcutaneous); anti-malarial agents such as chloroquine and hydroxychloroquine; sulfasalazine; leflunomide; cytokine antagonists such as cytokine antibodies or cytokine receptor antibodies including anti-interferon-α, -β, or -γ antibodies, anti-TNF-α antibodies (infliximab (REMICADE®) or adalimumab), anti-TNF-α immunoadhesin (etanercept), anti-TNF-β antibodies, anti-interleukin-2 (IL-2) antibodies and anti-IL-2 receptor antibodies, and anti-IL-6 receptor antibodies and antagonists (such as ACTEMRA™ (tocilizumab); see also WO 2004/096273); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187); streptokinase; transforming growth factor-β (TGF-β); streptodornase; RNA or DNA from the host; FK506; RS-61443; chlorambucil; deoxyspergualin; rapamycin; T-cell receptor (U.S. Pat. No. 5,114,721); T-cell receptor fragments (Offner et al., Science, 251:430-432 (1991); WO 90/11294; Janeway, Nature, 341:482-483 (1989); and WO 91/01133); BAFF antagonists such as anti-BAFF antibodies and anti-BR3 antibodies and zTNF4 antagonists (for review, see Mackay and Mackay, Trends Immunol., 23:113-115 (2002)); biologic agents that interfere with T cell helper signals, such as anti-CD40 receptor or anti-CD40 ligand (CD154), including blocking antibodies to CD40-CD40 ligand (e.g., Durie et al., Science, 261:1328-1330 (1993); Mohan et al., J. Immunol., 154:1470-1480 (1995)) and CTLA4-Ig (Finck et al., Science, 265:1225-1227 (1994)); and T-cell receptor antibodies (EP 340,109) such as T10B9. Some immunosuppressive agents herein are also DMARDs, such as MTX. Examples of preferred immunosuppressive agents herein include cyclophosphamide, chlorambucil, azathioprine, leflunomide, MMF, or MTX.

The term “cytokine” is a generic term for proteins released by one cell population that act on another cell as intercellular mediators. Examples of such cytokines are lymphokines, monokines; interleukins such as IL-1, IL-1α, IL-2, IL-3, IL-4, IL-5, IL-6, IL-7, IL-8, IL-9, IL-11, IL-12, IL-15, including PROLEUKIN® rIL-2; a TNF such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof. A “cytokine antagonist” is a molecule that inhibits or antagonizes such cytokines by any mechanism, including, e.g., antibodies to the cytokine, antibodies to the cytokine receptor, and immunoadhesins.

The term “integrin” refers to a receptor protein that allows cells both to bind to and to respond to the extracellular matrix and is involved in a variety of cellular functions such as wound healing, cell differentiation, homing of tumor cells, and apoptosis. They are part of a large family of cell adhesion receptors that are involved in cell-extracellular matrix and cell-cell interactions. Functional integrins consist of two transmembrane glycoprotein subunits, called alpha and beta, which are non-covalently bound. The α subunits all share some homology to each other, as do the β subunits. The receptors always contain one a chain and one β chain. Examples include α6μ1, α3μ1, α7β1, the α4 chain such as α4μ1, the β7 chain such as the β7 integrin subunit of α4β7 and/or αEP7, LFA-1 etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

An “integrin antagonist” is a molecule that inhibits or antagonizes such integrins by any mechanism, including, for example, antibodies to the integrin. Examples of “integrin antagonists or antibodies” herein include an LFA-1 antibody, such as efalizumab (RAPTIVA®) commercially available from Genentech, or other CD11/11a and CD18 antibodies, or an α4 integrin antibody such as natalizumab (ANTEGREN®) available from Biogen-IDEC, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. No. 6,677,339 and U.S. Pat. No. 6,348,463), aromatic amine derivatives (U.S. Pat. No. 6,369,229), ADAM disintegrin domain polypeptides (US 2002/0042368), antibodies to alphavbeta3 integrin (EP 633945), anti-beta7 antibodies such as rhuMAb Beta7 (US 2006/0093601) and MLN-02 (Millennium Pharmaceuticals), anti-alpha4 antibodies such as TYSABRI® (Biogen-IDEC-Elan), T0047 (GSK/Tanabe), CDP-323 (oral) (UCB), aza-bridged bicyclic amino acid derivatives (WO 2002/02556), etc.

For purposes herein, “tumor necrosis factor-alpha” or “TNF-α” refers to a human TNF-α molecule comprising the amino acid sequence as described in Pennica et al., Nature, 312:721 (1984) or Aggarwal et al., JBC, 260:2345 (1985). A “TNF-α inhibitor” herein is an agent that inhibits, to some extent, a biological function of TNF-α, generally through binding to TNF-α and neutralizing its activity. Examples of TNF-α inhibitors herein include antibodies and immunoadhesins such as etanercept (ENBREL®), infliximab (REMICADE®), and adalimumab (HUMIRA™).

Examples of “disease-modifying anti-rheumatic drugs” or “DMARDs” include hydroxycloroquine, sulfasalazine, MTX, leflunomide, etanercept, infliximab (optionally together with oral or subcutaneous MTX), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197(9):1125-1139 (2003)), including salts and derivatives thereof, etc. A preferred DMARD herein is MTX.

Examples of “non-steroidal anti-inflammatory drugs” or “NSAIDs” include aspirin, acetylsalicylic acid, ibuprofen, naproxen, indomethacin, sulindac, tolmetin, COX-2 inhibitors such as celecoxib (CELEBREX®; 4-(5-(4-methylphenyl)-3-(trifluoromethyl)-1H-pyrazol-1-yl) benzenesulfonamide and valdecoxib (BEXTRA®), and meloxicam (MOBIC®), including salts and derivatives thereof, etc. Preferably, they are aspirin, naproxen, ibuprofen, indomethacin, or tolmetin.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

A “medicament” is an active drug to treat RA or joint damage or the signs or symptoms or side effects of RA or joint damage.

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

A “kit” is any article of manufacture (e.g., a package or container) comprising at least one reagent, e.g., a medicament for treatment of RA or joint damage, or a probe for specifically detecting a biomarker gene or protein of the invention. The article of manufacture is preferably promoted, distributed, or sold as a unit for performing the methods of the present invention.

A “target audience” is a group of people or an institution to whom or to which a particular medicament is being promoted or intended to be promoted, as by marketing or advertising, especially for particular uses, treatments, or indications, such as individual patients; patient populations; readers of newspapers, medical literature, and magazines; television or internet viewers; radio or internet listeners; physicians; drug companies; etc.

The term “sample” shall generally mean any biological sample obtained from an individual, body fluid, body tissue, cell line, tissue culture, or other source. Body fluids are, e.g., lymph, sera, whole fresh blood, peripheral blood mononuclear cells, frozen whole blood, plasma (including fresh or frozen), urine, saliva, semen, synovial fluid, and spinal fluid. Samples also include synovial tissue, skin, hair follicle, and bone marrow. Methods for obtaining tissue biopsies and body fluids from mammals are well known in the art. If the term “sample” is used alone, it shall still mean that the “sample” is a “biological sample”, i.e., the terms are used interchangeably.

A “genetic sample” is a sample containing genetic material such as nucleic acids, especially DNA. Typically, the genetic material can be extracted from the sample by conventional means and analyzed for polymorphisms and alleles to determine the presence or expression of biomarkers. Genetic samples include blood and other body fluids as well as tissues and cells.

The term “biomarker” as used in the present application refers generally to a DNA, RNA, protein, carbohydrate, or glycolipid-based molecular marker, the expression or presence of which in a subject's sample can be detected by standard methods (or methods disclosed herein) and is predictive of the effective responsiveness or sensitivity of a mammalian subject with RA to a B-cell antagonist. Such biomarkers contemplated by the present invention include, but are not limited to, PTPN22 R620W SNP or SE, or both. They may also include anti-CCP and RF and other biomarkers. The terms “marker” and “biomarker” are used herein interchangeably.

“Shared epitope” or “SE” or “rheumatoid epitope” as used herein means the sequence motifs in residues 70 to 74 of the third hypervariable region of the HLA-DRB1 chain encoded by the HLA-DRB1*0401, *0404/0408, *0405, *0409, *0410, *0413, *0416, *0101, *0102, *0104, *1001, *1402, and *1406 alleles in the predisposition to RA. Specifically, the sequence motifs are characterized by the amino acid coding sequence QKRAA (SEQ ID NO:26) or QRRAA (SEQ ID NO:27) or RRRAA (SEQ ID NO:28) in the third hypervariable region, encompassing amino acid residues 70 to 74 of the HLA-DRB1 chain of the major histocompatibility complex class II molecule. Because DNA typing examines the alleles at a given locus, the name of the locus precedes the designation of the specific allele (with the two terms separated by an asterisk); for example, HLA-DRB1*0401 refers to the 0401 allele of the HLA-DRB1 locus. One particular HLA-DR specificity is encoded by several HLA-DRB1 alleles in conjunction with the product of the HLA-DRA1 locus; for example, more than 11 HLA-DRB1 alleles (HLA-DRB1*0401 to *0411) can encode the B chain of the HLA-DR4 specificity. For purposes herein, responsiveness to treatment of RA with a B-cell antagonist is positively correlated with the incidence or presence of this genetic biomarker in patients with alleles for SE that are homozygous or heterozygous.

“PTPN22 R620W single-nucleotide polymorphism” or “PTPN22 R620W SNP” as used herein refers to a variation at position 620 of the amino acid sequence of PTPN22, which is an intracellular protein of about 105-kD with a single tyrosine phosphatase catalytic domain. This allelic variation is changing an arginine to a tryptophan, which causes a variation in the corresponding encoded gene from CT to TT at position 1858 of the corresponding polynucleotide. The “PTPN22 CT/TT genotype” as used herein refers to that genetic variation. For purposes herein, responsiveness to treatment of RA with a B-cell antagonist is positively linked to the incidence or presence of this genetic biomarker in patients with alleles for the PTPN22 CT/TT genotype that are homozygous or heterozygous.

“Rheumatoid factor” or “RF” is an immunoglobulin directed against the Fc portion of another immunoglobulin commonly used as a blood test for the diagnosis of RA. It can self-aggregate into a lattice-like form within joint cavities to provide a surface onto which inflammatory cells can adhere and act. RA patients with a high titer of RF (approximately 80% of patients) have more aggressive disease, with a worse long-term outcome and increased mortality over those who are RF negative.

“Anti-cyclic citrullinated peptide” or “CCP” antibodies are antibodies to peptides in which arginine has been post-translationally modified to become citrulline. These autoantibodies are strongly correlated with, but may represent distinct clinical subsets of, RA.

The verbs “determine” and “assess” shall have the same meaning and are used interchangeably throughout the application.

The “expression level” associated with an increased clinical benefit to a RA patient or patient with joint damage is a detectable level in a biological sample. These can be measured by methods known to the expert skilled in the art and also disclosed by this invention. The expression level or amount of biomarker assessed can be used to determine the response to the treatment.

“Seropositivity” as used herein means showing a positive reaction to a test on blood serum indicated by the presence of a certain autoantibody or biomarker in the blood sample.

An “effective response” of a patient or a patient's “responsiveness” to treatment with a B-cell antagonist and similar wording refers to the clinical or therapeutic benefit imparted to a patient (that patient being at risk for or suffering from RA) from or as a result of the treatment with the antagonist, such as an anti-CD20, anti-CD22, or anti-BR3 antibody or BR3-Fc immunoadhesin. Such benefit includes cellular or biological responses, a complete response, a partial response, a stable disease (without progression or relapse), or a response of the patient from or as a result of the treatment with the antagonist with a later relapse. For example, an effective response can be a higher ACR50 in an anti-CD20 antibody-treated patient diagnosed with one or both of the genetic biomarkers herein versus a similarly treated patient not diagnosed with one or both of the biomarkers. The incidence of genetic biomarker(s) herein effectively predicts, or predicts with high sensitivity, such effective response.

The expression “not responsive to,” as it relates to the reaction of subjects or patients to one or more of the medicaments that were previously administered to them, describes those subjects or patients who, upon administration of such medicament(s), did not exhibit any or adequate signs of treatment of the disorder for which they were being treated, or they exhibited a clinically unacceptably high degree of toxicity to the medicament(s), or they did not maintain the signs of treatment after first being administered such medicament(s), with the word “treatment” being used in this context as defined herein. The phrase “not responsive” includes a description of those subjects who are resistant and/or refractory to the previously administered medication(s), and includes the situations in which a subject or patient has progressed while receiving the medicament(s) that he or she is being given, and in which a subject or patient has progressed within 12 months (for example, within six months) after completing a regimen involving the medicament(s) to which he or she is no longer responsive. The non-responsiveness to one or more medicaments thus includes subjects who continue to have active disease following previous or current treatment therewith. For instance, a patient may have active disease activity after about one to three months of therapy with the medicament(s) to which he/she is non-responsive. Such responsiveness may be assessed by a clinician skilled in treating the disorder in question.

For purposes of non-response to medicament(s), a subject who experiences “a clinically unacceptably high level of toxicity” from previous or current treatment with one or more medicaments experiences one or more negative side-effects or adverse events associated therewith that are considered by an experienced clinician to be significant, such as, for example, serious infections, congestive heart failure, demyelination (leading to MS), significant hypersensitivity, neuropathological events, high degrees of autoimmunity, a cancer such as endometrial cancer, non-Hodgkin's lymphoma, breast cancer, prostate cancer, lung cancer, ovarian cancer, or melanoma, tuberculosis (TB), etc.

By “reducing the risk of a negative side effect” is meant reducing the risk of a side effect resulting from treatment with the antagonist herein to a lower extent than the risk observed resulting from treatment of the same patient or another patient with a previously administered medicament. Such side effects include those set forth above regarding toxicity, and are preferably infection, cancer, heart failure, or demyelination.

The word “detectable label” when used herein refers to a compound or composition that is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition that is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The terms “level of expression” or “expression level” are used interchangeably and generally refer to the amount of a polynucleotide or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even post-translational modification of the protein. Fragments of the transcribed polynucleotide, the translated protein, or the post-translationally modified protein shall also be regarded as expressed, whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “covariate” refers to certain variables or information relating to a patient. The clinical endpoints are frequently considered in regression models, where the endpoints represent the dependent variable and the biomarkers represent the main or target independent variables (regressors). If additional variables from the clinical data pool are considered, they are denoted as (clinical) covariates.

The term “clinical covariate” is used herein to describe all clinical information about the patient, which is in general available at baseline. These clinical covariates comprise demographic information like sex, age, etc., other anamnestic information, concomitant diseases, concomitant therapies, results of physical examinations, common laboratory parameters obtained, known properties of the RA or joint damage, information quantifying the extent of RA disease, clinical performance scores like ECOG or Karnofsky index, clinical disease staging, timing and result of pretreatments, disease history, as well as all similar information that may be associated with the clinical response to treatment.

As used herein, the term “raw analysis” or “unadjusted analysis” refers to regression analyses, wherein besides the considered biomarkers, no additional clinical covariates are used in the regression model, neither as independent factors nor as stratifying covariate.

As used herein, the term “adjusted by covariates” refers to regression analyses, wherein besides the considered biomarkers, additional clinical covariates are used in the regression model, either as independent factors or as stratifying covariate.

As used herein, the term “univariate” refers to regression models or graphical approaches wherein, as an independent variable, only one of the target biomarkers is part of the model. These univariate models can be considered with and without additional clinical covariates.

As used herein, the term “multivariate” refers to regression models or graphical approaches wherein, as independent variables, more than one of the target biomarkers is part of the model. These multivariate models can be considered with and without additional clinical covariates.

### B. Modes for Carrying Out the Invention

The present invention provides a method for identifying patients whose RA or joint damage is likely to be responsive to B-cell antagonist therapy. The method is useful, inter alia, for increasing the likelihood that administration of a B-cell antagonist to a patient with RA or joint damage will be efficacious.

The methods and assays disclosed herein are directed to the examination of expression of one or two genetic biomarkers in a biological sample, wherein the determination of that expression is predictive or indicative of whether the sample will be sensitive to B-cell antagonists such as antibodies or immunoadhesins.

The disclosed methods and assays provide for convenient, efficient, and potentially cost-effective means to obtain data and information useful in assessing appropriate or effective therapies for treating patients. For example, a patient having been diagnosed with RA could provide a blood sample or synovial fluid and the sample could be examined by way of various in vitro assays to determine whether the patient's cells would be sensitive to a therapeutic agent that is a B-cell antagonist, such as an anti-CD20, anti-CD22, or anti-BR3 antibody.

I. Diagnostics

The invention provides methods for predicting the sensitivity of a sample to a B-cell antagonist. The methods may be conducted in a variety of assay formats, including assays detecting genetic or protein expression (such as PCR and enzyme immunoassays) and biochemical assays detecting appropriate activity. Determination of expression or the presence of such biomarkers in the samples is predictive that the patient providing the sample will be sensitive to the biological effects of a B-cell antagonist. The invention herein is that the expression of the SNP herein or SE or both in a sample from a RA patient would indicate that such patient would exhibit better efficacy upon treatment with a B-cell antagonist than a similarly situated patient without such genetic expression.

In one aspect, this invention provides a method of determining whether a patient with RA will respond effectively to treatment with a B-cell antagonist, comprising assessing, as a biomarker, genetic expression of a PTPN22 R620W SNP and/or SE in a sample from the patient. In addition, the method optionally also comprises assessing other biomarkers, including seropositivity for one or both the biomarkers anti-CCP and RF, in a sample from the patient. The presence of PTPN22 CT/TT genotype and/or SE alone or in combination with other biomarkers such as seropositivity for one or both of the biomarkers anti-CCP and RF shows that a patient will respond effectively to treatment with the antagonist.

According to this method, a biological sample is obtained from the patient and subjected to an assay to evaluate whether PTPN22 CT/TT genotype and/or SE are present in the sample. In one preferred alternative, the presence of the genotype and/or SE is evaluated without any other biomarkers. In another preferred alternative, other biomarkers are assessed. For example, seropositivity for one or both of anti-CCP antibodies and RF also may be detected and used in combination with the genetic markers to predict effective response to the B-cell antagonist. Where the genotype and/or SE are detected, with or without the other biomarkers, the patient is determined to be eligible for treatment with a B-cell antagonist.

Other biomarkers besides the four mentioned above that can be used for monitoring effective response of a patient to a B-cell antagonist treatment include C-reactive protein (CRP), serum amyloid A (SAA), S100 (e.g. S100A12), osteopontin, matrix metalloprotease 1 (MMP-1), anti-agalactosyl IgG antibodies (CARF), a pro-form of MMP-1 such as pro-MMP, matrix metalloprotease 3 (MMP-3), HA, sCD14, anti-nuclear autoantibodies (ANA), anti-double-stranded DNA antibodies, antibodies to extractable nuclear antigens (ENA), anti-neutrophil cytoplasmic autoantibodies (ANCA), anti-keratin antibodies (AKA), anti-filaggrin antibodies (AFA), angiogenesis markers, and products of bone, cartilage or synovium metabolism. In addition, cytokines can be biomarkers, such as, for example, IFN-γ, IL-1β, TNF-α, G-CSF, GM-CSF, IL-6, IL-4, IL-10, IL-13, IL-5, CCL4/MIP-1β, IL-7, IL-2, GM-CSF, G-CSF, CCL2/MCP-1, EGF, VEGF, CXCL8/IL-8, IL-12, IL-17, as well as erythrocyte sedimentation rate and joint counts compared to the severe RA groups.

Assessment of the single- or dual-marker expression level of SE and/or genotype, without more, would be expected to provide an accurate prediction of level of sensitivity of the patient to a B-cell antagonist.

One of skill in the medical arts, particularly pertaining to the application of diagnostic tests and treatment with therapeutics, will recognize that biological systems are somewhat variable and not always entirely predictable, and thus many good diagnostic tests or therapeutics are occasionally ineffective. Thus, it is ultimately up to the judgment of the attending physician to determine the most appropriate course of treatment for an individual patient, based upon test results, patient condition and history, and his or her own experience. There may even be occasions, for example, when a physician will choose to treat a patient with a B-cell antagonist even when a patient is not predicted to be particularly sensitive to B-cell antagonists, based on data from diagnostic tests or from other criteria, particularly if all or most of the other obvious treatment options have failed, or if some synergy is anticipated when given with another treatment. The fact that the anti-CD20 antibodies, for example, as a class of drugs are relatively well tolerated compared to more traditional immunosuppressive agents used in the treatment of RA makes this a more viable option.

Furthermore, this invention also provides additional methods wherein simultaneous assessment of the expression levels in patient samples of biomarkers in addition to SE and/or SNP genotype is carried out. In preferred embodiments of these methods there is a reduced possibility of false prediction, due to the number of markers of which expression levels are assessed.

The presence of one of these two biomarkers (PTPN22 R620W SNP genotype and SE), or the simultaneous presence of both of these biomarkers, equates with high sensitivity to treatment with a B-cell antagonist. In a preferred embodiment of this method, the biomarkers comprise SE and/or the genotype, as well as anti-CCP and/or RF, wherein the presence of the SE and/or genotype along with seropositivity for one or more of anti-CCP and RF indicate high sensitivity of the patient to treatment with a B-cell antagonist. This is a matrix that could involve genotype, genotype plus RF, genotype plus anti-CCP, genotype plus RF and anti-CCP, SE, SE plus RF, SE plus anti-CCP, SE plus RF and anti-CCP, genotype plus SE, genotype plus SE plus RF, genotype plus SE plus anti-CCP, and genotype plus SE plus RF and anti-CCP. In addition, other biomarkers as noted above could be used in conjunction with this matrix. One preferred combination is SE and RF. Another preferred combination is genotype plus anti-CCP.

The invention further provides a method of determining the likelihood that a RA patient will show relatively long symptom-free benefit from therapy with a B-cell antagonist. This comprises determining the levels of genotype and/or SE in a genetic sample from the patient, and, optionally, other biomarkers such as seropositivity for RF and/or anti-CCP in a patient sample, wherein the levels of genotype and/or SE, and other optional markers, e.g., anti-CCP and/or RF seropositivity, if assessed, are indicative that a RA patient will show relatively long symptom-free benefit from therapy with a B-cell antagonist.

The invention also provides a method for assessing the response of a RA patient to a B-cell antagonist in vitro by biochemical markers, comprising measuring in a sample the polymorphism of at least PTNP22 R620W SNP or the presence of SE, or both SNP and SE. In a preferred embodiment, at least one additional marker is employed selected from the group consisting of C-reactive protein (CRP), interleukins and other cytokines such as IL-6, serum amyloid A, calcium binding protein S100, osteopontin, anti-CCP, RF, stromelysin 1, collagenase, hyaluronic acid (HA), CD-14, MMP-1, MMP-3, and angiogenesis markers.

In a preferred embodiment the present invention relates to a method for improving the prediction of responsiveness in RA patients versus healthy controls to therapy with a B-cell antagonist by assessing in a sample the polymorphism of at least PTNP22 R620W SNP or the presence of SE, or both SNP and SE. The result is correctly classifying more patients as responsive to the B-cell antagonist as compared to a classification based on anti-CCP or RF alone or in combination.

In another embodiment, the invention relates to a method for determining the sensitivity of a subject with RA to a B-cell antagonist, comprising the steps of obtaining a genetic sample and examining the sample to detect expression of PTNP22 R620W SNP, or SE, or both the SNP and SE, wherein expression of the SNP or SE or both is indicative that the subject is sensitive to the RA-beneficial activity of a B-cell antagonist (such as B-cell depleting activity).

The present invention further provides a method of identifying a biomarker the expression level of which is predictive of the effective responsiveness of a particular patient with RA to a B-cell antagonist. This comprises: (a) measuring the expression level of a candidate biomarker in a panel of cells that displays a range of sensitivities to a B-cell antagonist, and (b) identifying a correlation between the expression level of, seropositivity for, or presence of the candidate biomarker in the cells and the sensitivity of a patient with RA to effective responsiveness to the B-cell antagonist, wherein the correlation indicates that the expression level, seropositivity, or presence of the biomarker is predictive of the responsiveness of the patient to treatment by a B-cell antagonist. In one embodiment of this method the panel of cells is a panel of RA samples prepared from samples derived from patients or experimental animal models. In an additional embodiment the panel of cells is a panel of cell lines in mouse xenografts, wherein responsiveness can, for example, be determined by monitoring a molecular marker of responsiveness, e.g., ACR20. Preferably, the biomarker is genetic and its expression level is analyzed.

The present invention also provides a method of identifying a biomarker that is diagnostic for more effective treatment of RA with a B-cell antagonist comprising: (a) measuring the level of a candidate biomarker in samples from patients with RA, and (b) identifying a correlation between the expression level of, seropositivity for, or presence of the candidate biomarker in the sample from the patient with the effectiveness of treatment of the RA with a B-cell antagonist, wherein the correlation indicates that the biomarker is diagnostic for more effective treatment of the RA with a B-cell antagonist. Preferably, the biomarker is genetic and its expression is analyzed.

In another aspect, the present invention provides a method of identifying a biomarker that is diagnostic for prolonged symptom-free status of a patient with RA when treated with a B-cell antagonist comprising: (a) measuring the level of the candidate biomarker in samples from patients with RA, and (b) identifying a correlation between the expression level, seropositivity, or presence of the candidate biomarker in the sample from the patient with prolonged symptom-free status of that patient when treated with a B-cell antagonist, wherein the correlation of a biomarker with prolonged symptom-free status in the patients indicates the biomarker is diagnostic for prolonged symptom-free status of a patient with RA when treated with a B-cell antagonist.

In all the methods described herein the sample is taken from a patient who is suspected to have, or is diagnosed to have RA, and hence is likely in need of treatment. For assessment of marker expression, patient genetic samples, such as those containing cells, or proteins or nucleic acids produced by these cells, may be used in the methods of the present invention. In the methods of this invention, the level of a genetic biomarker can be determined, e.g., by extracting nucleic acid from the sample and performing a genetic analysis on the nucleic acid such as PCR to determine the genotype and SE expression. Other biomarkers can be assessed by the amount (e.g., absolute amount or concentration) thereof in a sample, preferably in bodily fluids or excretions containing detectable levels of biomarkers.

Bodily fluids or secretions useful as samples (including genetic samples) in the present methods include, e.g., blood, urine, saliva, stool, pleural fluid, lymphatic fluid, sputum, ascites, prostatic fluid, cerebrospinal fluid (CSF), or any other bodily secretion or derivative thereof. The word “blood” is meant to include whole blood, plasma, serum, or any derivative of blood. Assessment of biomarker(s) in bodily fluids or excretions obtained without invasive techniques can sometimes be preferred in circumstances where an invasive sampling method is inappropriate or inconvenient. However, the sample to be tested herein is preferably blood, synovial tissue, or synovial fluid, most preferably blood.

The sample may be frozen, fresh, fixed (e.g., formalin fixed), centrifuged, and/or embedded (e.g., paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation.

Where the genotype (SNP) and/or SE, alone or together with other biomarkers such as, for example, seropositivity for anti-CCP and/or RF, are found to be present in a sample, the patient from whom the sample was procured is concluded to be a candidate for therapy with a B-cell antagonist as disclosed herein. The level of biomarker protein and/or mRNA can be determined using methods well known to those skilled in the art.

Measurement of biomarker expression levels may be performed by using a software program executed by a suitable processor. Suitable software and processors are well known in the art and are commercially available. The program may be embodied in software stored on a tangible medium such as a CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the measurement of the expression levels of the genes identified herein, or their expression products, and the determination that a subject is likely or not likely to respond to treatment with a B-cell antagonist, the assay results, findings, diagnoses, predictions, and/or treatment recommendations are typically recorded and communicated to technicians, physicians, and/or patients, for example. In certain embodiments, computers will be used to communicate such information to interested parties, such as patients and/or the attending physicians. In some embodiments, the assays will be performed or the assay results analyzed in a country or jurisdiction that differs from the country or jurisdiction to which the results or diagnoses are communicated.

In a preferred embodiment, a diagnosis, prediction, and/or treatment recommendation based on the expression level in a test subject of one or more of the biomarkers herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, electronic forms of communication, such as e-mail, or telephone. Communication may be facilitated by use of a computer, such as in the case of e-mail communications. In certain embodiments, the communication containing results of a diagnostic test and/or conclusions drawn from and/or treatment recommendations based on the test may be generated and delivered automatically to the subject using a combination of computer hardware and software that will be familiar to artisans skilled in telecommunications. One example of a healthcare-oriented communications system is described in U.S. Pat. No. 6,283,761; however, the present invention is not limited to methods that utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

Methods for detecting the genetic markers (SE and polymorphism) include protocols that examine the presence and/or expression of the SNP or SE in a sample. Tissue or cell samples from mammals can be conveniently assayed for, e.g., genetic-marker mRNAs or DNAs using Northern-blot, dot-blot, or PCR analysis, array hybridization, RNase protection assay, or DNA SNP chip microarrays, which are commercially available, including DNA microarray snapshots. For example, real-time PCR (RT-PCR) assays such as quantitative PCR assays are well known in the art. In an illustrative embodiment of the invention, a method for detecting a PTPN22 SNP mRNA in a biological sample comprises producing cDNA from the sample by reverse transcription using at least one primer; amplifying the cDNA so produced using PTPN22 SNP polynucleotides as sense and antisense primers to amplify PTPN22 SNP cDNAs therein; and detecting the presence of the amplified PTPN22 SNP cDNA. In addition, such methods can include one or more steps that allow one to determine the levels of PTPN22 SNP mRNA in a biological sample (e.g., by simultaneously examining the levels of a comparative control mRNA sequence of a “housekeeping” gene such as an actin family member). Optionally, the sequence of the amplified PTPN22 SNP cDNA can be determined.

In one specific embodiment, genotyping of the PTPN22 gene 1858C->T polymorphism can be performed by RT-PCR technology, using the TAQMAN™ 5′-allele discrimination assay, a restriction fragment-length polymorphism PCR-based analysis, or a PYROSEQUENCER™ instrument. In addition, the method of detecting a genetic variation or polymorphism set forth in U.S. Pat. No. 7,175,985 may be used. In this method a nucleic acid is synthesized utilizing the hybridized 3′-end, which is synthesized by complementary-strand synthesis, on a specific region of a target nucleotide sequence existing as the nucleotide sequence of the same strand as the origin for the next round of complementary-strand synthesis.

Probes used for PCR may be labeled with a detectable marker, such as, for example, a radioisotope, fluorescent compound, bioluminescent compound, chemiluminescent compound, metal chelator, or enzyme. Such probes and primers can be used to detect the presence of PTPN22 SNP or SE polynucleotides in a sample and as a means for detecting a cell expressing SE or PTPN22 SNP proteins. As will be understood by the skilled artisan, a great many different primers and probes may be prepared based on the sequences provided herein and used effectively to amplify, clone, and/or determine the presence and/or levels of PTPN22 SNP or SE mRNAs.

Other methods include protocols that examine or detect mRNAs, such as PTPN22 SNP mRNAs, in a tissue or cell sample by microarray technologies. With the use of nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment (see, e.g., WO 2001/75166). See, for example, U.S. Pat. No. 5,700,637, U.S. Pat. No. 5,445,934, and U.S. Pat. No. 5,807,522; Lockart, Nature Biotechnology, 14:1675-1680 (1996); and Cheung et al., Nature Genetics, 21(Suppl): 15-19 (1999) for a discussion of array fabrication.

In addition, the DNA profiling and SNP detection method utilizing microarrays described in EP 1753878 may be employed. This method rapidly identifies and distinguishes between different DNA sequences utilizing short tandem repeat (STR) analysis and DNA microarrays. In one embodiment, a labeled STR target sequence is hybridized to a DNA microarray carrying complementary probes. These probes vary in length to cover the range of possible STRs. The labeled single-stranded regions of the DNA hybrids are selectively removed from the microarray surface utilizing a post-hybridization enzymatic digestion. The number of repeats in the unknown target is deduced based on the pattern of target DNA that remains hybridized to the microarray.

One example of a microarray processor is the Affymetrix GENECHIP® system, which is commercially available and comprises arrays fabricated by direct synthesis of oligonucleotides on a glass surface. Other systems may be used as known to one skilled in the art.

Other methods for determining the level of the biomarker besides RT-PCR or another PCR-based method include proteomics techniques, as well as individualized genetic profiles that are necessary to treat RA based on patient response at a molecular level. The specialized microarrays herein, e.g., oligonucleotide microarrays or cDNA microarrays, may comprise one or more biomarkers having expression profiles that correlate with either sensitivity or resistance to one or more anti-CD20 antibodies. Additionally, SNPs can be detected using electronic circuitry on silicon microchips, as disclosed, for example, in WO 2000/058522.

Identification of biomarkers that provide rapid and accessible readouts of efficacy, drug exposure, or clinical response is increasingly important in the clinical development of drug candidates. Embodiments of the invention include measuring changes in the levels of secreted proteins, or plasma biomarkers, which represent one category of biomarker. In one aspect, plasma samples, which represent a readily accessible source of material, serve as surrogate tissue for biomarker analysis.

Many references are available to provide guidance in applying the above techniques: Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-158 (CRC Press, Inc., 1987). Northern-blot analysis is a conventional technique well known in the art and described, for example, in Molecular Cloning, a Laboratory Manual, 2nd edition, Sambrook et al. (Cold Spring Harbor Press, NY, 1989). Typical protocols for evaluating the status of genes and gene products are found, for example in Ausubel et al. eds., Current Protocols In Molecular Biology (1995), Units 2 (Northern Blotting), 4 (Southern Blotting), (Immunoblotting) and 18 (PCR Analysis).

As to detection of protein biomarkers such as anti-CCP and RF antibodies, e.g., various protein assays are available. For example, the sample may be contacted with an antibody specific for the biomarker under conditions sufficient for an antibody-biomarker complex to form, and then the complex is detected. The presence of the protein biomarker may be assessed in a number of ways, such as by Western blotting (with or without immunoprecipitation), two-dimensional SDS-PAGE, immunoprecipitation, fluorescence-activated cell sorting (FACS), flow cytometry, and ELISA procedures for assaying a wide variety of tissues and samples, including plasma or serum. A wide range of immunoassay techniques using such an assay format are available; see, e.g., U.S. Pat. No. 4,016,043, U.S. Pat. No. 4,424,279, and U.S. Pat. No. 4,018,653. These include both single-site and two-site or “sandwich” assays of the non-competitive types as well as the traditional competitive binding assays. These assays also include direct binding of a labeled antibody to a target biomarker.

Sandwich assays are among the most useful and commonly used assays. A number of variations of the sandwich assay technique exist, and all are encompassed by this invention. Briefly, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal, is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well known in the art and generally consist of cross-linking, covalently binding, or physically adsorbing, and the polymer-antibody complex is washed in preparation for the test sample. An aliquot of the sample to be tested is then added to the solid-phase complex and incubated for a period of time sufficient (e.g., 2-40 minutes or overnight if more convenient) and under suitable conditions (e.g., from room temperature to 40° C., such as between 25° C. and 32° C. inclusive) to allow binding of any subunit present in the antibody. Following the incubation period, the antibody subunit solid phase is washed, dried, and incubated with a second antibody specific for a portion of the biomarker. The second antibody is linked to a reporter molecule that is used to indicate the binding of the second antibody to the molecular marker.

An alternative method involves immobilizing the target biomarkers in the sample and then exposing the immobilized target to specific antibody that may or may not be labeled with a reporter molecule. Depending on the amount of target and the strength of the reporter molecule signal, a bound target may be detectable by direct labeling with the antibody. Alternatively, a second labeled antibody, specific to the first antibody, is exposed to the target-first antibody complex to form a target-first antibody-second antibody tertiary complex. The complex is detected by the signal emitted by the reporter molecule. By “reporter molecule,” as used in the present specification, is meant a molecule that, by its chemical nature, provides an analytically identifiable signal that allows the detection of antigen-bound antibody. The most commonly used reporter molecules in this type of assay are either enzymes, fluorophores, radionuclide-containing molecules (i.e., radioisotopes), or chemiluminescent molecules.

In the case of an enzyme immunoassay (EIA), an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker that was present in the sample. Alternatively, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state of excitability in the molecule, followed by emission of the light at a characteristic color visually detectable with a light microscope. As in the EIA, the fluorescent-labeled antibody is allowed to bind to the first antibody-molecular marker complex. After washing off the unbound reagent, the remaining tertiary complex is then exposed to the light of the appropriate wavelength; the fluorescence observed indicates the presence of the molecular marker of interest. Immunofluorescence and EIA techniques are both very well established in the art. However, other reporter molecules, such as radioisotope, chemiluminescent, or bioluminescent molecules, may also be employed.

Anti-CCP antibodies, in particular, can be analyzed by an EIA and serological assay, including a second-generation ELISA (IMMUNOSCAN RA™), as well as an agglutination assay (Latex and Waaler-Rose) and specific ELISA (IgM, IgG and IgA). For example, the presence of anti-CCP in sera may be measured using anti-CCP-ELISA (CCP1 test, cf. Schellekens et al., Arthr. Rheum, 43:155-163 (2000)). Commercially available ELISAs can be used, including IMMUNOSCAN RA™ (Eurodiagnostica, The Netherlands), Inova Diagnostics and Axis-Shield Diagnostics. Detection can be made using synthetic citrullinated peptide variants. Anti-CCP2 concentrations can be measured using a second-generation ELISA. A third-generation ELISA for anti-CCP, marketed by Inova Diagnostics, may also be used. Associations between anti-CCP antibodies and clinical and laboratory parameters can be determined by Fisher's exact test. Anti-CCP can also be measured as described by van Venroij et al. in WO 03/050542. The assay may be set up by using one or more CCPs as antigen and detecting the binding of anti-CCP antibodies comprised in a sample to the CCP antigen by appropriate means. Anti-CCP antibodies may additionally be detected by homogeneous assays formats, e.g., by agglutination of latex particles coated with CCP. Also, a heterogeneous immunoassay may be used to measure anti-CCP. Such heterogeneous measurement is based on directly or indirectly coating CCP to a solid phase, incubating the solid phase with a sample known or suspected to comprise anti-CCP antibodies under conditions allowing for binding of anti-CCP antibodies to CCP, and directly or indirectly detecting the anti-CCP antibody bound. A further assay format is the so-called double-antigen bridge assay, wherein in case of an anti-CCP measurement, CCPs are used both at the solid-phase side as well as at the detection side of this immunoassay.

Abreu et al., “Multiplexed immunoassay for detection of rheumatoid factors by FIDIS technology,” Annals of the New York Academy of Sciences, 1050(Autoimmunity):357-363 (2005) compares FIDIS RHEUMA™, a multiplexed immunoassay designed for simultaneous detection of IgM class RF directed against Fc determinants of IgG from humans and animals, with agglutination and ELISA and evaluates the clinical sensitivity and specificity of biological markers for RA. FIDIS technology was employed using the LUMINEX™ system and consisted of distinct color-coded microsphere sets, a flow cytometer, and digital signal processing hardware and software. Agglutination and ELISA tests can be performed with commercial kits. For human specificity, FIDIS may be used as an alternative to latex agglutination or ELISA. For animal specificity, FIDIS may be used as an alternative to WAALER-ROSE™ technology and ELISA. Detection of IgG anti-CCP by ELISA using immunofluorescence is also an embodiment herein. Dubois-Galopin et al., “Evaluation of a new fluorometric immunoassay for the detection of anti-cyclic citrullinated peptide autoantibodies in rheumatoid arthritis,” Annales de Biologie Clinique, 64(2):162-165 (2006) evaluated the measurement of anti-CCP antibodies by a new fluorescent EIA, called EliA CCP, fully automated onto UNICAP100ε™, This compares well with an ELISA method (Euroimmun) and is also useful herein.

RFs can be analyzed by, for example, latex-enhanced turbidimetry or latex agglutination and two isotype-specific (IgM and IgA) EIAs that are commercially available, or ELISAs. Isotypes of anti-CCPs can be detected by similar means.

II. Statistics

As used herein, the general form of a prediction rule consists in the specification of a function of one or multiple biomarkers potentially including clinical covariates to predict response or non-response, or more generally, predict benefit or lack of benefit in terms of suitably defined clinical endpoints.

The simplest form of a prediction rule consists of a univariate model without covariates, wherein the prediction is determined by means of a cutoff or threshold. This can be phrased in terms of the Heaviside function for a specific cutoff c and a biomarker measurement x, where the binary prediction A or B is to be made, then

If H(x−c)=0, then predict A.

If H(x−c)=1, then predict B.

This is the simplest way of using univariate biomarker measurements in prediction rules. If such a simple rule is sufficient, it allows for a simple identification of the direction of the effect, i.e., whether high or low expression levels are beneficial for the patient.

The situation can be more complicated if clinical covariates need to be considered and/or if multiple biomarkers are used in multivariate prediction rules. The two hypothetical examples below illustrate the issues involved:

### Covariate Adjustment (Hypothetical Example):

For a biomarker X it is found in a clinical trial population that high expression levels are associated with a worse clinical response (univariate analysis). A closer analysis shows that there are two types of RA clinical responses in the population, one of which possesses a worse response than the other one and at the same time the biomarker expression for this overall RA group is generally higher. An adjusted covariate analysis reveals that for each of the RA types the relation of clinical benefit and clinical response is reversed, i.e., within the RA types, lower expression levels are associated with better clinical response. The overall opposite effect was masked by the covariate RA type—and the covariate-adjusted analysis as part of the prediction rule reversed the direction.

### Multivariate Prediction (Hypothetical Example):

For a biomarker X it is found in a clinical trial population that high expression levels are slightly associated with a worse clinical response (univariate analysis). For a second biomarker Y a similar observation was made by univariate analysis. The combination of X and Y revealed that a good clinical response is seen if both biomarkers are low. This makes the rule to predict benefit if both biomarkers are below some cutoffs (AND-connection of a Heaviside prediction function). For the combination rule, a simple rule no longer applies in a univariate sense; for example, having low expression levels in X will not automatically predict a better clinical response.

These simple examples show that prediction rules with and without covariates cannot be judged on the univariate level of each biomarker. The combination of multiple biomarkers plus a potential adjustment by covariates does not allow assigning simple relationships to single biomarkers. Since the marker genes, particularly in serum, may be used in multiple-marker prediction models potentially including other clinical covariates, the direction of a beneficial effect of a single marker gene within such models cannot be determined in a simple way, and may contradict the direction found in univariate analyses, i.e., the situation as described for the single-marker gene.

III. Treatment with Antagonist

The present invention provides a method of treating RA in a patient comprising administering an effective amount of a B-cell antagonist to the patient to treat the RA, provided that a PTPN22 R620W SNP or SE or both SNP and SE are present in a genetic sample from the patient.

The invention also supplies a method of treating RA in a patient comprising administering to the patient an effective amount of a B-cell antagonist, wherein before the administration, expression of PTNP22 R620W SNP, or SE, or both the SNP and SE was detected in a genetic sample from the patient.

The invention additionally provides a method of treating RA in a patient comprising administering to the patient an effective amount of a B-cell antagonist, wherein before the administration a genetic sample from the patient was determined to exhibit expression of PTNP22 R620W SNP, or SE, or both the SNP and SE, whereby the expression indicates that the patient will respond to treatment with the antagonist.

The invention also affords a method of treating RA in a patient comprising administering to the patient an effective amount of a B-cell antagonist, wherein before the administration a genetic sample from the patient was determined to exhibit expression of PTNP22 R620W SNP, or SE, or both the SNP and SE, whereby the expression indicates that the patient is likely to respond favorably to treatment with the antagonist.

In one preferred embodiment, expression of the SNP is assessed, but not SE. In another preferred embodiment, expression of the SE is assessed, but not the SNP. In a third preferred embodiment, expression of both the SNP and SE is assessed.

In another aspect, the expression of the SNP or SE or both is assessed not in combination with another biomarker. In another, more preferred aspect, the expression of the SNP or SE or both is assessed in combination with another biomarker, preferably assessed for seropositivity for one or both of the additional biomarkers anti-CCP antibody and RF in a sample from the patient. Seropositivity for one or both of these additional biomarkers would indicate that the RA will respond effectively to treatment with the B-cell antagonist, such as anti-CD20 or anti-CD22 antibody. In such method, the additional biomarker is anti-CCP antibody, preferably of the IgG or IgM isotype, or the additional biomarker is a RF, preferably having an IgA, IgG, or IgM isotype. In another aspect, the additional biomarkers are both anti-CCP antibody and RF.

In a particularly preferred aspect, expression of SE is assessed along with seropositivity for RF, without assessment of the SNP or anti-CCP antibody, i.e., the SE is present along with seropositivity for RF, without the presence of the SNP or anti-CCP antibody. In another especially preferred aspect, the SNP is present along with seropositivity for anti-CCP antibody, without presence of the SE or RF.

The effectiveness of treatment in the preceding methods can, for example, be determined by using the ACR and/or EULAR clinical response parameters in the patients with RA, or by assaying a molecular determinant of the degree of RA in the patient. Thus, for example, a clinician may use any of several methods known in the art to measure the effectiveness of a particular dosage scheme of a B-cell antagonist. For example, x-ray technology can be used to determine the extent of joint destruction and damage in the patient, and the scale of ACR20, ACR50, and ACR70 can be used to determine relative effective responsiveness to the therapy. Dosage regimens may be adjusted to provide the optimum desired response (e.g., a therapeutic response). For example, a dose may be administered, several divided doses may be administered over time, or the dose may be proportionally reduced or increased as indicated by exigencies of the therapeutic situation.

Once the patient population most responsive to treatment with the antagonist has been identified, treatment with the antagonist herein, alone or in combination with other medicaments, results in an improvement in the RA or joint damage, including signs or symptoms thereof. For instance, such treatment may result in an improvement in ACR measurements relative to a patient treated with the second medicament only (e.g., an immunosuppressive agent such as MTX), and/or may result in an objective response (partial or complete, preferably complete) as measured by ACR. Moreover, treatment with the combination of an antagonist herein and at least one second medicament preferably results in an additive, more preferably synergistic (or greater than additive) therapeutic benefit to the patient. Preferably, in this method the timing between at least one administration of the second medicament and at least one administration of the antagonist herein is about one month or less, more preferably, about two weeks or less.

It will be appreciated by one of skill in the medical arts that the exact manner of administering to the patient a therapeutically effective amount of a B-cell antagonist following a diagnosis of a patient's likely responsiveness to the antagonist will be at the discretion of the attending physician. The mode of administration, including dosage, combination with other anti-RA agents, timing and frequency of administration, and the like, may be affected by the extent of the diagnosis of the patient's likely responsiveness to such antagonist (for example, higher seropositivity of anti-CCP or RF than normal), as well as the patient's condition and history.

The composition comprising an antagonist will be formulated, dosed, and administered in a fashion consistent with good medical practice. Factors for consideration in this context include the particular type of RA being treated, the particular mammal being treated, the clinical condition of the individual patient, the cause of the RA, the site of delivery of the antagonist, possible side-effects, the type of antagonist, the method of administration, the scheduling of administration, and other factors known to medical practitioners. The effective amount of the antagonist to be administered will be governed by such considerations.

A physician having ordinary skill in the art can readily determine and prescribe the effective amount of the pharmaceutical composition required, depending on such factors as the particular antagonist type and safety profile. For example, the physician could start with doses of such antagonist, such as an anti-CD20 or anti-CD22 antibody or immunoadhesin, employed in the pharmaceutical composition at levels lower than that required to achieve the desired therapeutic effect to assess safety, and gradually increase the dosage until the desired effect (without compromising safety) is achieved. The effectiveness of a given dose or treatment regimen of the antagonist can be determined, for example, by assessing signs and symptoms in the patient using the standard RA measures of efficacy.

As a general proposition, the effective amount of the antagonist administered parenterally per dose will be in the range of about 20 mg to about 5000 mg, by one or more dosages. Exemplary dosage regimens for intact antibodies such as anti-CD20 antibodies and anti-CD22 antibodies, and BAFF and APRIL antagonists, include 375 mg/m² weekly×4 (e.g., on days 1, 8, 15, and 22); or 500 mg×2 (e.g., on days 1 and 15), or 1000 mg×2 (e.g., on days 1 and 15); or 1 gram×3 (e.g., on days 1, 15, and 21); or 200 mg×1-4; or 300 mg×1-4, or 400 mg×1-4; or 500 mg×3-4; or 1 gram×4.

Preferably, the antagonist is administered in a dose of about 0.2 to 4 grams, more preferably about 0.2 to 3.5 grams, more preferably about 0.4 to 2.5 grams, more preferably about 0.5 to 1.5 grams, and even more preferably about 0.7 to 1.1 gram. More preferably, such doses apply to antagonists that are antibodies or immunoadhesins.

Alternatively, the antagonist is anti-CD20 antibody administered at a dose of about 1000 mg×2 on days 1 and 15 intravenously at the start of the treatment. In another alternative preferred embodiment, the anti-CD20 antibody is administered as a single dose or as two infusions, with each dose at about 200 mg to 1.2 g, more preferably about 200 mg to 1.1 g, and still more preferably about 200 mg to 900 mg.

In a preferred aspect, the antagonist is administered at a frequency of one to four doses within a period of about one month. The antagonist is preferably administered in two to three doses. In addition, the antagonist is preferably administered within a period of about two to three weeks.

As noted above, however, these suggested amounts of antagonist and frequency of dosing are subject to a great deal of therapeutic discretion. The key factor in selecting an appropriate dose and schedule is the result obtained, as indicated above. For example, relatively higher doses may be needed initially for the treatment of ongoing and acute RA. To obtain the most efficacious results, once antagonist therapy is predicted by the biomarkers herein the antagonist is administered as close to the first sign, diagnosis, appearance, or occurrence of the RA as possible or during remissions of the RA.

In all the inventive methods set forth herein, the antagonist (such as an antibody that binds to a B-cell surface marker) may be unconjugated, such as a naked antibody, or may be conjugated with another molecule for further effectiveness, such as, for example, to improve half-life. The most preferred antagonist is a CD20, CD22, CD23, CD40, or BAFF antagonist, more preferably antibodies or immunoadhesins such as a BR3-Fc or TACI-Ig fusion molecule (same as TACI-Ig or atacicept available from ZymoGenetics; see also Gross et al., Immunity, 15:289-291 (2001) and US 2007/0071760).

The preferred antagonist antibody herein is a chimeric, humanized, or human antibody, more preferably, an anti-CD20, anti-CD22, or anti-BR3 antibody, and most preferably rituximab, epratuzumab, a 2H7 antibody (including one that comprises the L-chain variable region sequence of SEQ ID NO:1 and the H-chain variable region sequence of SEQ ID NO:2, one that comprises the L-chain variable region sequence of SEQ ID NO:3 and the H-chain variable region sequence of SEQ ID NO:4, one that comprises the L-chain variable region sequence of SEQ ID NO:3 and the H-chain variable region sequence of SEQ ID NO:5, one that comprises the full-length L chain of SEQ ID NO:6 and the full-length H chain of SEQ ID NO:7, one that comprises the full-length L chain of SEQ ID NO:6 and the full-length H chain of SEQ ID NO:8, one that comprises the full-length L chain of SEQ ID NO:9 and the full-length H chain of SEQ ID NO:10, one that comprises the full-length L chain of SEQ ID NO:9 and the full-length H chain of SEQ ID NO:11, one that comprises the full-length L chain of SEQ ID NO:9 and the full-length H chain of SEQ ID NO:12, one that comprises the full-length L chain of SEQ ID NO:9 and the full-length H chain of SEQ ID NO:13, one that comprises the full-length L chain of SEQ ID NO:9 and the full-length H chain of SEQ ID NO:14, or one that comprises the full-length L chain of SEQ ID NO:6 and the full-length H chain of SEQ ID NO:15), chimeric or humanized A20 antibody (Immunomedics), HUMAX-CD20™ human anti-CD20 antibody (Genmab), single-chain proteins binding to CD20 (a small modular immunopharmaceutical (SMIP™) drug candidate (e.g., TRU-015; Trubion Pharm Inc.; Wyeth), an AME antibody against CD20 (Lilly) such as those set forth above (e.g., AME-33, AME-133, or AME-133v), or a humanized type II CD20 IgG1 antibody called GA101 (GlyArt Biotechnology AG; Roche) (see, e.g., US 2005/0123546). Still more preferred is an anti-CD20 antibody selected from the group consisting of rituximab, HUMAX-CD20™, epratuzumab, TRU-015, GA101, or a 2H7 antibody, such as those set forth above.

In a further embodiment of the methods herein, the subject has never been previously treated with one or more drugs, such as with a TNF-α inhibitor, e.g., TNFR-Ig or an anti-TNF-α or anti-TNF-α receptor antibody, to treat, for example, RA, or with immunosuppressive agent(s) to treat joint damage or an underlying cause such as an autoimmune disorder, and/or has never been previously treated with a B-cell antagonist (e.g., antibody to a B-cell surface marker such as an anti-CD20, anti-CD22, or anti-BR3 antibody). In another embodiment, the subject has never been previously treated with an integrin antagonist such as anti-α4 integrin antibody or co-stimulation modulator, an immunosuppressive agent, a cytokine antagonist, an anti-inflammatory agent such as a NSAID, a DMARD other than MTX, except for azathioprine and/or leflunomide, a cell-depleting therapy, including investigational agents (e.g., CAMPATH, anti-CD4, anti-CD5, anti-CD3, anti-CD19, anti-CD11a, anti-CD22, or BLys/BAFF), a live/attenuated vaccine within 28 days prior to baseline, or a corticosteroid such as an intra-articular or parenteral glucocorticoid within 4 weeks prior to baseline. More preferably, the subject has never been treated with an immunosuppressive agent, cytokine antagonist, integrin antagonist, corticosteroid, analgesic, a DMARD, or a NSAID. Still more preferably, the subject has never been treated with an immunosuppressive agent, cytokine antagonist, integrin antagonist, corticosteroid, DMARD, or NSAID.

In a further aspect, the subject may have had a relapse with the RA or joint damage or suffered organ damage such as kidney damage before being treated in any of the methods above, including after the initial or a later antagonist or antibody exposure. However, preferably, the subject has not relapsed with the RA or joint damage and more preferably has not had such a relapse before at least the initial treatment.

In a further embodiment, the subject does not have a malignancy, including a B-cell malignancy, solid tumors, hematologic malignancies, or carcinoma in situ (except basal cell and squamous cell carcinoma of the skin that have been excised and cured). In a still further embodiment, the subject does not have rheumatic autoimmune disease other than RA, or significant systemic involvement secondary to RA (including but not limited to vasculitis, pulmonary fibrosis, or Felty's syndrome). In another embodiment, the subject does have secondary Sjögren's syndrome or secondary limited cutaneous vasculitis. In another embodiment, the subject does not have functional class IV as defined by the ACR Classification of Functional Status in RA. In a further embodiment, the subject does not have inflammatory joint disease other than RA (including, but not limited to, gout, reactive arthritis, psoriatic arthritis, seronegative spondyloarthropathy, or Lyme disease), or other systemic autoimmune disorder (including, but not limited to, SLE, inflammatory bowel disease, scleroderma, inflammatory myopathy, mixed connective tissue disease, or any overlap syndrome). In another embodiment, the subject does not have juvenile idiopathic arthritis (JIA), juvenile RA (JRA), and/or RA before age 16. In another embodiment, the subject does not have significant and/or uncontrolled cardiac or pulmonary disease (including obstructive pulmonary disease), or significant concomitant disease, including but not limited to, nervous system, renal, hepatic, endocrine or gastrointestinal disorders, nor primary or secondary immunodeficiency (history of, or currently active), including known history of HIV infection. In another aspect, the subject does not have any neurological (congenital or acquired), vascular or systemic disorder that could affect any of the efficacy assessments, in particular, joint pain and swelling (e.g., Parkinson's disease, cerebral palsy, or diabetic neuropathy). In a still further embodiment, the subject does not have MS. In a yet further aspect, the subject does not have lupus or Sjögren's syndrome. In still another aspect, the subject does not have an autoimmune disease other than RA. In yet another aspect of the invention, any joint damage in the subject is not associated with an autoimmune disease or with an autoimmune disease other than RA, or with a risk of developing an autoimmune disease or an autoimmune disease other than RA.

For purposes of these lattermost statements, an “autoimmune disease” herein is a disease or disorder arising from and directed against an individual's own tissues or organs or a co-segregate or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. Without being limited to any one theory regarding B-cell mediated autoimmune disease, it is believed that B cells demonstrate a pathogenic effect in human autoimmune diseases through a multitude of mechanistic pathways, including autoantibody production, immune complex formation, dendritic and T-cell activation, cytokine synthesis, direct chemokine release, and providing a nidus for ectopic neo-lymphogenesis. Each of these pathways may participate to different degrees in the pathology of autoimmune diseases. “Autoimmune disease” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease that can affect multiple organ systems (for example, SLE, RA, polymyositis, etc.). Preferred such diseases include autoimmune rheumatologic disorders (such as, for example, RA, Sjögren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, MS, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, RA, ulcerative colitis, ANCA-associated vasculitis, lupus, MS, Sjögren's syndrome, Graves' disease, IDDM, pernicious anemia, thyroiditis, and glomerulonephritis.

In another preferred aspect of the above-described method, the subject was administered MTX prior to the baseline or start of treatment. More preferably, the MTX was administered at a dose of about 10-25 mg/week. Also, preferably, the MTX was administered for at least about 12 weeks prior to the baseline, and still more preferably the MTX was administered at a stable dose the last four weeks prior to the baseline. In other embodiments, the MTX was administered perorally or parenterally.

In a particularly preferred embodiment of the above-identified methods, the subject has exhibited an inadequate response to one or more TNF-α inhibitors or to MTX. In another aspect, the subject has been refractory to a B-cell antagonist, such as those other than rituximab or a 2H7 antibody. However, the subject may also have been refractory to rituximab or a 2H7 antibody.

In another preferred aspect, MTX is administered to the subject along with the antagonist, for example, anti-CD20 antibody. In another aspect, the antagonist is an anti-CD20 antibody that is administered at a dose of about 1000 mg×2 on days 1 and 15 intravenously at the start of the treatment or is administered a dose of about 400 to 800 mg as a single dose or as two doses, such as infusions.

Also included herein, after the diagnosis step, is a method of monitoring the treatment of bone or soft tissue joint damage in a subject comprising administering an effective amount of a B-cell antagonist (such as an antibody thereto, including an anti-CD20, anti-CD22, or anti-BR3 antibody) to the subject and measuring by imaging techniques such as MRI or radiography after at least about three months, preferably about 24 weeks, from the administration whether the bone or soft tissue joint damage has been reduced over baseline prior to the administration, wherein a decrease versus baseline in the subject after treatment indicates the antagonist such as an anti-CD20, anti-CD22, or anti-BR3 antibody is having an effect on the joint damage. Preferably, the degree of reduction versus baseline is measured a second time after the administration of the antagonist such as an antibody or immunoadhesin.

In yet another aspect, the invention provides, after the diagnosis step, a method of determining whether to continue administering a B-cell antagonist (such as an antibody thereto or immunoadhesin, including an anti-CD20 antibody) to a subject with bone or soft tissue joint damage comprising measuring reduction in joint damage in the subject, using imaging techniques, such as radiography and/or MRI, after administration of the antagonist a first time, measuring reduction in joint damage in the subject, using imaging techniques such as radiography and/or MRI after administration of the antagonist a second time, comparing imaging findings in the subject at the first time and at the second time, and if the score is less at the second time than at the first time, continuing administration of the antagonist.

In a still further embodiment, a step is included in the treatment method to test for the subject's response to treatment after the administration step to determine that the level of response is effective to treat the bone or soft tissue joint damage. For example, a step is included to test the imaging (radiographic and/or MRI) score after administration and compare it to baseline imaging results obtained before administration to determine if treatment is effective by measuring if, and by how much, it has been changed. This test may be repeated at various scheduled or unscheduled time intervals after the administration to determine maintenance of any partial or complete remission. Alternatively, the methods herein comprise a step of testing the subject, before administration, to see if one or more biomarkers or symptoms are present for joint damage, as set forth above. In another method, a step may be included to check the subject's clinical history, as detailed above, for example, to rule out infections or malignancy as causes, for example, primary causes, of the subject's condition, prior to administering the antagonist to the subject. Preferably, the joint damage is primary (i.e., the leading disease), and is not secondary, such as secondary to infection or malignancy, whether solid or liquid tumors.

In one embodiment of all the methods herein, the antagonist (for example, anti-CD 20 antibody) is the only medicament administered to the subject to treat the RA, i.e., no other medicament than the antagonist is administered to the subject to treat the RA.

In any of the methods herein, preferably the antagonist is one of the medicaments used to treat the RA. Thus, one may administer to the subject along with the B-cell antagonist an effective amount of a second medicament (where the B-cell antagonist (e.g., an anti-CD20 antibody or BR3-Fc) is a first medicament). The second medicament may be one or more medicaments, and includes, for example, an immunosuppressive agent, a cytokine antagonist such as a cytokine antibody, an integrin antagonist (e.g., antibody), a corticosteroid, or any combination thereof. The type of such second medicament depends on various factors, including the type of RA and/or joint damage, the severity of the RA and/or joint damage, the condition and age of the subject, the type and dose of the first medicament employed, etc.

Examples of such additional medicaments include an immunosuppressive agent (such as mitoxantrone (NOVANTRONE®), MTX, cyclophosphamide, chlorambucil, leflunomide, and azathioprine), intravenous immunoglobulin (gamma globulin), lymphocyte-depleting therapy (e.g., mitoxantrone, cyclophosphamide, CAMPATH™ antibodies, anti-CD4, cladribine, a polypeptide construct with at least two domains comprising a de-immunized, autoreactive antigen or its fragment that is specifically recognized by the Ig receptors, of autoreactive B-cells (WO 2003/68822), total body irradiation, and bone marrow transplantation), integrin antagonist or antibody (e.g., an LFA-1 antibody such as efalizumab/RAPTIVA® commercially available from Genentech, or an alpha 4 integrin antibody such as natalizumab/ANTEGREN® available from Biogen, or others as noted above), drugs that treat symptoms secondary or related to RA and/or joint damage such as those noted herein, steroids such as corticosteroid (e.g., prednisolone, methylprednisolone such as SOLU-MEDROL™ methylprednisolone sodium succinate for injection, prednisone such as low-dose prednisone, dexamethasone, or glucocorticoid, e.g., via joint injection, including systemic corticosteroid therapy), non-lymphocyte-depleting immunosuppressive therapy (e.g., MMF or cyclosporine), a TNF-α inhibitor such as an antibody to TNF-α or its receptor or TNFR-Ig (e.g., etanercept), DMARD, NSAID, plasmapheresis or plasma exchange, trimethoprim-sulfamethoxazole (BACTRIM™, SEPTRA™), MMF, H2-blockers or proton-pump inhibitors (during the use of potentially ulcerogenic immunosuppressive therapy), levothyroxine, cyclosporin A (e.g., SANDIMMUNE®), somatostatin analogue, a DMARD or NSAID, cytokine antagonist such as antibody, anti-metabolite, immunosuppressive agent, rehabilitative surgery, radioiodine, thyroidectomy, anti-IL-6 receptor antagonist/antibody (e.g., ACTEMRA™ (tocilizumab)), or another B-cell antagonist such as BR3-Fc, TACI-Ig, anti-BR3 antibody, anti-CD40 receptor or anti-CD40 ligand (CD154), agent blocking CD40-CD40 ligand, epratuzumab (anti-CD22 antibody), lumiliximab (anti-CD23 antibody), or anti-CD20 antibody such as rituximab or 2H7 antibody.

Preferred such medicaments include gamma globulin, an integrin antagonist, anti-CD 4, cladribine, trimethoprimsulfamethoxazole, an H2-blocker, proton-pump inhibitor, cyclosporine, TNF-α inhibitor, DMARD, NSAID (to treat, for example, musculoskeletal symptoms), levothyroxine, cytokine antagonist (including cytokine-receptor antagonist), anti-metabolite, immunosuppressive agent such as MTX or a corticosteroid, bisphosphonate, and another B-cell antagonist, such as an anti-CD20 antibody, anti-CD22 antibody, anti-BR 3 antibody, lumiliximab (anti-CD23 antibody), BR3-Fc, or TACI-Ig.

The more preferred such medicaments are an immunosuppressive agent such as MTX or a corticosteroid, a DMARD, an integrin antagonist, a NSAID, a cytokine antagonist, a bisphosphonate, or a combination thereof.

In one particularly preferred embodiment, the second medicament is a DMARD, which is preferably selected from the group consisting of auranofin, chloroquine, D-penicillamine, injectable gold, oral gold, hydroxychloroquine, sulfasalazine, myocrisin, and MTX.

In another such embodiment, the second medicament is a NSAID, which is preferably selected from the group consisting of: fenbufen, naprosyn, diclofenac, etodolac and indomethacin, aspirin, and ibuprofen.

In a further such embodiment, the second medicament is an immunosuppressive agent, which is preferably selected from the group consisting of etanercept, infliximab, adalimumab, leflunomide, anakinra, azathioprine, MTX, and cyclophosphamide.

In other preferred aspects, the second medicament is selected from the group consisting of anti-α4, etanercept, infliximab, etanercept, adalimumab, kinaret, efalizumab, OPG, RANK-Fc, anti-RANKL, pamidronate, alendronate, actonel, zolendronate, clodronate, MTX, azulfidine, hydroxychloroquine, doxycycline, leflunomide, SSZ, prednisolone, IL-1 receptor antagonist, prednisone, and methylprednisolone.

In still preferred embodiments, the second medicament is selected from the group consisting of infliximab, an infliximab/MTX combination, etanercept, a corticosteroid, cyclosporin A, azathioprine, auranofin, hydroxychloroquine (HCQ), a combination of prednisolone, MTX, and SSZ, a combination of MTX, SSZ, and HCQ, a combination of cyclophosphamide, azathioprine, and HCQ, and a combination of adalimumab with MTX. If the second medicament is a corticosteroid, preferably it is prednisone, prednisolone, methylprednisolone, hydrocortisone, or dexamethasone. Also, preferably, the corticosteroid is administered in lower amounts than are used if the antagonist is not administered to a subject treated with a corticosteroid as standard-of-care therapy. Most preferably, the second medicament is MTX.

All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The antagonist herein is administered by any suitable means, including parenteral, topical, intraperitoneal, intrapulmonary, intranasal, and/or intralesional administration. Parenteral infusions include intramuscular, intravenous (i.v.), intraarterial, intraperitoneal, or subcutaneous (s.c.) administration. Intrathecal administration is also suitable (see, e.g., US 2002/0009444, Grillo-Lopez, concerning intrathecal delivery of an anti-CD20 antibody). Also the antagonist may suitably be administered by pulse infusion, e.g., with declining doses of the antagonist. Preferably if the antagonist is an antibody or immunoadhesin, the dosing is given by i.v. or s.c. means, and more preferably by i.v. infusion(s) or injection(s).

In one embodiment, the antagonist such as an anti-CD20 antibody is administered as a slow i.v. infusion rather than an i.v. push or bolus. For example, in one aspect a steroid such as prednisolone or methyl-prednisolone (e.g., about 80-120 mg i.v., more specifically about 100 mg i.v.) is administered about 30 minutes prior to any infusion of an anti-CD20 antibody. The anti-CD20 antibody is, for example, infused through a dedicated line.

For the initial dose of a multi-dose exposure to anti-CD20 antibody, or for the single dose if the exposure involves only one dose, such infusion is preferably commenced at a rate of about 50 mg/hour. This may be escalated, e.g., at a rate of about 50 mg/hour increments every about 30 minutes to a maximum of about 400 mg/hour. However, if the subject is experiencing an infusion-related reaction, the infusion rate is preferably reduced, e.g., to half the current rate, e.g., from 100 mg/hour to 50 mg/hour. Preferably, the infusion of such dose of anti-CD20 antibody (e.g., an about 1000-mg total dose) is completed at about 255 minutes (4 hours 15 min.). Optionally, the subjects receive a prophylactic treatment of acetaminophen/paracetamol (e.g., about 1 g) and diphenhydramine HCl (e.g., about 50 mg or equivalent dose of similar agent) by mouth about 30 to 60 minutes prior to the start of an infusion.

If more than one infusion (dose) of anti-CD20 antibody is given to achieve the total exposure, the second or subsequent anti-CD20 antibody infusions in this embodiment are preferably commenced at a higher rate than the initial infusion, e.g., at about 100 mg/hour. This rate may be escalated, e.g., at a rate of about 100 mg/hour increments every about 30 minutes to a maximum of about 400 mg/hour. Subjects who experience an infusion-related reaction preferably have the infusion rate reduced to half that rate, e.g., from 100 mg/hour to 50 mg/hour. Preferably, the infusion of such second or subsequent dose of anti-CD20 antibody (e.g., an about 1000-mg total dose) is completed by about 195 minutes (3 hours 15 minutes).

Aside from administration of antagonists to the patient by traditional routes as noted above, the present invention includes administration by gene therapy. Such administration of nucleic acids encoding the antagonist is encompassed by the expression “administering an effective amount of an antagonist”. See, for example, WO 1996/07321 concerning the use of gene therapy to generate intracellular antibodies.

There are two major approaches to getting the nucleic acid (optionally contained in a vector) into the patient's cells, in vivo and ex vivo. For in vivo delivery the nucleic acid is injected directly into the patient, usually at the site where the antagonist is required. For ex vivo treatment, the patient's cells are removed, the nucleic acid is introduced into these isolated cells, and the modified cells are administered to the patient either directly or, for example, encapsulated within porous membranes that are implanted into the patient (see, e.g. U.S. Pat. No. 4,892,538 and U.S. Pat. No. 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent specific for the target cells, such as an antibody specific for a cell-surface membrane protein on the target cell, a ligand for a receptor on the target cell, etc. Where liposomes are employed, proteins that bind to a cell-surface membrane protein associated with endocytosis may be used for targeting and/or to facilitate uptake, e.g. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262:4429-4432 (1987) and Wagner et al., Proc. Natl. Acad. Sci. USA, 87:3410-3414 (1990). Gene-marking and gene-therapy protocols are described, for example, in Anderson et al., Science, 256:808-813 (1992) and WO 1993/25673.

In another embodiment, a method is provided for treating joint damage in a subject eligible for treatment based on the biomarker analysis herein comprising administering a B-cell antagonist, such as an antibody thereto, for example, anti-CD20 antibody, to the subject, and giving the subject, at least about 52 weeks after the administration, an imaging test that measures a reduction in the joint damage as compared to baseline prior to the administration, wherein the amount of antagonist such as anti-CD20 antibody administered is effective in achieving a reduction in the joint damage, indicating that the subject has been successfully treated.

In this method, preferably the test measures a total modified Sharp score. In another preferred embodiment of this joint-treatment method, the antagonist is an anti-CD20, anti-CD22, or anti-BR-3 antibody or BR3-Fc. More preferably, the anti-CD20 antibody is the preferred such antibodies set forth above, including rituximab, GA101, TRU-015, and a 2H7 antibody as set forth above.

In another preferred embodiment, the joint damage is caused by arthritis, preferably RA, and more preferably early or incipient RA. In all the methods herein, the RA is preferably early or incipient RA. The subject herein may be RF negative or positive.

In another aspect, such method further comprises re-treating the subject by providing an additional administration to the subject of the antagonist such as an anti-CD20 antibody in an amount effective to treat RA or achieve a continued or maintained reduction in joint damage as compared to the effect of a prior administration of the antagonist. The re-treatment may be commenced at least about 24 weeks (preferably at about 24 weeks) after the first administration of the antagonist, and one or more further re-treatments is optionally commenced. In another embodiment, the further re-treatment is commenced at least about 24 weeks after the second administration of the antagonist.

In one aspect the antagonist is additionally administered to the subject even if there is no clinical improvement in the subject at the time of RA testing or another imaging testing after a prior administration.

In a further preferred aspect, RA or joint damage has been reduced after the re-treatment as compared to the extent of RA or joint damage after the first assessment such as imaging assessment.

If multiple exposures of antagonist are provided as in re-treatment, each exposure may be provided using the same or a different administration means. In one embodiment, each exposure is by i.v. administration. In another embodiment, each exposure is given by s.c. administration. In yet another embodiment, the exposures are given by both i.v. and s.c. administration.

Preferably the same antagonist, such as anti-CD20, anti-CD22, or anti-BR3 antibody, BR3-Fc, or TACI-Ig, is used for at least two antagonist exposures, and preferably for each antagonist exposure. Thus, the initial and second antagonist exposures are preferably with the same antagonist, and more preferably all antagonist exposures are with the same antagonist, i.e., treatment for the first two exposures, and preferably all exposures, is with one type of B-cell antagonist, e.g., an antagonist that binds to a B-cell surface marker, such as an anti-CD20 antibody, e.g., all with rituximab or all with the same 2H7 antibody.

Preferably, in this re-treatment method, a second medicament is administered in an effective amount, wherein the antagonist is a first medicament. In one aspect, the second medicament is more than one medicament. In another aspect, the second medicament is one of those set forth above, including an immunosuppressive agent, a DMARD, an integrin antagonist, a NSAID, a cytokine antagonist, a bisphosphonate, or a combination thereof, most preferably MTX.

For the re-treatment methods described herein, where a second medicament is administered in an effective amount with an antagonist exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such second medicament is reduced or eliminated so as to reduce the exposure of the subject to an agent with side effects such as prednisone, prednisolone, methylprednisolone, and cyclophosphamide.

In one embodiment of the re-treatment method, the subject has never been previously administered any drug(s), such as immunosuppressive agent(s), to treat the RA or joint damage. In another aspect, the subject or patient is responsive to previous therapy for the RA or joint damage.

In another aspect of re-treatment, the subject or patient has been previously administered one or more medicaments(s) to treat the RA or joint damage. In a further embodiment, the subject or patient was not responsive to one or more of the medicaments that had been previously administered. Such drugs to which the subject may be non-responsive include, for example, chemotherapeutic agents, immunosuppressive agents, cytokine antagonists, integrin antagonists, corticosteroids, analgesics, or B-cell antagonists such as antagonists to B-cell surface markers, for example, anti-CD20 antibody. More particularly, the drugs to which the subject may be non-responsive include immunosuppressive agents or B-cell antagonists such as anti-CD20 antibodies. Preferably, such antagonists are not antibodies or immunoadhesins, and are, for example, small-molecule inhibitors, or anti-sense oligonucleotides, or antagonistic peptides, as noted, for example, in the background section. In a further aspect, such antagonists include an antibody or immunoadhesin, such that re-treatment is contemplated with one or more antibodies or immunoadhesins of this invention to which the subject was formerly non-responsive. Most preferably, the subject or patient is not responsive to previous therapy with MTX or a TNF-α inhibitor.

In a further aspect, the invention involves a method of reducing the risk of a negative side effect in a subject (e.g., selected from the group consisting of an infection, cancer, heart failure, and demyelination) comprising administering to the subject an effective amount of a B-cell antagonist if the subject has one or more of the biomarkers herein.

A discussion of methods of producing, modifying, and formulating such antagonists follows.

IV. Production of Antagonists

The methods and articles of manufacture of the present invention use, or incorporate, a B-cell antagonist such as an antibody or immunoadhesin. Methods for screening for such antagonists are noted above. Methods for generating such antagonists are well within the skill of the art, and include chemical synthesis, recombinant production, hybridoma production, peptide synthesis, oligonucleotide synthesis, phage-display, etc., depending on the type of antagonist being produced.

B-cell surface antigens or B-cell specific proliferation or survival factors to be used for production of, or screening for, antagonist(s) may be, e.g., a soluble form of the antigen or proliferation/survival factor or a portion thereof, containing the desired epitope. Alternatively, or additionally, cells expressing the antigen at their surface, or expressing the B-cell specific survival/proliferation factor, can be used to generate, or screen for, antagonist(s). Other forms of B-cell surface markers and proliferation/survival factors useful for generating antagonists will be apparent to those skilled in the art.

While the preferred antagonist is an antibody or immunoadhesin, other antagonists are contemplated herein. For example, the antagonist may comprise a small-molecule antagonist optionally fused to, or conjugated with, a cytotoxic agent. Libraries of small molecules may be screened against the B-cell surface antigen or survival/proliferation factor of interest herein to identify a small molecule that binds to that antigen or factor. The small molecule may further be screened for its antagonistic properties and/or conjugated with a cytotoxic agent.

The antagonist may also be a peptide generated by rational design or by phage display (see, e.g., WO 98/35036). In one embodiment, the molecule of choice may be a “CDR mimic” or antibody analogue designed based on the CDRs of an antibody. While such peptide may be antagonistic by itself, the peptide may optionally be fused to a cytotoxic agent to add or enhance antagonistic properties of the peptide.

A description follows as to exemplary techniques for the production of the antibody antagonists used in accordance with the present invention.

(i) Polyclonal Antibodies

Polyclonal antibodies are preferably raised in animals by multiple s.c. or intraperitoneal (i.p.) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol (PEG), to form a hybridoma cell (see, for example, Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif., and SP-2 or X63-Ag8-653 cells available from the ATCC, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as RIA or ELISA.

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods. Goding, Monoclonal Antibodies: Principles and Practice, pp. 59-103 (Academic Press, 1986). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™ medium, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries. Waterhouse et al., Nuc. Acids. Res., 21:2265-2266 (1993). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human FR for the humanized antibody. Sims et al., J. Immunol., 151:2296 (1993); Chothia et al., J. Mol. Biol., 196:901 (1987). Another method uses a particular FR derived from the consensus sequence of all human antibodies of a particular subgroup of light- or heavy-chain variable regions. The same FR may be used for several different humanized antibodies. Carter et al., Proc. Natl. Acad. Sci. USA, 89:4285 (1992); Presta et al., J. Immunol., 151:2623 (1993).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the HVR residues are directly and most substantially involved in influencing antigen binding.

(iv) Human Antibodies

As an alternative to humanization, human antibodies can be generated. For example, transgenic animals (e.g., mice) can be generated that are capable, upon immunization, of producing a full repertoire of human antibodies in the absence of endogenous immunoglobulin production. The homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. No. 5,591,669; U.S. Pat. No. 5,589,369; and U.S. Pat. No. 5,545,807.

Alternatively, phage-display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson and Chiswell, Current Opinion in Structural Biology, 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991) or Griffith et al., EMBO J., 12:725-734 (1993). See also U.S. Pat. No. 5,565,332 and U.S. Pat. No. 5,573,905.

Human antibodies may also be generated by in-vitro activated B cells (see, for example, U.S. Pat. No. 5,567,610 and U.S. Pat. No. 5,229,275).

(v) Antibody Fragments

Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See WO 1993/16185; U.S. Pat. No. 5,571,894; and U.S. Pat. No. 5,587,458. The antibody fragment may also be a “linear antibody,” e.g., as described in U.S. Pat. No. 5,641,870. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the CD20 antigen. Other such antibodies may bind CD20 and further bind a second B-cell surface marker or B-cell specific proliferation/survival factor such as anti-CD22 antibodies. Alternatively, an anti-CD20 binding arm may be combined with an arm that binds to a triggering molecule on a leukocyte such as a T-cell receptor molecule (e.g., CD2 or CD3), or Fc receptors for IgG (FcγR), such as FcγRI (CD64), FcγRRII (CD32), and FcγRIII (CD16) so as to focus cellular-defense mechanisms to the B cell. Bispecific antibodies may also be used to localize cytotoxic agents to the B cell. These antibodies possess a CD20-binding arm and an arm that binds the cytotoxic agent (e.g., saporin, anti-interferon-α, vinca alkaloid, ricin A chain, MTX, or radioactive isotope hapten). Bispecific antibodies can be prepared as full-length antibodies or antibody fragments (e.g., F(ab′)₂-bispecific antibodies).

Methods for making bispecific antibodies are known in the art. Traditional production of full-length bispecific antibodies is based on the co-expression of two immunoglobulin heavy-chain/light-chain pairs, where the two chains have different specificities. Millstein et al., Nature, 305:537-539 (1983). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 1993/08829 and Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1), containing the site necessary for light-chain binding, present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy-chain/light-chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune-system cells to unwanted cells (U.S. Pat. No. 4,676,980), and for treatment of HIV infection (WO 1991/00360, WO 1992/020373, and EP 03089). Heteroconjugate antibodies may be made using any convenient cross-linking methods. Suitable cross-linking agents are well known in the art, and are disclosed, e.g., in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describes a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers (see, e.g., Kostelny et al., J. Immunol., 148(5):1547-1553 (1992)). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Holliger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. Gruber et al., J. Immunol., 152:5368 (1994).

Antibodies with more than two valencies are contemplated. For example, trispecific antibodies can be prepared (see, e.g., Tutt et al. J. Immunol., 147:60 (1991)).

V. Modifications of the Antagonist

Modifications of the antagonist are contemplated herein. For example, the antagonist may be linked to one of a variety of non-proteinaceous polymers, e.g., PEG, polypropylene glycol, polyoxyalkylenes, or copolymers of PEG and polypropylene glycol. Antibody fragments, such as Fab′, linked to one or more PEG molecules are a therapeutic embodiment of the invention.

The antagonists disclosed herein may also be formulated as liposomes. Liposomes containing the antagonist are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. No. 4,485,045 and U.S. Pat. No. 4,544,545; and WO 1997/38731. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidyl-ethanolamine (PEG-PE). Liposomes are extruded through filters of defined pore size to yield liposomes with the desired diameter. Fab′ fragments of an antibody of the present invention can be conjugated to the liposomes as described in Martin et al., J. Biol. Chem., 257:286-288 (1982) via a disulfide-interchange reaction. A chemotherapeutic agent is optionally contained within the liposome. See Gabizon et al., J. National Cancer Inst., 81(19):1484 (1989).

Amino acid sequence modification(s) of protein or peptide antagonists described herein is/are contemplated. For example, it may be desirable to improve the binding affinity and/or other biological properties of the antagonist. Amino acid sequence variants of the antagonist are prepared by introducing appropriate nucleotide changes into the antagonist-encoding nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the final construct, provided that the final construct possesses the desired characteristics. The amino acid changes also may alter post-translational processes of the antagonist, such as changing the number or position of glycosylation sites.

A useful method for identification of certain residues or regions of the antagonist that are preferred locations for mutagenesis is called “alanine-scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the N- or C-terminus of the antagonist of an enzyme, or a polypeptide that increases the serum half-life of the antagonist.

Another type of variant is an amino acid substitution variant. These variants have at least one amino acid residue in the antagonist molecule replaced by a different residue. The sites of greatest interest for substitutional mutagenesis of antibody antagonists include the hypervariable regions, but FR alterations are also contemplated. Conservative substitutions are shown in Table 1 under the heading of “preferred substitutions.” If such substitutions result in a change in biological activity, then more substantial changes, denominated “exemplary substitutions” in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; | leu |
| | phe; norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in the biological properties of the antagonist are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for a member of another class.

Any cysteine residue not involved in maintaining the proper conformation of the antagonist also may be substituted, generally with serine, to improve the oxidative stability of the molecule and prevent aberrant crosslinking. Conversely, cysteine bond(s) may be added to the antagonist to improve its stability (particularly where the antagonist is an antibody fragment such as an Fv fragment).

A particularly preferred type of substitutional variant involves substituting one or more HVR residues of a parent antibody. Generally, the resulting variant(s) selected for further development will have improved biological properties relative to the parent antibody from which they are generated. A convenient way for generating such substitutional variants is affinity maturation using phage display. Briefly, several HVR sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. Alanine-scanning mutagenesis can be performed to identify candidate HVR residues contributing significantly to antigen binding for possible modification. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. Such altering includes deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist is typically accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US 2003/0157108 (Presta). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878 (Jean-Mairet et al.) and U.S. Pat. No. 6,602,684 (Umana et al.). Antibodies with at least one galactose residue in the oligosaccharide attached to an Fc region of the antibody are reported in WO 1997/30087 (Patel et al.) See also WO 1998/58964 (Raju) and WO 1999/22764 (Raju) concerning antibodies with altered carbohydrate attached to the Fc region thereof. See also US 2005/0123546 (Umana et al.); US 2004/0072290 (Umana et al.); US 2003/0175884 (Umana et al.); WO 2005/044859 (Umana et al.) and US 2007/0111281 (Sondermann et al.) on antigen-binding molecules with modified glycosylation, including antibodies with an Fc region containing N-linked oligosaccharides; and US 2007/0010009 (Kanda et al.).

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein that further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; US 2006/0063254; US 2006/0064781; US 2006/0078990; US 2006/0078991; Okazaki et al., J. Mol. Biol., 336:1239-1249 (2004); and Yamane-Ohnuki et al., Biotech. Bioeng., 87:614 (2004). Examples of cell lines producing defucosylated antibodies include Lec13 CHO cells deficient in protein fucosylation (Ripka et al., Arch. Biochem. Biophys., 249:533-545 (1986); US 2003/0157108 A1 (Presta); and WO 2004/056312 (Adams et al., especially at Example 11), and knockout cell lines, such as the alpha-1,6-fucosyltransferase gene, FUT8, knockout CHO cells (Yamane-Ohnuki et al., Biotech. Bioeng., 87:614 (2004)). See also Kanda et al., Biotechnol. Bioeng., 94:680-688 (2006). US 2007/0048300 (Biogen-IDEC) discloses a method of producing aglycosylated Fc-containing polypeptides, such as antibodies, having a desired effector function, as well as aglycosylated antibodies produced according to the method, and methods of using such antibodies as therapeutics. See also U.S. Pat. No. 7,262,039, which relates to a polypeptide having an alpha-1,3-fucosyltransferase activity, including a method for producing a fucose-containing sugar chain using the polypeptide.

See also US 2006/024304 (Gerngross et al.); U.S. Pat. No. 7,029,872 (Gerngross); US 2004/018590 (Gerngross et al.); US 2006/034828 (Gerngross et al.); US 2006/034830 (Gerngross et al.); US 2006/029604 (Gerngross et al.); WO 2006/014679 (Gerngross et al.); WO 2006/014683 (Gerngross et al.); WO 2006/014685 (Gerngross et al.); WO 2006/014725 (Gerngross et al.); WO 2006/014726 (Gerngross et al.); and US 2007/0248600/WO 2007/115813 (Hansen et al.) on recombinant glycoproteins and glycosylation variants.

Nucleic acid molecules encoding amino-acid-sequence variants of the antagonist are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist.

It may be desirable to modify the antagonist used herein with respect to effector function, e.g., so as to enhance ADCC and/or CDC of the antagonist. This may be achieved by introducing one or more amino acid substitutions into an Fc region of an antibody antagonist. Alternatively or additionally, cysteine residue(s) may be introduced into the Fc region, thereby allowing interchain disulfide bond formation in this region. The homodimeric antibody thus generated may have improved internalization capability and/or increased complement-mediated cell killing and ADCC. See Caron et al., J. Exp Med., 176:1191-1195 (1992) and Shopes, J. Immunol., 148:2918-2922 (1992). Homodimeric antibodies may also be prepared using heterobifunctional cross-linkers as described in Wolff et al., Cancer Research, 53:2560-2565 (1993). Alternatively, an antibody can be engineered that has dual Fc regions and may thereby have enhanced complement lysis and ADCC capabilities. See Stevenson et al., Anti-Cancer Drug Design, 3:219-230 (1989). WO 2000/42072 (Presta, L.) describes antibodies with improved ADCC function in the presence of human effector cells, where the antibodies comprise amino acid substitutions in the Fc region thereof.

Antibodies with altered C1q binding and/or CDC are described in WO 1999/51642 and U.S. Pat. No. 6,194,551, U.S. Pat. No. 6,242,195, U.S. Pat. No. 6,528,624, and U.S. Pat. No. 6,538,124 (Idusogie et al.). The antibodies comprise an amino acid substitution at one or more of amino acid positions 270, 322, 326, 327, 329, 313, 333, and/or 334 of the Fc region thereof.

To increase the serum half life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO 2000/42072 (Presta, L.).

Engineered antibodies with three or more (preferably four) functional antigen binding sites are also contemplated. See US 2002/0004587, Miller et al.

VI. Pharmaceutical Formulations

Therapeutic formulations of the antagonists used in accordance with the present invention are prepared for storage by mixing the antagonist having the desired degree of purity with optional pharmaceutically acceptable carriers, excipients, or stabilizers in the form of lyophilized formulations or aqueous solutions. For general information concerning formulations, see, e.g., Gilman et al. (eds.), The Pharmacological Bases of Therapeutics, 8th Ed. (Pergamon Press, 1990); Gennaro (ed.), Remington's Pharmaceutical Sciences, 18th Edition (Mack Publishing Co., Easton, Pa., 1990); Avis et al. (eds.), Pharmaceutical Dosage Forms: Parenteral Medications (Dekker, New York, 1993); Lieberman et al. (eds.), Pharmaceutical Dosage Forms: Tablets (Dekker, New York, 1990); Lieberman et al. (eds.) Pharmaceutical Dosage Forms: Disperse Systems (Dekker, New York, 1990); and Walters (ed.), Dermatological and Transdermal Formulations (Drugs and the Pharmaceutical Sciences), Vol 119 (Dekker, New York, 2002).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as ethylenediaminetetraacetic acid (EDTA); sugars such as sucrose, mannitol, trehalose, or sorbitol; salt-forming counter-ions such as sodium; metal complexes (e.g., Zn-protein complexes); and/or non-ionic surfactants such as TWEEN™, PLURONICS™, or PEG.

Exemplary anti-CD20 antibody formulations are described in WO 1998/56418, which describes a liquid multidose formulation comprising 40 mg/mL rituximab, 25 mM acetate, 150 mM trehalose, 0.9% benzyl alcohol, and 0.02% POLYSORBATE 20™ at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL POLYSORBATE 80™, and Sterile Water for Injection, pH 6.5.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antagonist are also contemplated. See, for example, US 2002/0136719A1 (Shenoy et al.).

The formulation herein may also contain more than one active compound (a second medicament as noted above), preferably those with complementary activities that do not adversely affect each other. The type and effective amounts of such medicaments depend, for example, on the amount and type of B-cell antagonist present in the formulation, and clinical parameters of the subjects. The preferred such second medicaments are noted above.

The active ingredients may also be entrapped in microcapsules prepared, e.g., by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra, for example.

Sustained-release formulations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

VII. Articles of Manufacture

For use in detection of the biomarkers, kits or articles of manufacture are also provided by the invention. Such kits can be used to determine if a subject with RA will be effectively responsive to a B-cell antagonist. These kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a protein or autoantibody marker or a PTPN22 or SE gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, e.g., avidin or streptavidin, bound to a reporter molecule, such as an enzymatic, florescent, or radioisotope label.

Such kit will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above.

The kits of the invention have a number of embodiments. A typical embodiment is a kit comprising a container, a label on the container, and a composition contained within the container, wherein the composition includes one or more polynucleotides that hybridize to a complement of the PTPN22 SNP and/or of the SE under stringent conditions, and the label on the container indicates that the composition can be used to evaluate the presence of PTPN22 SNP and/or SE in a sample, and wherein the kit includes instructions for using the polynucleotide(s) for evaluating the presence of the SNP and/or SE RNA or DNA in a particular sample type.

Another aspect is a kit comprising a container, a label on the container, and a composition contained within the container, wherein the composition includes a primary antibody that binds to a protein or autoantibody biomarker, and the label on the container indicates that the composition can be used to evaluate the presence of such proteins or antibodies in a sample, and wherein the kit includes instructions for using the antibody for evaluating the presence of biomarker proteins in a particular sample type. The kit can further comprise a set of instructions and materials for preparing a sample and applying antibody to the sample. The kit may include both a primary and secondary antibody, wherein the secondary antibody is conjugated to a label, e.g., an enzymatic label.

Other optional components of the kit include one or more buffers (e.g., block buffer, wash buffer, substrate buffer, etc.), other reagents such as substrate (e.g., chromogen) that is chemically altered by an enzymatic label, epitope retrieval solution, control samples (positive and/or negative controls), control slide(s), etc. Kits can also include instructions for interpreting the results obtained using the kit.

In further specific embodiments, for antibody-based kits, the kit can comprise, for example: (1) a first antibody (e.g., attached to a solid support) that binds to a biomarker protein; and, optionally, (2) a second, different antibody that binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a biomarker protein or (2) a pair of primers useful for amplifying a biomarker nucleic acid molecule. The kit can also comprise, e.g., a buffering agent, a preservative, or a protein-stabilizing agent. The kit can further comprise components necessary for detecting the detectable label (e.g., an enzyme or a substrate). The kit can also contain a control sample or a series of control samples that can be assayed and compared to the test sample. Each component of the kit can be enclosed within an individual container, and all of the various containers can be included within a single package, along with instructions for interpreting the results of the assays performed using the kit.

Also provided by the invention are articles of manufacture containing materials useful for the treatment of the RA. The article of manufacture comprises a container and a label or package insert on or associated with the container. In this aspect, the package insert is on or associated with the container. Suitable containers include, for example, bottles, vials, syringes, etc. The containers may be formed from a variety of materials such as glass or plastic. The container holds or contains the antagonist that is effective for treating the RA and may have a sterile access port (for example, the container may be an intravenous solution bag or a vial having a stopper pierceable by a hypodermic injection needle). At least one active agent in the composition is the B-cell antagonist. The label or package insert indicates that the composition is used for treating RA in a subject eligible for treatment with specific guidance regarding dosing amounts and intervals of antagonist and any other medicament being provided.

The article of manufacture may further comprise a second container comprising a pharmaceutically acceptable diluent buffer, such as bacteriostatic water for injection (BWFI), phosphate-buffered saline, Ringer's solution, and dextrose solution. The article of manufacture may further include other materials desirable from a commercial and user standpoint, including other buffers, diluents, filters, needles, and syringes.

The kits and articles of manufacture herein also include information, for example in the form of a package insert or label, indicating that the composition is used for treating RA where the genotype(s) showing the polymorphism and/or SE herein are detected in a genetic sample from the patient with the disease. Optionally, the label or package insert may indicate that other suitable biomarkers can be detected, such as seropositivity for anti-CCP and/or RF, in addition to the presence of one or both of the SNP or SE. The insert or label may take any form, such as paper or electronic media, for example, a magnetically recorded medium (e.g., floppy disk) or a CD-ROM. The label or insert may also include other information concerning the pharmaceutical compositions and dosage forms in the kit or article of manufacture.

Generally, such information aids patients and physicians in using the enclosed pharmaceutical compositions and dosage forms effectively and safely. For example, the following information regarding the antagonist may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, and patent information.

In a specific embodiment of the invention, an article of manufacture is provided comprising, packaged together, a pharmaceutical composition comprising a B-cell antagonist and a pharmaceutically acceptable carrier and a label stating that the antagonist or pharmaceutical composition is indicated for treating patients with RA from which a genetic sample has been obtained showing the presence of a PTPN22 R620W SNP and/or SE. This can be shown by assessing genetic expression as a biomarker of a PTPN22 R620W SNP and/or SE. Further, the label may indicate that additional appropriate biomarkers may be assessed, for example, seropositivity for one or both of anti-CCP and RF. The same method can apply to joint damage.

In a preferred embodiment the article of manufacture herein further comprises a container comprising a second medicament, wherein the antagonist is a first medicament, and which article further comprises instructions on the package insert for treating the patient with the second medicament in an effective amount. The second medicament may be any of those set forth above, including an immunosuppressive agent, a corticosteroid, a DMARD, an integrin antagonist, a NSAID, a cytokine antagonist, a bisphosphonate, or a combination thereof, more preferably a DMARD, NSAID, cytokine antagonist, integrin antagonist, or immunosuppressive agent. Most preferably, the second medicament is MTX.

Also the invention provides a method for manufacturing a B-cell antagonist or a pharmaceutical composition thereof comprising combining in a package the antagonist or pharmaceutical composition and a label stating that the antagonist or pharmaceutical composition is indicated for treating patients with RA from which a genetic sample has been obtained showing the presence of a PTPN22 R620W SNP or SE or both. This can be shown by assessing genetic expression as a biomarker of a PTPN22 R620W SNP and/or SE. The label may also indicate that additional suitable biomarkers may be assessed, e.g., seropositivity for one or both of anti-CCP and RF. The same method can apply to joint damage.

The invention also supplies a method of providing a treatment option for patients with RA comprising packaging a B-cell antagonist in a vial with a package insert containing instructions to treat patients with RA from whom a genetic sample has been obtained showing the presence of a PTPN22 R620W SNP or SE, or both SNP and SE. The same method can apply to joint damage.

VIII. Methods of Advertising

The invention herein also encompasses a method for advertising a B-cell antagonist or a pharmaceutically acceptable composition thereof comprising promoting, to a target audience, the use of the antagonist or pharmaceutical composition thereof for treating a patient or patient population with RA from whom a genetic sample has been obtained showing the presence of a PTPN22 R620W SNP or SE, or both SNP and SE. This can be shown by assessing genetic expression as a biomarker of a PTPN22 R620W SNP or SE, or both SNP and SE. The method optionally comprises additionally assessing other biomarkers, including seropositivity for one or both of anti-CCP and RF. The same method can apply to joint damage.

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the diagnostic and treatment methods herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store public announcement (PA) systems, or anywhere a visual or audible communication can be placed. More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments and diagnostics. The advertising may be individualized or customized based on user characterizations defined by service interaction and/or other data such as user demographics and geographical location.

Many alternative experimental methods known in the art may be successfully substituted for those specifically described herein in the practice of this invention, such as, for example. described in manuals, textbooks and websites available in the areas of technology relevant to this invention (e.g., Using Antibodies, A Laboratory Manual, Harlow, E. and Lane, D., eds. (Cold Spring Harbor Laboratory Press, New York, 1999); Roe et. al., DNA Isolation and Sequencing (Essential Techniques Series) (John Wiley & Sons, 1996); Methods in Enzymology: Chimeric Genes and Proteins, Abelson et al., eds. (Academic Press, 2000); Molecular Cloning: a Laboratory Manual, 3rd Edition, by Sambrook and MacCallum, (Cold Spring Harbor Laboratory Press, New York, 2001); Current Protocols in Molecular Biology, Ausubel et. al., eds. (John Wiley & Sons, 1987) and periodic updates; PCR: The Polymerase Chain Reaction, (Mullis et al., ed., 1994); Current Protocols in Protein Science, Coligan, ed. (John Wiley & Sons, 2003); and Methods in Enzymology: Guide to Protein Purification, Vol. 182, Deutscher, ed. (Academic Press, Inc., 1990)).

Further details of the invention are illustrated by the following non-limiting Examples. The disclosures of all citations in the specification are expressly incorporated herein by reference.

### EXAMPLES

### Statistical Methods

The statistical tasks can comprise the following steps:
1. Pre-selection of candidate biomarkers
2. Pre-selection of relevant clinical efficacy response predictive covariates
3. Selection of biomarker prediction functions at a univariate level
4. Selection of biomarker prediction functions, including clinical covariates at a univariate level
5. Selection of biomarker prediction functions at a multivariate level
6. Selection of biomarker prediction functions, including clinical covariates at a multivariate level

The following text details the different steps:

1: Pre-selection of candidate biomarkers: The statistical pre-selection of candidate biomarkers is oriented towards the strength of association with measures of clinical benefit. For this purpose the different clinical endpoints may be transformed into derived surrogate scores, as, e.g., an ordinal assignment of the degree of clinical benefit scores regarding time to progression (TTP) that avoid censored observations. These surrogate transformed measures can be easily used for simple correlation analysis, e.g., by the non-parametric Spearman rank correlation approach. An alternative is to use the biomarker measurements as metric covariates in time-to-event regression models, as, e.g., Cox proportional hazard regression. Depending on the statistical distribution of the biomarker values, this step may require some pre-processing, as, for example, variance-stabilizing transformations and the use of suitable scales or, alternatively, a standardization step such as using percentiles instead of raw measurements. A further approach is inspection of bivariate scatter plots, for example, by displaying the scatter of x-axis=biomarker value, y-axis=measure of clinical benefit) on a single-patient basis. Some non-parametric regression line, as achieved, for example, by smoothing splines, can be useful to visualize the association of biomarker and clinical benefit.

The goal of these different approaches is the pre-selection of biomarker candidates that show some association with clinical benefit in at least one of the benefit measures employed, while results for other measures are not contradictory. When there are available control groups, the differences in association of biomarkers with clinical benefit in the different arms could be a sign of differential prediction that makes the biomarker(s) eligible for further consideration.

2: Pre-selection of relevant clinical efficacy response predictive covariates: The statistical pre-selection of clinical covariates as defined herein parallels the approaches for pre-selecting biomarkers and is also oriented towards the strength of association with measures of clinical benefit. So, in principle, the same methods apply as considered under point 1 above. In addition to statistical criteria, criteria from clinical experience and theoretical knowledge may apply to pre-select relevant clinical covariates.

The predictive value of clinical covariates could interact with the predictive value of the biomarkers. They will be considered for refined prediction rules, if necessary.

3: Selection of biomarker prediction functions at a univariate level: The term “prediction function” will be used in a general sense to mean a numerical function of a biomarker measurement that results in a number scaled to imply the target prediction.

A simple example is the choice of the Heaviside function for a specific cutoff c and a biomarker measurement x, where the binary prediction A or B is to be made, then

If H(x−c)=0, then predict A.

If H(x−c)=1, then predict B.

This is probably the most common way of using univariate biomarker measurements in prediction rules. The definition of “prediction function” as noted above includes referral to an existing training data set that can be used to explore the prediction possibilities. Different routes can be taken to achieve a suitable cutoff c from the training set. First, the scatterplot with smoothing spline mentioned under point 1 can be used to define the cutoff. Alternatively, some percentile of the distribution could be chosen, e.g., the median or a quartile. Cutoffs can also be systematically extracted by investigating all possible cutoffs according to their prediction potential with regard to the measures of clinical benefit. Then, these results can be plotted to allow for a manual selection or to employ some search algorithm for optimality. This can be realized based on certain clinical endpoints using a Cox model, wherein at each test cutoff the biomarker is used as a binary covariate. Then the results for the clinical endpoints can be considered together to choose a cutoff that shows prediction in line with both endpoints.

Another uncommon approach for choosing a prediction function can be based on a fixed-parameter Cox regression model obtained from the training set with biomarker values (possibly transformed) as covariate. A further possibility is to base the decision on some likelihood ratio (or monotonic transform of it), where the target probability densities are pre-determined in the training set for separation of the prediction states. Then the biomarker would be plugged into some function of predictive criteria.

4: Selection of biomarker prediction functions including clinical covariates at a univariate level: Univariate refers to using only one biomarker—with regard to clinical covariates, this can be a multivariate model. This approach parallels the search without clinical covariates, except that the methods should allow for incorporating the relevant covariate information. The scatterplot method of choosing a cutoff allows only a limited use of covariates, e.g., a binary covariate could be color coded within the plot. If the analysis relies on some regression approach, then the use of covariates (also many of them at a time) is usually facilitated. The cutoff search based on the Cox model described under point 3 above allows for an easy incorporation of covariates and thereby leads to a covariate-adjusted univariate cutoff search. The adjustment by covariates may be done as covariates in the model or via the inclusion in a stratified analysis.

Also, the other choices of prediction functions allow for the incorporation of covariates.

This is straightforward for the Cox model choice as prediction function. This includes the option to estimate the influence of covariates on an interaction level, which means that, e.g., for different age groups different predictive criteria apply.

For the likelihood ratio type of prediction functions, the prediction densities must be estimated including covariates. For this purpose, the methodology of multivariate pattern recognition can be used or the biomarker values can be adjusted by multiple regression on the covariates (prior to density estimation).

The CART technology (Classification and Regression Trees, Breiman et al. (Wadsworth, Inc.: New York, 1984) can be used for this purpose, employing a biomarker (raw measurement level) plus clinical covariates and utilizing a clinical benefit measure as response. Cutoffs are searched and a decision-tree type of function will be found involving the covariates for prediction. The cutoffs and algorithms chosen by CART are frequently close to optimal and may be combined and unified by considering different clinical benefit measures.

5: Selection of biomarker prediction functions at a multivariate level: When there are several biomarker candidates that maintain their prediction potential within the different univariate prediction function choices, then a further improvement may be achieved by combinations of biomarkers, i.e., considering multivariate prediction functions.

Based on the simple Heaviside function model, combinations of biomarkers may be evaluated, e.g., by considering bivariate scatterplots of biomarker values where optimal cutoffs are indicated. Then a combination of biomarkers can be achieved by combining different Heaviside functions by the logical “AND” and “OR” operators to achieve an improved prediction.

The CART technology can be used for this purpose, employing multiple biomarkers (raw measurement level) and a clinical benefit measure as response, to achieve cutoffs for biomarkers and decision-tree type of functions for prediction. The cutoffs and algorithms chosen by CART are frequently close to optimal and may be combined and unified by considering different clinical benefit measures.

The Cox-regression can be employed on different levels. A first way is to incorporate the multiple biomarkers in a binary way (i.e., based on Heaviside functions with some cutoffs). Another option is to use biomarkers in a metric way (after suitable transformations), or use a mixture of the binary and metric approaches. The evolving multivariate prediction function is of the Cox type described under point 3 above.

The multivariate likelihood ratio approach is difficult to implement, but presents another option for multivariate prediction functions.

6: Selection of biomarker prediction functions including clinical covariates at a multivariate level: When there are relevant clinical covariates, then a further improvement may be achieved by combining multiple biomarkers with multiple clinical covariates. The different prediction function choices will be evaluated with respect to the possibilities to include clinical covariates.

Based on the simple logical combinations of Heaviside functions for the biomarkers, further covariates may be added to the prediction function based on the logistic regression model obtained in the training set.

The CART technology and the evolving decision trees can be easily used with additional covariates, which would include those in the prediction algorithm.

All prediction functions based on the Cox-regression can use further clinical covariates. The option exists to estimate the influence of covariates on an interaction level, which means that, e.g., for different age groups different predictive criteria apply.

The multivariate likelihood ratio approach is not directly extendible to the use of additional covariates.

### Example 1

In this example, the exploratory cut-points noted above are used to assess the univariate effect of the factor groupings on different measures of the clinical benefit of rituximab or 2H7 antibody treatment (both available from Genentech) on RA patients, using degree of clinical efficacy response as an alternative clinical endpoint. Significant effects are expected to be observed for PTPN22 SNP and SE in log-rank tests for clinical efficacy response, as measured by ACR values (ACR20, 50, and 70).

The results are expected to show the pronounced effect of a grouping based on each of these biomarkers on the clinical outcome of the patients treated with rituximab or 2H7 antibody, as measured by clinical efficacy response.

### Example 2

Data in the literature noted above links SE and PTPN22 to CCP antibodies and RF. Tak et al., supra, notes data from the REFLEX and DANCER clinical trials showing that patients that were double negative for anti-CCP and RF exhibited less robust 6-month efficacy responses to rituximab. For the DANCER Phase 2b trial, see, for example, Emery et al., Arthr. and Rheum., 54:1390-1400 (2006) and for the REFLEX Phase III trial, see, for example, Cohen et al. Arthr. and Rheum., 54:2793-2806 (2006).

In this example, multivariate approaches are used to identify combinations of factors that would further improve the identification of RA patients with greater clinical benefit from the treatment with rituximab or 2H7 antibody. Results, as derived from a CART approach, are reflected. The CART classification approach makes it necessary to specify as the benefit group all values in clinical benefit above 0. As variables, serum levels of anti-CCP and RF are employed, as well as genetic expression of SE and the PTPN22 R620W SNP. Various combinations of SE and/or PTPN22 R620W SNP with serum anti-CCP and/or serum RF levels are selected to give best results. From the CART results, optimized cut-points for a combination of one or more of the genetic markers with serum anti-CCP and/or serum RF levels are derived.

The results are expected to demonstrate the significant effect of the grouping based on a combination of the SNP and/or SE with anti-CCP and/or RF on the clinical outcome of the patients treated with rituximab or 2H7 antibody, as measured by clinical efficacy response as described in Example 1 above.

### Example 3

A blood sample is obtained, with informed consent, from one or more patients with RA. DNA and serum/plasma are isolated, according to well known procedures.

The presence of PTPN22 CT/TT genotype in the sample is assessed as follows: DNA is isolated by standard methodologies from peripheral whole-blood samples. Genotyping of the PTPN22 SNP (rs2476601, 1858C-T, R620W) is performed using a PSQ HS 96A PYROSEQUENCER™ device. Briefly, 2 ng of DNA is amplified by PCR in a 10-ml reaction using the following primers:

(SEQ ID NO: 29)

| | |
|---|---|
| Forward: | 5′-GTTGCGCAGGCTAGTCTTGA-3′ |

(SEQ ID NO: 30)

| | |
|---|---|
| Reverse: | 5′-GCT GCT CCG GTT CAT AGA TT |
| | GGATAGCAACTGCTCCAAGG-3′ |

(SEQ ID NO: 31)

| | |
|---|---|
| Univ1_B | 5′-Biotin-GCT GCT CCG GTT CAT AGA TT-3′ |

The addition of specific sequences to the 5′ end of the reverse primer (shown in italics) allows the use of a biotinylated universal primer called Univ1_B and noted above. These primers are used at a ratio of 1:9 (reverse:universal primer). PCR conditions are as follows: 95° C.-12 min., 50 (95° C.-45 sec., 56.4° C.-45 sec., 72° C.-45 sec.), 72° C.-10 min., 4° C. forever. The amplicon is denatured with sodium hydroxide, separated, washed, and neutralized. The sequencing primer: 5′-AAATGATTCAGGTGTCC-3′ (SEQ ID NO:32) is used in combination with appropriate pyrosequencing substrates and enzymes according to the manufacturer's instructions to detect the polymorphism.

The presence of SE in the sample is assessed as follows: The HLA-DRB1 subtyping is performed by PCR using specific primers and hybridization with sequence-specific oligonucleotides. The SE alleles are HLA-DRB1 *0101, *0102, *0401, *0404, *0405, *0408, *0409, *0410, and *1001. HLA-DRB1 typing and subtyping are also performed using PCR-based methods, with the following alleles being classified as SE positive: DRB1 *0101, *0102, *0104, *0401, *0404, *0405, *0408, *0413, *0416, *1001, *1402, and *1406.

Where SE and/or PTPN22 CT/TT genotype are detected, the patient is treated with rituximab or 2H7 antibody using a dosing regimen selected from 375 mg/m² weekly×4, 500 mg×2 (on days 1 and 15), 1000 mg×2 (on days 1 and 15), or 1 gram×3 (on days 1, 15, and 29).

Patients may also receive concomitant MTX (10-25 mg/week per oral (p.o.) or parenteral), or other concomitant DMARD therapy. Patients may also receive folate (5 mg/week) given as either a single dose or as divided daily doses. Patients optionally continue to receive any background corticosteroid 10 mg/d prednisone or equivalent) throughout the treatment period.

The primary endpoint may be the proportion of patients with an ACR20 response at Week 24 using a Cochran-Mantel-Haenszel (CMH) test for comparing group differences, adjusted for relevant covariates including RF, anti-CCP, age, sex, etc.

Potential secondary endpoints include:

1. Proportion of patients with ACR50 and/or ACR70 responses at Week 24. These may be analyzed as specified for the primary endpoint.

2. Change in Disease Activity Score (DAS) from screening to Week 24. These may be assessed using an ANOVA model with baseline DAS, RF, and treatment as terms in the model.

3. Categorical DAS responders (EULAR response) at Week 24. These may be assessed using a CMH test adjusted for RF.

4. Changes from screening in ACR core set (SJC, TJC, patient's and physician's global assessments, HAQ, pain, CRP, and ESR). Descriptive statistics may be reported for these parameters.

5. Changes from screening in SF-36. Descriptive statistics may be reported for the 8 domain scores and the mental and physical component scores. In addition, the mental and physical component scores may be further categorized and analyzed.

6. Change in modified Sharp radiographic total score, erosion score, and joint space-narrowing score. These may be analyzed using continuous or categorical methodology, as appropriate.

Exploratory endpoints and analysis may involve:

ACR (20/50/70 and ACR n) and change in DAS responses over Weeks 8, 12, 16, 20, 24 and beyond will be assessed using a binary or continuous repeated measures model, as appropriate. Exploratory radiographic analyses including proportion of patients with no erosive progression may be assessed at weeks 24 and beyond.

Further exploratory endpoints (for example, complete clinical response, disease-free period) will be analyzed descriptively as part of the extended observation period.

Changes from Screen in FACIT-F fatigue will be analyzed with descriptive statistics.

Therapy of RA with rituximab or 2H7 antibody in patients with SE and/or PTPN22 CT/TT genotype as described above is expected to result in a superior clinical efficacy response according to any one or more of the endpoints noted above, and particularly to result in a higher clinical response than if the patients do not have these markers (e.g., ACR70 instead of ACR20 or ACR50 instead of ACR20).

The patients may also be assessed for anti-CCP levels and RF levels by ELISA using a standard commercial assay such as that sold by Inova Diagnostics. If the patients are positive for one or both of these biomarkers, as well as the SE and/or PTPN22 CT/TT genotype markers, they are treated with rituximab or 2H7 antibody as described above. Therapy of RA with either of these anti-CD20 antibodies in patients with SE and/or PTPN22 CT/TT genotype and positive levels of anti-CCP and/or RF as described above is expected to result in a beneficial clinical response according to any one or more of the endpoints noted above, and particularly to result in a higher clinical response than if the patients do not have these markers (e.g., ACR70 instead of ACR20 or ACR50 instead of ACR20). Thus, these biomarkers are expected to serve as a diagnostic for patients most likely to benefit from anti-CD20 antibody therapies.

In patients selected on the basis of the above biomarkers, rituximab or another anti-CD 20 antibody is expected to exhibit superior efficacy compared to patients negative for the above biomarkers, for treatment of RA and for induction and maintenance of joint damage remission in such patients with the claimed markers. Such anti-CD20 antibodies offer substantial advantages over standard therapy by virtue of their superior side-effect profiles, e.g., much less toxic than steroids, and better at restoring tolerance.

It is expected that the patients positively diagnosed and in the treatment arm will tolerate rituximab and 2H7 antibody infusions well and that their B cells will be depleted swiftly.

It is also expected that the patient diagnosed and treated with anti-CD20 antibodies such as rituximab or 2H7 antibody using a clinical protocol based on the parameters described in this specification and as known to those skilled in this art will show clinical improvement in the signs or symptoms of RA as evaluated by any one or more of the primary or secondary efficacy endpoints known for treating this disease. Moreover, the patient who is resistant or refractory to an immunosuppressive agent or another biological agent and who is treated, using a clinical protocol based on various parameters as described in this specification and as known to those skilled in the art, with the anti-CD20 antibody alone or in combination with a second medicament appropriate for the disease is expected to show greater improvement in any of the signs or symptoms of the RA, compared to the patient who continues on with the medicament to which he or she is resistant or refractory, or compared to the patient who is treated with only the second medicament appropriate for the disease and not with the anti-CD20 antibody.

### Example 4

In this example, the exploratory cut-points noted above are used to assess the univariate effect of the factor groupings on different measures of the clinical benefit of treatment with a humanized anti-CD22 antibody (epratuzumab (U.S. Pat. No. 6,183,744)) on RA patients, using degree of clinical efficacy response as an alternative clinical endpoint. Significant effects are expected to be observed for PTPN22 SNP and SE in log-rank tests for clinical efficacy response, as measured by ACR values (ACR20, 50, and 70).

The results are expected to show the pronounced effect of a grouping based on the SNP or SE or both on the clinical outcome of the patients treated with humanized anti-CD22 antibody, as measured by clinical efficacy response.

### Example 5

In this example, the exploratory cut-points noted above are used to assess the univariate effect of the factor groupings on different measures of the clinical benefit of anti-BR 3 antibody treatment on RA patients, using degree of clinical efficacy response as an alternative clinical endpoint. Suitable anti-BR3 antibodies for this purpose can be prepared, for example, as described in WO 2003/14294 and US 2005/0070689. Significant effects are expected to be observed for PTPN22 SNP and SE in log-rank tests for clinical efficacy response, as measured by ACR values (ACR20, 50, and 70).

The results are expected to show the pronounced effect of a grouping based on the SNP or SE or both on the clinical outcome of the patients treated with anti-BR3 antibody, as measured by clinical efficacy response.

### Example 6

In this example, the exploratory cut-points noted above are used to assess the univariate effect of the factor groupings on different measures of the clinical benefit of treatment with BR3-Fc or other BAFF antagonists on RA patients, using degree of clinical efficacy response as an alternative clinical endpoint. A suitable BR3-Fc immunoadhesin for this purpose is described in US 2005/0095243, US 2005/0163775, WO 2003/14294, and US 2005/0070689. Significant effects are expected to be observed for PTPN22 SNP and SE in log-rank tests for clinical efficacy response, as measured by ACR values (ACR20, 50, and 70).

The results are expected to show the pronounced effect of a grouping based on the SNP or SE or both on the clinical outcome of the patients treated with BR3-Fc or other BAFF antagonists as set forth in US 2005/0163775, WO 2003/14294, and US 2005/0070689, as measured by clinical efficacy response.

### Example 7

In this example, the exploratory cut-points noted above are used to assess the univariate effect of the factor groupings on different measures of the clinical benefit of treatment with atacicept (a TACI-Ig immunoadhesin, ZymoGenetics; see also Gross et al., Immunity, 15:289-291 (2001) and US 2007/0071760) on RA patients, using degree of clinical efficacy response as an alternative clinical endpoint. Significant effects are expected to be observed for PTPN22 SNP and SE in log-rank tests for clinical efficacy response, as measured by ACR values (ACR20, 50, and 70).

The results are expected to show the pronounced effect of a grouping based on the SNP or SE or both on the clinical outcome of the patients treated with atacicept, as measured by clinical efficacy response.

### Example 8

A blood sample is obtained, with informed consent, from one or more patients with RA. DNA and serum/plasma are isolated, according to well known procedures.

The presence of PTPN22 CT/TT genotype and SE in the sample is assessed as described in Example 3 above.

Where SE and/or PTPN22 CT/TT genotype are detected, the patient is treated with anti-CD22 antibody (epratuzumab from Immunomedics), or anti-BR3 antibody, or BR3-Fc (US 2005/0095243, US 2005/0163775, WO 2003/14294, and US 2005/0070689), or atacicept, or other BAFF or APRIL antagonists as set forth in these applications and/or as described above, using a dosing regimen selected from 375 mg/m² weekly×4, 500 mg×2 (on days 1 and 15), 1000 mg×2 (on days 1 and 15), or 1 gram×3 (on days 1, 15, and 29).

Patients may also receive concomitant MTX (10-25 mg/week per oral (p.o.) or parenteral), or other concomitant DMARD therapy. Patients may also receive folate (5 mg/week) given as either a single dose or as divided daily doses. Patients optionally continue to receive any background corticosteroid (10 mg/d prednisone or equivalent) throughout the treatment period.

The primary endpoint, potential secondary endpoints, and exploratory endpoints and analysis are those described in Example 3 above. Changes from Screen in FACIT-F fatigue will be analyzed with descriptive statistics.

Therapy of RA with anti-CD22 antibody, anti-BR3 antibody, BR3-Fc, atacicept, or other BAFF or APRIL antagonists in patients with SE and/or PTPN22 CT/TT genotype as described above is expected to result in a superior clinical efficacy response according to any one or more of the endpoints noted above, and particularly to result in a higher clinical response than if the patients do not have these markers (e.g., ACR70 instead of ACR20 or ACR50 instead of ACR20).

The patients may also be assessed for anti-CCP levels and RF levels by ELISA using a standard commercial assay such as that sold by Inova Diagnostics. If the patients are positive for one or both of these biomarkers, as well as the SE and/or PTPN22 CT/TT genotype markers, they are treated with anti-CD22 antibody, anti-BR3 antibody, BR3-Fc, atacicept, or other BAFF or APRIL antagonists as described above. Therapy of RA with any of these B-cell antagonists in patients with SE and/or PTPN22 CT/TT genotype and positive levels of anti-CCP and/or RF as described above is expected to result in a beneficial clinical response according to any one or more of the endpoints noted above, and particularly to result in a higher clinical response than if the patients do not have these markers (e.g., ACR70 instead of ACR20 or ACR50 instead of ACR20). Thus, these biomarkers are expected to serve as a diagnostic for patients most likely to benefit from therapies with anti-CD22 antibody and BAFF and APRIL antagonists such as anti-BR3 antibody, BR3-Fc, or atacicept.

In patients selected on the basis of the above biomarkers, anti-CD22 antibody and BAFF and APRIL antagonists such as anti-BR3 antibody, BR3-Fc, or atacicept are expected to exhibit superior efficacy compared to patients negative for the above biomarkers, for treatment of RA and for induction and maintenance of joint damage remission in such patients with the claimed markers. Such B-cell antagonists are expected to offer substantial advantages over standard therapy by virtue of their expected superior side-effect profiles, e.g., much less toxic than steroids, and better at restoring tolerance.

It is expected that the patients positively diagnosed and in the treatment arm will tolerate infusions of anti-CD22 antibody and BAFF/APRIL antagonists well and that their B cells will be depleted swiftly.

It is also expected that the patient diagnosed and treated with anti-CD22 antibodies and with BAFF and APRIL antagonists such as anti-BR3 antibody, BR3-Fc, or atacicept using a clinical protocol based on the parameters described in this specification and as known to those skilled in this art will show clinical improvement in the signs or symptoms of RA as evaluated by any one or more of the primary or secondary efficacy endpoints known for treating this disease. Moreover, the patient who is resistant or refractory to an immunosuppressive agent or another biological agent and who is treated, using a clinical protocol based on various parameters as described in this specification and as known to those skilled in the art, with the anti-CD22 antibody or BAFF or APRIL antagonist alone or in combination with a second medicament appropriate for the disease is expected to show greater improvement in any of the signs or symptoms of the RA, compared to the patient who continues on with the medicament to which he or she is resistant or refractory, or compared to the patient who is treated with only the second medicament appropriate for the disease and not with the anti-CD22 antibody or the BAFF or APRIL antagonist.

What is claimed is:

1. A method of treating rheumatoid arthritis in a patient comprising administering an effective amount of a B-cell antagonist to the patient to treat the rheumatoid arthritis, provided that a PTPN22 R620W single-nucleotide polymorphism (SNP) or shared epitope or both SNP and shared epitope are present in a genetic sample from the patient.

2. The method of claim 1 wherein the SNP is present, but not the shared epitope.

3. The method of claim 1 wherein the shared epitope is present, but not the SNP.

4. The method of claim 1 wherein both the SNP and shared epitope are present.

5. The method of claim 1 wherein samples from the patient do not reveal any biomarker indicating responsiveness of the patient to B-cell antagonist treatment other than the SNP or shared epitope or both.

6. The method of claim 1 wherein samples from the patient do reveal one or more biomarkers indicating responsiveness of the patient to B-cell antagonist treatment other than the SNP or shared epitope or both.

7. The method of claim 6 wherein a sample from the patient is seropositive for one or both of the additional biomarkers anti-CCP antibody and rheumatoid factor.

8. The method of claim 7 wherein the additional biomarker is anti-CCP antibody.

9. The method of claim 8 wherein the antibody is of the IgG isotype.

10. The method of claim 8 wherein the antibody is of the IgM isotype.

11. The method of claim 7 wherein the additional biomarker is a rheumatoid factor.

12. The method of claim 11 wherein the rheumatoid factor has an IgA, IgG, or IgM isotype.

13. The method of claim 7 wherein the additional biomarkers are both anti-CCP antibody and rheumatoid factor.

14. The method of claim 7 wherein a patient sample shows the presence of the shared epitope but not the SNP, and a patient sample is seropositive for rheumatoid factor, but not for anti-CCP antibody.

15. The method of claim 7 wherein a patient sample shows the presence of the SNP but not the shared epitope, and a patient sample is seropositive for anti-CCP antibody, but not for rheumatoid factor.

16. The method of claim 1 wherein the antagonist is an antibody or immunoadhesin.

17. The method of claim 1 wherein the antagonist is to CD20, CD22, BAFF, or APRIL.

18. The method of claim 1 wherein the antagonist is an antibody or TACI-Ig.

19. The method of claim 18 wherein the antibody is a chimeric, humanized, or human antibody.

20. The method of claim 18 wherein the antagonist is anti-CD20 antibody or anti-CD22 antibody.

21. The method of claim 20 wherein the antagonist is anti-CD20 antibody.

22. The method of claim 21 wherein the anti-CD20 antibody is rituximab.

23. The method of claim 21 wherein the anti-CD20 antibody is a 2H7 antibody.

24. The method of claim 23 wherein the 2H7 antibody comprises the L-chain variable region sequence of SEQ ID NO:1 and the H-chain variable region sequence of SEQ ID NO:2.

25. The method of claim 23 wherein the 2H7 antibody comprises the L-chain variable region sequence of SEQ ID NO:3 and the H-chain variable region sequence of SEQ ID NO:4.

26. The method of claim 23 wherein the 2H7 antibody comprises the L-chain variable region sequence of SEQ ID NO:3 and the H-chain variable region sequence of SEQ ID NO:5.

27. The method of claim 23 wherein the 2H7 antibody comprises the full-length L chain of SEQ ID NO:6 and the full-length H chain of SEQ ID NO:7.

28. The method of claim 23 wherein the 2H7 antibody comprises the full-length L chain of SEQ ID NO:6 and the full-length H chain of SEQ ID NO:8.

29. The method of claim 23 wherein the 2H7 antibody comprises the full-length L chain of SEQ ID NO:9 and the full-length H chain of SEQ ID NO:10.

30. The method of claim 23 wherein the 2H7 antibody comprises the full-length L chain of SEQ ID NO:9 and the full-length H chain of SEQ ID NO:11.

31. The method of claim 23 wherein the 2H7 antibody comprises the full-length L chain of SEQ ID NO:9 and the full-length H chain of SEQ ID NO:12.

32. The method of claim 23 wherein the 2H7 antibody comprises the full-length L chain of SEQ ID NO:9 and the full-length H chain of SEQ ID NO:13.

33. The method of claim 23 wherein the 2H7 antibody comprises the full-length L chain of SEQ ID NO:9 and the full-length H chain of SEQ ID NO:14.

34. The method of claim 23 wherein the 2H7 antibody comprises the full-length L chain of SEQ ID NO:6 and the full-length H chain of SEQ ID NO:15.

35. The method of claim 1 wherein the antagonist is not conjugated with a cytotoxic agent.

36. The method of claim 1 wherein the antagonist is conjugated with a cytotoxic agent.

37. The method of claim 1 wherein the genetic sample is blood, synovial tissue, or synovial fluid.

38. The method of claim 37 wherein the sample is blood.

39. The method of claim 1 wherein the patient has never been previously administered a medicament for the rheumatoid arthritis.

40. The method of claim 1 wherein the patient has been previously administered at least one medicament for the rheumatoid arthritis.

41. The method of claim 40 wherein the patient was not responsive to at least one medicament that was previously administered.

42. The method of claim 41 wherein the previously administered medicament or medicaments are an immunosuppressive agent, cytokine antagonist, integrin antagonist, corticosteroid, analgesic, a disease-modifying anti-rheumatic drug (DMARD), or a non-steroidal anti-inflammatory drug (NSAID).

43. The method of claim 42 wherein the previously administered medicament or medicaments are an immunosuppressive agent, cytokine antagonist, integrin antagonist, corticosteroid, DMARD, or NSAID.

44. The method of claim 42 wherein the previously administered medicament is a TNF-α inhibitor or methotrexate.

45. The method of claim 42 wherein the previously administered medicament is a CD20 antagonist that is not rituximab or a 2H7 antibody.

46. The method of claim 42 wherein the previously administered medicament is rituximab or a 2H7 antibody.

47. The method of claim 1 wherein the B-cell antagonist is administered intravenously.

48. The method of claim 1 wherein the B-cell antagonist is administered subcutaneously.

49. The method of claim 1 wherein at least about three months after the administration, an imaging test is given that measures a reduction in bone or soft tissue joint damage as compared to baseline prior to the administration, and the amount of the B-cell antagonist administered is effective in achieving a reduction in the joint damage.

50. The method of claim 49 wherein the test measures a total modified Sharp score.

51. The method of claim 1 wherein the antagonist is administered in a dose of about 0.2 to 4 grams.

52. The method of claim 51 wherein the dose is about 0.2 to 3.5 grams.

53. The method of claim 52 wherein the dose is about 0.4 to 2.5 grams.

54. The method of claim 53 wherein the dose is about 0.5 to 1.5 grams.

55. The method of claim 1 wherein the antagonist is administered at a frequency of one to four doses within a period of about one month.

56. The method of claim 55 wherein the antagonist is an anti-CD20 antibody and the dose is about 200 mg to 1.2 grams.

57. The method of claim 56 wherein the dose is about 200 mg to 1.1 grams.

58. The method of claim 55 wherein the antagonist is administered in two to three doses.

59. The method of claim 55 wherein the antagonist is administered within a period of about 2 to 3 weeks.

60. The method of claim 1 wherein the B-cell antagonist is administered without any other medicament to treat the RA.

61. The method of claim 1 further comprising administering an effective amount of one or more second medicaments with the B-cell antagonist, wherein the B-cell antagonist is a first medicament.

62. The method of claim 61 wherein the second medicament is more than one medicament.

63. The method of claim 61 wherein the second medicament is an immunosuppressive agent, a disease-modifying anti-rheumatic drug (DMARD), a pain-control agent, an integrin antagonist, a non-steroidal anti-inflammatory drug (NSAID), a cytokine antagonist, a bisphosphonate, or a combination thereof.

64. The method of claim 63 wherein the second medicament is a DMARD.

65. The method of claim 64 wherein the DMARD is selected from the group consisting of auranofin, chloroquine, D-penicillamine, injectable gold, oral gold, hydroxychloroquine, sulfasalazine, myocrisin and methotrexate.

66. The method of claim 63 wherein the second medicament is a NSAID.

67. The method of claim 66 wherein the NSAID is selected from the group consisting of: fenbufen, naprosyn, diclofenac, etodolac, indomethacin, aspirin and ibuprofen.

68. The method of claim 63 wherein the immunosuppressive agent is selected from the group consisting of etanercept, infliximab, adalimumab, leflunomide, anakinra, azathioprine, and cyclophosphamide.

69. The method of claim 63 wherein the second medicament is selected from the group consisting of anti-alpha4, etanercept, infliximab, etanercept, adalimumab, kinaret, efalizumab, osteoprotegerin (OPG), anti-receptor activator of NFκB ligand (anti-RANKL), anti-receptor activator of NFκB-Fc (RANK-Fc), pamidronate, alendronate, actonel, zolendronate, clodronate, methotrexate, azulfidine, hydroxychloroquine, doxycycline, leflunomide, sulfasalazine (SSZ), prednisolone, interleukin-1 receptor antagonist, prednisone, and methylprednisolone.

70. The method of claim 63 wherein the second medicament is selected from the group consisting of infliximab, an infliximab/methotrexate (MTX) combination, MTX, etanercept, a corticosteroid, cyclosporin A, azathioprine, auranofin, hydroxychloroquine (HCQ), combination of prednisolone, MTX, and SSZ, combinations of MTX, SSZ, and HCQ, the combination of cyclophosphamide, azathioprine, and HCQ, and the combination of adalimumab with MTX.

71. The method of claim 70 wherein the corticosteroid is prednisone, prednisolone, methylprednisolone, hydrocortisone, or dexamethasone.

72. The method of claim 70 wherein the second medicament is MTX.

73. The method of claim 72 wherein the MTX is administered perorally or parenterally.

74. The method of claim 1 wherein the B-cell antagonist is an anti-CD20 antibody administered at a dose of about 1000 mg×2 on days 1 and 15 intravenously at the start of the treatment.

75. The method of claim 74 wherein the anti-CD20 antibody is administered as a single dose or as two infusions, with each dose at about 200 mg to 600 mg.

76. The method of claim 1 wherein the arthritis is early rheumatoid arthritis or incipient rheumatoid arthritis.

77. The method of claim 1 further comprising re-treating the patient by administering an effective amount of the B-cell antagonist to the patient, wherein the re-treatment is commenced at least about 24 weeks after the first administration of the antagonist.

78. The method of claim 77 wherein a further re-treatment is commenced with an effective amount of the B-cell antagonist.

79. The method of claim 78 wherein the further re-treatment is commenced at least about 24 weeks after the second administration of the antagonist.

80. The method of claim 77 wherein the amount of the B-cell antagonist administered upon each administration thereof is effective to achieve a continued or maintained reduction in joint damage.

81. A method of treating rheumatoid arthritis in a patient comprising first administering a B-cell antagonist to the patient to treat the rheumatoid arthritis, provided that a PTPN22 R620W single-nucleotide polymorphism (SNP) or shared epitope or both SNP and shared epitope are present in a genetic sample from the patient, and at least about 24 weeks after the first administration of the antagonist, re-treating the patient by administering an effective amount of the B-cell antagonist to the patient, wherein no clinical improvement is observed in the patient at the time of the testing after the first administration of the B-cell antagonist.

82. The method of claim 81 wherein the clinical improvement is determined by assessing the number of tender or swollen joints, conducting a global clinical assessment of the patient, assessing erythrocyte sedimentation rate, assessing the amount of C-reactive protein level, or using composite measures of disease activity.

83. The method of claim 81 wherein the amount of the B-cell antagonist administered upon re-treatment is effective to achieve a continued or maintained reduction in joint damage as compared to the effect of a prior administration of the B-cell antagonist.

84. A method of treating rheumatoid arthritis in a patient comprising administering to the patient an effective amount of a B-cell antagonist, wherein before the administration, expression of PTPN22 R620W single-nucleotide polymorphism (SNP), or shared epitope, or both SNP and shared epitope was detected in a genetic sample from the patient.

85. A method of treating rheumatoid arthritis in a patient comprising administering to the patient an effective amount of a B-cell antagonist, wherein before the administration a genetic sample from the patient was determined to exhibit expression of PTPN22 R620W single-nucleotide polymorphism (SNP), or shared epitope, or both SNP and shared epitope, whereby the expression indicates that the patient will respond to treatment with the antagonist.

86. A method of treating rheumatoid arthritis in a patient comprising administering to the patient an effective amount of a B-cell antagonist, wherein before the administration a genetic sample from the patient was determined to exhibit expression of PTPN22 R620W single-nucleotide polymorphism (SNP), or shared epitope, or both SNP and shared epitope, whereby the expression indicates that the patient is likely to respond favorably to treatment with the antagonist.

87. A method for advertising a B-cell antagonist or a pharmaceutically acceptable composition thereof comprising promoting, to a target audience, the use of the antagonist or pharmaceutical composition thereof for treating a patient or patient population with rheumatoid arthritis from whom a genetic sample has been obtained showing the presence of a PTPN22 R620W single-nucleotide polymorphism (SNP) or shared epitope, or both SNP and shared epitope.

88. An article of manufacture comprising, packaged together, a pharmaceutical composition comprising a B-cell antagonist and a pharmaceutically acceptable carrier and a label stating that the antagonist or pharmaceutical composition is indicated for treating patients with rheumatoid arthritis from whom a genetic sample has been obtained showing the presence of a PTPN22 R620W single-nucleotide polymorphism (SNP) or shared epitope, or both SNP and shared epitope.

89. The article of claim 88 further comprising a container comprising a second medicament, wherein the B-cell antagonist is a first medicament, further comprising instructions on the package insert for treating the patient with an effective amount of the second medicament.

90. The article of claim 89 wherein the second medicament is methotrexate.

91. A method for manufacturing a B-cell antagonist or a pharmaceutical composition thereof comprising combining in a package the antagonist or pharmaceutical composition and a label stating that the antagonist or pharmaceutical composition is indicated for treating patients with rheumatoid arthritis from whom a genetic sample has been obtained showing the presence of a PTPN22 R620W single-nucleotide polymorphism (SNP) or shared epitope, or both SNP and shared epitope.

92. A method of providing a treatment option for patients with rheumatoid arthritis comprising packaging a B-cell antagonist in a vial with a package insert containing instructions to treat patients with rheumatoid arthritis from whom a genetic sample has been obtained showing the presence of a PTPN22 R620W single-nucleotide polymorphism (SNP) or shared epitope, or both SNP and shared epitope.

93. A method for predicting whether a subject with rheumatoid arthritis will respond to a B-cell antagonist, the method comprising determining whether a genetic sample from the subject shows the presence of a PTPN22 R620W single-nucleotide polymorphism (SNP) or shared epitope, or both SNP and shared epitope, wherein said presence indicates that the subject will respond to the antagonist.

94. A method of specifying a B-cell antagonist for use in a rheumatoid arthritis patient subpopulation, the method comprising providing instruction to administer the B-cell antagonist to a patient subpopulation characterized by the presence of a PTPN22 R620W single-nucleotide polymorphism (SNP) or shared epitope, or both SNP and shared epitope.

95. A method for marketing a B-cell antagonist for use in a rheumatoid arthritis patient subpopulation, the method comprising informing a target audience about the use of the antagonist for treating the patient subpopulation characterized by the presence, in patients of such subpopulation, of a PTPN22 R620W single-nucleotide polymorphism (SNP) or shared epitope, or both SNP and shared epitope.

96. A method of assessing whether a sample from a patient with rheumatoid arthritis indicates responsiveness of the patient to treatment with a B-cell antagonist comprising:
a. detecting in the sample whether at least one biomarker that is PTPN22 R620W single-nucleotide polymorphism (SNP) or shared epitope is present;
b. implementing an algorithm to determine that the patient is responsive to said treatment; and
c. recording a result specific to the sample being tested.

97. The method of claim 96 wherein a computer or machine is used to record the result specific to the sample being tested.

98. A system for analyzing susceptibility or responsiveness of a patient with rheumatoid arthritis to treatment with a B-cell antagonist comprising:
a. reagents to detect in a sample from the patient the biomarker PTPN22 R620W single-nucleotide polymorphism (SNP) or shared epitope, or both biomarkers SNP and shared epitope;
b. hardware to perform detection of the biomarkers; and
c. computational means to perform an algorithm to determine if the patient is susceptible or responsive to said treatment.
